SEQUENCE LISTING (579 sequences)

SEQ ID NO: 1 - DNA, 23 bp, Artificial Sequence
  Description of Artificial Sequence Synthetic oligonucleotide
tggccctactgcatcagatccaa

SEQ ID NO: 2 - DNA, 22 bp, Artificial Sequence
  Description of Artificial Sequence Synthetic oligonucleotide
atcagatccaagggaacgagct

SEQ ID NO: 3 - DNA, 21 bp, Artificial Sequence
  Description of Artificial Sequence Synthetic oligonucleotide
gcttgctgctgtccaggtaca

SEQ ID NO: 4 - DNA, 18 bp, Artificial Sequence
  Description of Artificial Sequence Synthetic oligonucleotide
cgtggtgcccctctatgg

SEQ ID NO: 5 - DNA, 18 bp, Artificial Sequence
  Description of Artificial Sequence Synthetic oligonucleotide
cgtggtgcccctctatag

SEQ ID NO: 6 - DNA, 18 bp, Artificial Sequence
  Description of Artificial Sequence Synthetic oligonucleotide
cgtggtgcccctctatcg

SEQ ID NO: 7 - DNA, 18 bp, Artificial Sequence
  Description of Artificial Sequence Synthetic oligonucleotide
cgtggtgcccctctattg

SEQ ID NO: 8 - DNA, 18 bp, Artificial Sequence
  Description of Artificial Sequence Synthetic oligonucleotide
cgtggtgcccctctaagg

SEQ ID NO: 9 - DNA, 18 bp, Artificial Sequence
  Description of Artificial Sequence Synthetic oligonucleotide
cgtggtgcccctctacgg

SEQ ID NO: 10 - DNA, 18 bp, Artificial Sequence
  Description of Artificial Sequence Synthetic oligonucleotide
cgtggtgcccctctaggg

SEQ ID NO: 11 - DNA, 18 bp, Artificial Sequence
  Description of Artificial Sequence Synthetic oligonucleotide
cgtggtgcccctctctgg

SEQ ID NO: 12 - DNA, 18 bp, Artificial Sequence
  Description of Artificial Sequence Synthetic oligonucleotide
cgtggtgcccctctgtgg

SEQ ID NO: 13 - DNA, 18 bp, Artificial Sequence
  Description of Artificial Sequence Synthetic oligonucleotide
cgtggtgcccctctttgg

SEQ ID NO: 14 - DNA, 18 bp, Artificial Sequence
  Description of Artificial Sequence Synthetic oligonucleotide
gcatctccagcagcaggc

SEQ ID NO: 15 - DNA, 18 bp, Artificial Sequence
  Description of Artificial Sequence Synthetic oligonucleotide
gcatctccagcagcagac

SEQ ID NO: 16 - DNA, 18 bp, Artificial Sequence
  Description of Artificial Sequence Synthetic oligonucleotide
gcatctccagcagcagcc

SEQ ID NO: 17 - DNA, 18 bp, Artificial Sequence
  Description of Artificial Sequence Synthetic oligonucleotide
gcatctccagcagcagtc

SEQ ID NO: 18 - DNA, 18 bp, Artificial Sequence
  Description of Artificial Sequence Synthetic oligonucleotide
gcatctccagcagcaagc

SEQ ID NO: 19 - DNA, 18 bp, Artificial Sequence
  Description of Artificial Sequence Synthetic oligonucleotide
gcatctccagcagcacgc

SEQ ID NO: 20 - DNA, 18 bp, Artificial Sequence
  Description of Artificial Sequence Synthetic oligonucleotide
gcatctccagcagcatgc

SEQ ID NO: 21 - DNA, 18 bp, Artificial Sequence
  Description of Artificial Sequence Synthetic oligonucleotide
gcatctccagcagccggc

SEQ ID NO: 22 - DNA, 18 bp, Artificial Sequence
  Description of Artificial Sequence Synthetic oligonucleotide
gcatctccagcagcgggc

SEQ ID NO: 23 - DNA, 18 bp, Artificial Sequence
  Description of Artificial Sequence Synthetic oligonucleotide
gcatctccagcagctggc

SEQ ID NO: 24 - DNA, 18 bp, Artificial Sequence
  Description of Artificial Sequence Synthetic oligonucleotide
gcatctccagcagcgggc

SEQ ID NO: 25 - DNA, 20 bp, Artificial Sequence
  Description of Artificial Sequence Synthetic oligonucleotide
cagcatctccagcagctggc

SEQ ID NO: 26 - DNA, 20 bp, Artificial Sequence
  Description of Artificial Sequence Synthetic oligonucleotide
cagcatctccagcagcaggc

SEQ ID NO: 27 - DNA, 20 bp, Artificial Sequence
  Description of Artificial Sequence Synthetic oligonucleotide
cagcatctccagcagcgggc

SEQ ID NO: 28 - DNA, 16 bp, Artificial Sequence
  Description of Artificial Sequence Synthetic oligonucleotide
tgagtcagcagggttt

SEQ ID NO: 29 - DNA, 21 bp, Artificial Sequence
  Description of Artificial Sequence Synthetic oligonucleotide
taggagcaaacagtagcttcc

SEQ ID NO: 30 - DNA, 24 bp, Artificial Sequence
  Description of Artificial Sequence Synthetic oligonucleotide
agttaggagcaaacagtagcttcc

SEQ ID NO: 31 - DNA, 30 bp, Artificial Sequence
  Description of Artificial Sequence Synthetic oligonucleotide
tgtgcctggctagagatcctgatgattggt

SEQ ID NO: 32 - DNA, 23 bp, Artificial Sequence
  Description of Artificial Sequence Synthetic oligonucleotide
ccatgatcaggtccaccttctac

SEQ ID NO: 33 - DNA, 23 bp, Artificial Sequence
  Description of Artificial Sequence Synthetic oligonucleotide
ccatgatcaggtccaccttctcc

SEQ ID NO: 34 - DNA, 23 bp, Artificial Sequence
  Description of Artificial Sequence Synthetic oligonucleotide
ccatgatcaggtccaccttctgc

SEQ ID NO: 35 - DNA, 23 bp, Artificial Sequence
  Description of Artificial Sequence Synthetic oligonucleotide
ccatgatcaggtccaccttcttc

SEQ ID NO: 36 - DNA, 23 bp, Artificial Sequence
  Description of Artificial Sequence Synthetic oligonucleotide
ccatgatcaggtccaccttcaac

SEQ ID NO: 37 - DNA, 23 bp, Artificial Sequence
  Description of Artificial Sequence Synthetic oligonucleotide
ccatgatcaggtccaccttccac

SEQ ID NO: 38 - DNA, 23 bp, Artificial Sequence
  Description of Artificial Sequence Synthetic oligonucleotide
ccatgatcaggtccaccttcgac

SEQ ID NO: 39 - DNA, 23 bp, Artificial Sequence
  Description of Artificial Sequence Synthetic oligonucleotide
ccatgatcaggtccaccttatac

SEQ ID NO: 40 - DNA, 23 bp, Artificial Sequence
  Description of Artificial Sequence Synthetic oligonucleotide
ccatgatcaggtccaccttgtac

SEQ ID NO: 41 - DNA, 23 bp, Artificial Sequence
  Description of Artificial Sequence Synthetic oligonucleotide
ccatgatcaggtccaccttttac

SEQ ID NO: 42 - DNA, 25 bp, Artificial Sequence
  Description of Artificial Sequence Synthetic oligonucleotide
ctccatgatcaggtccaccttgtac

SEQ ID NO: 43 - DNA, 30 bp, Artificial Sequence
  Description of Artificial Sequence Synthetic oligonucleotide
tggacctgatcatggagggtcaaatccaca

SEQ ID NO: 44 - DNA, 22 bp, Artificial Sequence
  Description of Artificial Sequence Synthetic oligonucleotide
ggatctctagccaggcacattg

SEQ ID NO: 45 - DNA, 22 bp, Artificial Sequence
  Description of Artificial Sequence Synthetic oligonucleotide
ggatctctagccaggcacatag

SEQ ID NO: 46 - DNA, 22 bp, Artificial Sequence
  Description of Artificial Sequence Synthetic oligonucleotide
ggatctctagccaggcacatcg

SEQ ID NO: 47 - DNA, 22 bp, Artificial Sequence
  Description of Artificial Sequence Synthetic oligonucleotide
ggatctctagccaggcacatgg

SEQ ID NO: 48 - DNA, 22 bp, Artificial Sequence
  Description of Artificial Sequence Synthetic oligonucleotide
ggatctctagccaggcacaatg

SEQ ID NO: 49 - DNA, 22 bp, Artificial Sequence
  Description of Artificial Sequence Synthetic oligonucleotide
ggatctctagccaggcacactg

SEQ ID NO: 50 - DNA, 22 bp, Artificial Sequence
  Description of Artificial Sequence Synthetic oligonucleotide
ggatctctagccaggcacagtg

SEQ ID NO: 51 - DNA, 22 bp, Artificial Sequence
  Description of Artificial Sequence Synthetic oligonucleotide
ggatctctagccaggcaccttg

SEQ ID NO: 52 - DNA, 22 bp, Artificial Sequence
  Description of Artificial Sequence Synthetic oligonucleotide
ggatctctagccaggcacgttg

SEQ ID NO: 53 - DNA, 22 bp, Artificial Sequence
  Description of Artificial Sequence Synthetic oligonucleotide
ggatctctagccaggcactttg

SEQ ID NO: 54 - DNA, 25 bp, Artificial Sequence
  Description of Artificial Sequence Synthetic oligonucleotide
ccctgaaccgtccgcagctcaagat

SEQ ID NO: 55 - DNA, 20 bp, Artificial Sequence
  Description of Artificial Sequence Synthetic oligonucleotide
ccctactgcatcagatccaa

SEQ ID NO: 56 - DNA, 20 bp, Artificial Sequence
  Description of Artificial Sequence Synthetic oligonucleotide
atcagatccaagggaacgag

SEQ ID NO: 57 - DNA, 19 bp, Artificial Sequence
  Description of Artificial Sequence Synthetic oligonucleotide
ttgctgctgtccaggtaca

SEQ ID NO: 58 - DNA, 24 bp, Artificial Sequence
  Description of Artificial Sequence Synthetic oligonucleotide
tgctatgttttcataggaaccagg

SEQ ID NO: 59 - DNA, 22 bp, Artificial Sequence
  Description of Artificial Sequence Synthetic oligonucleotide
gatttgaggcacacaaactcct

SEQ ID NO: 60 - DNA, 30 bp, Artificial Sequence
  Description of Artificial Sequence Synthetic oligonucleotide
tcatctcggttccgcatgatgaatctgcag

SEQ ID NO: 61 - DNA, 21 bp, Artificial Sequence
  Description of Artificial Sequence Synthetic oligonucleotide
tggagatcttcgacatgctgg

SEQ ID NO: 62 - DNA, 21 bp, Artificial Sequence
  Description of Artificial Sequence Synthetic oligonucleotide
tggagatcttcgacatgctag

SEQ ID NO: 63 - DNA, 21 bp, Artificial Sequence
  Description of Artificial Sequence Synthetic oligonucleotide
tggagatcttcgacatgctcg

SEQ ID NO: 64 - DNA, 21 bp, Artificial Sequence
  Description of Artificial Sequence Synthetic oligonucleotide
tggagatcttcgacatgcttg

SEQ ID NO: 65 - DNA, 21 bp, Artificial Sequence
  Description of Artificial Sequence Synthetic oligonucleotide
tggagatcttcgacatgcagg

SEQ ID NO: 66 - DNA, 21 bp, Artificial Sequence
  Description of Artificial Sequence Synthetic oligonucleotide
tggagatcttcgacatgccgg

SEQ ID NO: 67 - DNA, 21 bp, Artificial Sequence
  Description of Artificial Sequence Synthetic oligonucleotide
tggagatcttcgacatgcggg

SEQ ID NO: 68 - DNA, 21 bp, Artificial Sequence
  Description of Artificial Sequence Synthetic oligonucleotide
tggagatcttcgacatgatgg

SEQ ID NO: 69 - DNA, 21 bp, Artificial Sequence
  Description of Artificial Sequence Synthetic oligonucleotide
tggagatcttcgacatggtgg

SEQ ID NO: 70 - DNA, 21 bp, Artificial Sequence
  Description of Artificial Sequence Synthetic oligonucleotide
tggagatcttcgacatgttgg

SEQ ID NO: 71 - DNA, 30 bp, Artificial Sequence
  Description of Artificial Sequence Synthetic oligonucleotide
tcgaagatctccaccatgccctctacacat

SEQ ID NO: 72 - DNA, 22 bp, Artificial Sequence
  Description of Artificial Sequence Synthetic oligonucleotide
ggaaccgagatgatgtagccac

SEQ ID NO: 73 - DNA, 22 bp, Artificial Sequence
  Description of Artificial Sequence Synthetic oligonucleotide
ggaaccgagatgatgtagcccc

SEQ ID NO: 74 - DNA, 22 bp, Artificial Sequence
  Description of Artificial Sequence Synthetic oligonucleotide
ggaaccgagatgatgtagccgc

SEQ ID NO: 75 - DNA, 22 bp, Artificial Sequence
  Description of Artificial Sequence Synthetic oligonucleotide
ggaaccgagatgatgtagcctc

SEQ ID NO: 76 - DNA, 22 bp, Artificial Sequence
  Description of Artificial Sequence Synthetic oligonucleotide
ggaaccgagatgatgtagcaac

SEQ ID NO: 77 - DNA, 22 bp, Artificial Sequence
  Description of Artificial Sequence Synthetic oligonucleotide
ggaaccgagatgatgtagcgac

SEQ ID NO: 78 - DNA, 22 bp, Artificial Sequence
  Description of Artificial Sequence Synthetic oligonucleotide
ggaaccgagatgatgtagctac

SEQ ID NO: 79 - DNA, 22 bp, Artificial Sequence
  Description of Artificial Sequence Synthetic oligonucleotide
ggaaccgagatgatgtagacac

SEQ ID NO: 80 - DNA, 22 bp, Artificial Sequence
  Description of Artificial Sequence Synthetic oligonucleotide
ggaaccgagatgatgtaggcac

SEQ ID NO: 81 - DNA, 22 bp, Artificial Sequence
  Description of Artificial Sequence Synthetic oligonucleotide
ggaaccgagatgatgtagtcac

SEQ ID NO: 82 - DNA, 24 bp, Artificial Sequence
  Description of Artificial Sequence Synthetic oligonucleotide
ctttctgtgtcttcccacctacag

SEQ ID NO: 83 - DNA, 18 bp, Artificial Sequence
  Description of Artificial Sequence Synthetic oligonucleotide
aagtggctttggtccgtc

SEQ ID NO: 84 - DNA, 15 bp, Artificial Sequence
  Description of Artificial Sequence Synthetic oligonucleotide
ccagcagcagggggg

SEQ ID NO: 85 - DNA, 14 bp, Artificial Sequence
  Description of Artificial Sequence Synthetic oligonucleotide
cagcagcagggggg

SEQ ID NO: 86 - DNA, 14 bp, Artificial Sequence
  Description of Artificial Sequence Synthetic oligonucleotide
ccagcagcaggggg

SEQ ID NO: 87 - DNA, 15 bp, Artificial Sequence
  Description of Artificial Sequence Synthetic oligonucleotide
tccagcagcaggggg

SEQ ID NO: 88 - DNA, 16 bp, Artificial Sequence
  Description of Artificial Sequence Synthetic oligonucleotide
ctccagcagcaggggg

SEQ ID NO: 89 - DNA, 15 bp, Artificial Sequence
  Description of Artificial Sequence Synthetic oligonucleotide
ctccagcagcagggg

SEQ ID NO: 90 - DNA, 16 bp, Artificial Sequence
  Description of Artificial Sequence Synthetic oligonucleotide
tctccagcagcagggg

SEQ ID NO: 91 - DNA, 17 bp, Artificial Sequence
  Description of Artificial Sequence Synthetic oligonucleotide
atctccagcagcagggg

SEQ ID NO: 92 - DNA, 17 bp, Artificial Sequence
  Description of Artificial Sequence Synthetic oligonucleotide
catctccagcagcaggg

SEQ ID NO: 93 - DNA, 18 bp, Artificial Sequence
  Description of Artificial Sequence Synthetic oligonucleotide
gcatctccagcagcaggg

SEQ ID NO: 94 - DNA, 23 bp, Artificial Sequence
  Description of Artificial Sequence Synthetic oligonucleotide
tacatgcgcccactagccgtgga

SEQ ID NO: 95 - DNA, 30 bp, Artificial Sequence
  Description of Artificial Sequence Synthetic oligonucleotide
tgcacttcatgctgtacagatgctccatgc

SEQ ID NO: 96 - DNA, 19 bp, Artificial Sequence
  Description of Artificial Sequence Synthetic oligonucleotide
gcaagaacgtggtgcccca

SEQ ID NO: 97 - DNA, 19 bp, Artificial Sequence
  Description of Artificial Sequence Synthetic oligonucleotide
gcaagaacgtggtgcccaa

SEQ ID NO: 98 - DNA, 19 bp, Artificial Sequence
  Description of Artificial Sequence Synthetic oligonucleotide
gcaagaacgtggtgcccga

SEQ ID NO: 99 - DNA, 19 bp, Artificial Sequence
  Description of Artificial Sequence Synthetic oligonucleotide
gcaagaacgtggtgcccta

SEQ ID NO: 100 - DNA, 19 bp, Artificial Sequence
  Description of Artificial Sequence Synthetic oligonucleotide
gcaagaacgtggtgccaca

SEQ ID NO: 101 - DNA, 19 bp, Artificial Sequence
  Description of Artificial Sequence Synthetic oligonucleotide
gcaagaacgtggtgccgca

SEQ ID NO: 102 - DNA, 19 bp, Artificial Sequence
  Description of Artificial Sequence Synthetic oligonucleotide
gcaagaacgtggtgcctca

SEQ ID NO: 103 - DNA, 19 bp, Artificial Sequence
  Description of Artificial Sequence Synthetic oligonucleotide
gcaagaacgtggtgcacca

SEQ ID NO: 104 - DNA, 19 bp, Artificial Sequence
  Description of Artificial Sequence Synthetic oligonucleotide
gcaagaacgtggtgcgcca

SEQ ID NO: 105 - DNA, 19 bp, Artificial Sequence
  Description of Artificial Sequence Synthetic oligonucleotide
gcaagaacgtggtgctcca

SEQ ID NO: 106 - DNA, 20 bp, Artificial Sequence
  Description of Artificial Sequence Synthetic oligonucleotide
ctccagcagcaggtcatagt

SEQ ID NO: 107 - DNA, 20 bp, Artificial Sequence
  Description of Artificial Sequence Synthetic oligonucleotide
ctccagcagcaggtcataat

SEQ ID NO: 108 - DNA, 20 bp, Artificial Sequence
  Description of Artificial Sequence Synthetic oligonucleotide
ctccagcagcaggtcatact

SEQ ID NO: 109 - DNA, 20 bp, Artificial Sequence
  Description of Artificial Sequence Synthetic oligonucleotide
ctccagcagcaggtcatatt

SEQ ID NO: 110 - DNA, 20 bp, Artificial Sequence
  Description of Artificial Sequence Synthetic oligonucleotide
ctccagcagcaggtcatcgt

SEQ ID NO: 111 - DNA, 20 bp, Artificial Sequence
  Description of Artificial Sequence Synthetic oligonucleotide
ctccagcagcaggtcatggt

SEQ ID NO: 112 - DNA, 20 bp, Artificial Sequence
  Description of Artificial Sequence Synthetic oligonucleotide
ctccagcagcaggtcattgt

SEQ ID NO: 113 - DNA, 20 bp, Artificial Sequence
  Description of Artificial Sequence Synthetic oligonucleotide
ctccagcagcaggtcaaagt

SEQ ID NO: 114 - DNA, 20 bp, Artificial Sequence
  Description of Artificial Sequence Synthetic oligonucleotide
ctccagcagcaggtcacagt

SEQ ID NO: 115 - DNA, 20 bp, Artificial Sequence
  Description of Artificial Sequence Synthetic oligonucleotide
ctccagcagcaggtcagagt

SEQ ID NO: 116 - DNA, 23 bp, Artificial Sequence
  Description of Artificial Sequence Synthetic oligonucleotide
catctccagcagcaggtcataat

SEQ ID NO: 117 - DNA, 23 bp, Artificial Sequence
  Description of Artificial Sequence Synthetic oligonucleotide
catctccagcagcaggtcatatt

SEQ ID NO: 118 - DNA, 23 bp, Artificial Sequence
  Description of Artificial Sequence Synthetic oligonucleotide
catctccagcagcaggtcgtagt

SEQ ID NO: 119 - DNA, 23 bp, Artificial Sequence
  Description of Artificial Sequence Synthetic oligonucleotide
catctccagcagcaggtcttagt

SEQ ID NO: 120 - DNA, 23 bp, Artificial Sequence
  Description of Artificial Sequence Synthetic oligonucleotide
catctccagcagcaggtcctagt

SEQ ID NO: 121 - DNA, 16 bp, Artificial Sequence
  Description of Artificial Sequence Synthetic oligonucleotide
agaacgtggtgccccc

SEQ ID NO: 122 - DNA, 16 bp, Artificial Sequence
  Description of Artificial Sequence Synthetic oligonucleotide
agaacgtggtgcccac

SEQ ID NO: 123 - DNA, 16 bp, Artificial Sequence
  Description of Artificial Sequence Synthetic oligonucleotide
agaacgtggtgcccgc

SEQ ID NO: 124 - DNA, 16 bp, Artificial Sequence
  Description of Artificial Sequence Synthetic oligonucleotide
agaacgtggtgccctc

SEQ ID NO: 125 - DNA, 16 bp, Artificial Sequence
  Description of Artificial Sequence Synthetic oligonucleotide
agaacgtggtgccacc

SEQ ID NO: 126 - DNA, 16 bp, Artificial Sequence
  Description of Artificial Sequence Synthetic oligonucleotide
agaacgtggtgccgcc

SEQ ID NO: 127 - DNA, 16 bp, Artificial Sequence
  Description of Artificial Sequence Synthetic oligonucleotide
agaacgtggtgcctcc

SEQ ID NO: 128 - DNA, 16 bp, Artificial Sequence
  Description of Artificial Sequence Synthetic oligonucleotide
agaacgtggtgcaccc

SEQ ID NO: 129 - DNA, 16 bp, Artificial Sequence
  Description of Artificial Sequence Synthetic oligonucleotide
agaacgtggtgcgccc

SEQ ID NO: 130 - DNA, 16 bp, Artificial Sequence
  Description of Artificial Sequence Synthetic oligonucleotide
agaacgtggtgctccc

SEQ ID NO: 131 - DNA, 20 bp, Artificial Sequence
  Description of Artificial Sequence Synthetic oligonucleotide
ctccagcagcaggtcatagg

SEQ ID NO: 132 - DNA, 20 bp, Artificial Sequence
  Description of Artificial Sequence Synthetic oligonucleotide
ctccagcagcaggtcataag

SEQ ID NO: 133 - DNA, 20 bp, Artificial Sequence
  Description of Artificial Sequence Synthetic oligonucleotide
ctccagcagcaggtcatacg

SEQ ID NO: 134 - DNA, 20 bp, Artificial Sequence
  Description of Artificial Sequence Synthetic oligonucleotide
ctccagcagcaggtcatatg

SEQ ID NO: 135 - DNA, 20 bp, Artificial Sequence
  Description of Artificial Sequence Synthetic oligonucleotide
ctccagcagcaggtcatcgg

SEQ ID NO: 136 - DNA, 20 bp, Artificial Sequence
  Description of Artificial Sequence Synthetic oligonucleotide
ctccagcagcaggtcatggg

SEQ ID NO: 137 - DNA, 20 bp, Artificial Sequence
  Description of Artificial Sequence Synthetic oligonucleotide
ctccagcagcaggtcattgg

SEQ ID NO: 138 - DNA, 20 bp, Artificial Sequence
  Description of Artificial Sequence Synthetic oligonucleotide
ctccagcagcaggtcaaagg

SEQ ID NO: 139 - DNA, 20 bp, Artificial Sequence
  Description of Artificial Sequence Synthetic oligonucleotide
ctccagcagcaggtcacagg

SEQ ID NO: 140 - DNA, 20 bp, Artificial Sequence
  Description of Artificial Sequence Synthetic oligonucleotide
ctccagcagcaggtcagagg

SEQ ID NO: 141 - DNA, 17 bp, Artificial Sequence
  Description of Artificial Sequence Synthetic oligonucleotide
agaacgtggtgccccag

SEQ ID NO: 142 - DNA, 17 bp, Artificial Sequence
  Description of Artificial Sequence Synthetic oligonucleotide
agaacgtggtgcccccg

SEQ ID NO: 143 - DNA, 17 bp, Artificial Sequence
  Description of Artificial Sequence Synthetic oligonucleotide
agaacgtggtgccccgg

SEQ ID NO: 144 - DNA, 17 bp, Artificial Sequence
  Description of Artificial Sequence Synthetic oligonucleotide
agaacgtggtgcccctg

SEQ ID NO: 145 - DNA, 17 bp, Artificial Sequence
  Description of Artificial Sequence Synthetic oligonucleotide
agaacgtggtgcccaag

SEQ ID NO: 146 - DNA, 17 bp, Artificial Sequence
  Description of Artificial Sequence Synthetic oligonucleotide
agaacgtggtgcccgag

SEQ ID NO: 147 - DNA, 17 bp, Artificial Sequence
  Description of Artificial Sequence Synthetic oligonucleotide
agaacgtggtgccctag

SEQ ID NO: 148 - DNA, 17 bp, Artificial Sequence
  Description of Artificial Sequence Synthetic oligonucleotide
agaacgtggtgccacag

SEQ ID NO: 149 - DNA, 17 bp, Artificial Sequence
  Description of Artificial Sequence Synthetic oligonucleotide
agaacgtggtgccgcag

SEQ ID NO: 150 - DNA, 17 bp, Artificial Sequence
  Description of Artificial Sequence Synthetic oligonucleotide
agaacgtggtgcctcag

SEQ ID NO: 151 - DNA, 20 bp, Artificial Sequence
  Description of Artificial Sequence Synthetic oligonucleotide
ctccagcagcaggtcatact

SEQ ID NO: 152 - DNA, 20 bp, Artificial Sequence
  Description of Artificial Sequence Synthetic oligonucleotide
ctccagcagcaggtcataat

SEQ ID NO: 153 - DNA, 20 bp, Artificial Sequence
  Description of Artificial Sequence Synthetic oligonucleotide
ctccagcagcaggtcatagt

SEQ ID NO: 154 - DNA, 20 bp, Artificial Sequence
  Description of Artificial Sequence Synthetic oligonucleotide
ctccagcagcaggtcatatt

SEQ ID NO: 155 - DNA, 20 bp, Artificial Sequence
  Description of Artificial Sequence Synthetic oligonucleotide
ctccagcagcaggtcatcct

SEQ ID NO: 156 - DNA, 20 bp, Artificial Sequence
  Description of Artificial Sequence Synthetic oligonucleotide
ctccagcagcaggtcatgct

SEQ ID NO: 157 - DNA, 20 bp, Artificial Sequence
  Description of Artificial Sequence Synthetic oligonucleotide
ctccagcagcaggtcattct

SEQ ID NO: 158 - DNA, 20 bp, Artificial Sequence
  Description of Artificial Sequence Synthetic oligonucleotide
ctccagcagcaggtcaaact

SEQ ID NO: 159 - DNA, 20 bp, Artificial Sequence
  Description of Artificial Sequence Synthetic oligonucleotide
ctccagcagcaggtcacact

SEQ ID NO: 160 - DNA, 20 bp, Artificial Sequence
  Description of Artificial Sequence Synthetic oligonucleotide
ctccagcagcaggtcagact

SEQ ID NO: 161 - DNA, 16 bp, Artificial Sequence
  Description of Artificial Sequence Synthetic oligonucleotide
agaacgtggtgccccg

SEQ ID NO: 162 - DNA, 16 bp, Artificial Sequence
  Description of Artificial Sequence Synthetic oligonucleotide
agaacgtggtgcccag

SEQ ID NO: 163 - DNA, 16 bp, Artificial Sequence
  Description of Artificial Sequence Synthetic oligonucleotide
agaacgtggtgcccgg

SEQ ID NO: 164 - DNA, 16 bp, Artificial Sequence
  Description of Artificial Sequence Synthetic oligonucleotide
agaacgtggtgccctg

SEQ ID NO: 165 - DNA, 16 bp, Artificial Sequence
  Description of Artificial Sequence Synthetic oligonucleotide
agaacgtggtgccacg

SEQ ID NO: 166 - DNA, 16 bp, Artificial Sequence
  Description of Artificial Sequence Synthetic oligonucleotide
agaacgtggtgccgcg

SEQ ID NO: 167 - DNA, 16 bp, Artificial Sequence
  Description of Artificial Sequence Synthetic oligonucleotide
agaacgtggtgcctcg

SEQ ID NO: 168 - DNA, 16 bp, Artificial Sequence
  Description of Artificial Sequence Synthetic oligonucleotide
agaacgtggtgcaccg

SEQ ID NO: 169 - DNA, 16 bp, Artificial Sequence
  Description of Artificial Sequence Synthetic oligonucleotide
agaacgtggtgcgccg

SEQ ID NO: 170 - DNA, 16 bp, Artificial Sequence
  Description of Artificial Sequence Synthetic oligonucleotide
agaacgtggtgctccg

SEQ ID NO: 171 - DNA, 20 bp, Artificial Sequence
  Description of Artificial Sequence Synthetic oligonucleotide
ctccagcagcaggtcatagc

SEQ ID NO: 172 - DNA, 20 bp, Artificial Sequence
  Description of Artificial Sequence Synthetic oligonucleotide
ctccagcagcaggtcataac

SEQ ID NO: 173 - DNA, 20 bp, Artificial Sequence
  Description of Artificial Sequence Synthetic oligonucleotide
ctccagcagcaggtcatacc

SEQ ID NO: 174 - DNA, 20 bp, Artificial Sequence
  Description of Artificial Sequence Synthetic oligonucleotide
ctccagcagcaggtcatatc

SEQ ID NO: 175 - DNA, 20 bp, Artificial Sequence
  Description of Artificial Sequence Synthetic oligonucleotide
ctccagcagcaggtcatcgc

SEQ ID NO: 176 - DNA, 20 bp, Artificial Sequence
  Description of Artificial Sequence Synthetic oligonucleotide
ctccagcagcaggtcatggc

SEQ ID NO: 177 - DNA, 20 bp, Artificial Sequence
  Description of Artificial Sequence Synthetic oligonucleotide
ctccagcagcaggtcattgc

SEQ ID NO: 178 - DNA, 20 bp, Artificial Sequence
  Description of Artificial Sequence Synthetic oligonucleotide
ctccagcagcaggtcaaagc

SEQ ID NO: 179 - DNA, 20 bp, Artificial Sequence
  Description of Artificial Sequence Synthetic oligonucleotide
ctccagcagcaggtcacagc

SEQ ID NO: 180 - DNA, 20 bp, Artificial Sequence
  Description of Artificial Sequence Synthetic oligonucleotide
ctccagcagcaggtcagagc

SEQ ID NO: 181 - DNA, 20 bp, Artificial Sequence
  Description of Artificial Sequence Synthetic oligonucleotide
aagtgcaagaacgtggtgca

SEQ ID NO: 182 - DNA, 20 bp, Artificial Sequence
  Description of Artificial Sequence Synthetic oligonucleotide
aagtgcaagaacgtggtgaa

SEQ ID NO: 183 - DNA, 20 bp, Artificial Sequence
  Description of Artificial Sequence Synthetic oligonucleotide
aagtgcaagaacgtggtgga

SEQ ID NO: 184 - DNA, 20 bp, Artificial Sequence
  Description of Artificial Sequence Synthetic oligonucleotide
aagtgcaagaacgtggtgta

SEQ ID NO: 185 - DNA, 20 bp, Artificial Sequence
  Description of Artificial Sequence Synthetic oligonucleotide
aagtgcaagaacgtggtaca

SEQ ID NO: 186 - DNA, 20 bp, Artificial Sequence
  Description of Artificial Sequence Synthetic oligonucleotide
aagtgcaagaacgtggtcca

SEQ ID NO: 187 - DNA, 20 bp, Artificial Sequence
  Description of Artificial Sequence Synthetic oligonucleotide
aagtgcaagaacgtggttca

SEQ ID NO: 188 - DNA, 20 bp, Artificial Sequence
  Description of Artificial Sequence Synthetic oligonucleotide
aagtgcaagaacgtggagca

SEQ ID NO: 189 - DNA, 20 bp, Artificial Sequence
  Description of Artificial Sequence Synthetic oligonucleotide
aagtgcaagaacgtggcgca

SEQ ID NO: 190 - DNA, 20 bp, Artificial Sequence
  Description of Artificial Sequence Synthetic oligonucleotide
aagtgcaagaacgtggggca

SEQ ID NO: 191 - DNA, 19 bp, Artificial Sequence
  Description of Artificial Sequence Synthetic oligonucleotide
agcagcaggtcatagaggt

SEQ ID NO: 192 - DNA, 19 bp, Artificial Sequence
  Description of Artificial Sequence Synthetic oligonucleotide
agcagcaggtcatagagat

SEQ ID NO: 193 - DNA, 19 bp, Artificial Sequence
  Description of Artificial Sequence Synthetic oligonucleotide
agcagcaggtcatagagct

SEQ ID NO: 194 - DNA, 19 bp, Artificial Sequence
  Description of Artificial Sequence Synthetic oligonucleotide
agcagcaggtcatagagtt

SEQ ID NO: 195 - DNA, 19 bp, Artificial Sequence
  Description of Artificial Sequence Synthetic oligonucleotide
agcagcaggtcatagaagt

SEQ ID NO: 196 - DNA, 19 bp, Artificial Sequence
  Description of Artificial Sequence Synthetic oligonucleotide
agcagcaggtcatagacgt

SEQ ID NO: 197 - DNA, 19 bp, Artificial Sequence
  Description of Artificial Sequence Synthetic oligonucleotide
agcagcaggtcatagatgt

SEQ ID NO: 198 - DNA, 19 bp, Artificial Sequence
  Description of Artificial Sequence Synthetic oligonucleotide
agcagcaggtcatagcggt

SEQ ID NO: 199 - DNA, 19 bp, Artificial Sequence
  Description of Artificial Sequence Synthetic oligonucleotide
agcagcaggtcataggggt

SEQ ID NO: 200 - DNA, 19 bp, Artificial Sequence
  Description of Artificial Sequence Synthetic oligonucleotide
agcagcaggtcatagtggt

SEQ ID NO: 201 - DNA, 21 bp, Artificial Sequence
  Description of Artificial Sequence Synthetic oligonucleotide
ccagcagcaggtcatagaggt

SEQ ID NO: 202 - DNA, 20 bp, Artificial Sequence
  Description of Artificial Sequence Synthetic oligonucleotide
ccagcagcaggtcatgmggt

SEQ ID NO: 203 - DNA, 20 bp, Artificial Sequence
  Description of Artificial Sequence Synthetic oligonucleotide
ccagcagcaggtcatagggt

SEQ ID NO: 204 - DNA, 21 bp, Artificial Sequence
  Description of Artificial Sequence Synthetic oligonucleotide
ccagcagcaggtcatagtggt

SEQ ID NO: 205 - DNA, 19 bp, Artificial Sequence
  Description of Artificial Sequence Synthetic oligonucleotide
agcagcaggtcataaaggt

SEQ ID NO: 206 - DNA, 19 bp, Artificial Sequence
  Description of Artificial Sequence Synthetic oligonucleotide
agcagcaggtcatacaggt

SEQ ID NO: 207 - DNA, 19 bp, Artificial Sequence
  Description of Artificial Sequence Synthetic oligonucleotide
agcagcaggtcatataggt

SEQ ID NO: 208 - DNA, 19 bp, Artificial Sequence
  Description of Artificial Sequence Synthetic oligonucleotide
agcagcaggtcatagaggt

SEQ ID NO: 209 - DNA, 19 bp, Artificial Sequence
  Description of Artificial Sequence Synthetic oligonucleotide
agcagcaggtcatagaggt

SEQ ID NO: 210 - DNA, 19 bp, Artificial Sequence
  Description of Artificial Sequence Synthetic oligonucleotide
agcagcaggtcatagaggt

SEQ ID NO: 211 - DNA, 19 bp, Artificial Sequence
  Description of Artificial Sequence Synthetic oligonucleotide
agcagcaggtcatagaggt

SEQ ID NO: 212 - DNA, 21 bp, Artificial Sequence
  Description of Artificial Sequence Synthetic oligonucleotide
ccagcagcaggtcatagcggt

SEQ ID NO: 213 - DNA, 19 bp, Artificial Sequence
  Description of Artificial Sequence Synthetic oligonucleotide
agcagcaggtcataaacgt

SEQ ID NO: 214 - DNA, 19 bp, Artificial Sequence
  Description of Artificial Sequence Synthetic oligonucleotide
agcagcaggtcatacacgt

SEQ ID NO: 215 - DNA, 19 bp, Artificial Sequence
  Description of Artificial Sequence Synthetic oligonucleotide
agcagcaggtcatatacgt

SEQ ID NO: 216 - DNA, 19 bp, Artificial Sequence
  Description of Artificial Sequence Synthetic oligonucleotide
agcagcaggtcataaatgt

SEQ ID NO: 217 - DNA, 19 bp, Artificial Sequence
  Description of Artificial Sequence Synthetic oligonucleotide
agcagcaggtcatacatgt

SEQ ID NO: 218 - DNA, 19 bp, Artificial Sequence
  Description of Artificial Sequence Synthetic oligonucleotide
agcagcaggtcatatatgt

SEQ ID NO: 219 - DNA, 19 bp, Artificial Sequence
  Description of Artificial Sequence Synthetic oligonucleotide
agcagcaggtcataaaagt

SEQ ID NO: 220 - DNA, 19 bp, Artificial Sequence
  Description of Artificial Sequence Synthetic oligonucleotide
agcagcaggtcatacaagt

SEQ ID NO: 221 - DNA, 19 bp, Artificial Sequence
  Description of Artificial Sequence Synthetic oligonucleotide
agcagcaggtcatataagt

SEQ ID NO: 222 - DNA, 19 bp, Artificial Sequence
  Description of Artificial Sequence Synthetic oligonucleotide
agcagcaggtcatagaggt

SEQ ID NO: 223 - DNA, 21 bp, Artificial Sequence
  Description of Artificial Sequence Synthetic oligonucleotide
ccagcagcaggtcatagaggt

SEQ ID NO: 224 - DNA, 21 bp, Artificial Sequence
  Description of Artificial Sequence Synthetic oligonucleotide
ccagcagcaggtcatagtggt

SEQ ID NO: 225 - DNA, 19 bp, Artificial Sequence
  Description of Artificial Sequence Synthetic oligonucleotide
agcagcaggtcatagaggt

SEQ ID NO: 226 - DNA, 20 bp, Artificial Sequence
  Description of Artificial Sequence Synthetic oligonucleotide
cagcagcaggtcataaaggt

SEQ ID NO: 227 - DNA, 20 bp, Artificial Sequence
  Description of Artificial Sequence Synthetic oligonucleotide
cagcagcaggtcatacaggt

SEQ ID NO: 228 - DNA, 20 bp, Artificial Sequence
  Description of Artificial Sequence Synthetic oligonucleotide
cagcagcaggtcatataggt

SEQ ID NO: 229 - DNA, 21 bp, Artificial Sequence
  Description of Artificial Sequence Synthetic oligonucleotide
ccagcagcaggtcataaaggt

SEQ ID NO: 230 - DNA, 21 bp, Artificial Sequence
  Description of Artificial Sequence Synthetic oligonucleotide
ccagcagcaggtcatacaggt

SEQ ID NO: 231 - DNA, 21 bp, Artificial Sequence
  Description of Artificial Sequence Synthetic oligonucleotide
ccagcagcaggtcatataggt

SEQ ID NO: 232 - DNA, 20 bp, Artificial Sequence
  Description of Artificial Sequence Synthetic oligonucleotide
agatggtcagtgccttgttg

SEQ ID NO: 233 - DNA, 20 bp, Artificial Sequence
  Description of Artificial Sequence Synthetic oligonucleotide
cagatggtcagtgccttgtt

SEQ ID NO: 234 - DNA, 22 bp, Artificial Sequence
  Description of Artificial Sequence Synthetic oligonucleotide
attcttacctggcaccctcttc

SEQ ID NO: 235 - DNA, 20 bp, Artificial Sequence
  Description of Artificial Sequence Synthetic oligonucleotide
ctcttcgcccagttgatcat

SEQ ID NO: 236 - DNA, 30 bp, Artificial Sequence
  Description of Artificial Sequence Synthetic oligonucleotide
agacagggagctggttcacatgatcaactg

SEQ ID NO: 237 - DNA, 30 bp, Artificial Sequence
  Description of Artificial Sequence Synthetic oligonucleotide
tgatgggcttactgaccaacctggcagaca

SEQ ID NO: 238 - DNA, 22 bp, Artificial Sequence
  Description of Artificial Sequence Synthetic oligonucleotide
ccagacccttcagtgaagcttt

SEQ ID NO: 239 - DNA, 22 bp, Artificial Sequence
  Description of Artificial Sequence Synthetic oligonucleotide
ccagacccttcagtgaagctat

SEQ ID NO: 240 - DNA, 22 bp, Artificial Sequence
  Description of Artificial Sequence Synthetic oligonucleotide
ccagacccttcagtgaagctct

SEQ ID NO: 241 - DNA, 22 bp, Artificial Sequence
  Description of Artificial Sequence Synthetic oligonucleotide
ccagacccttcagtgaagctgt

SEQ ID NO: 242 - DNA, 22 bp, Artificial Sequence
  Description of Artificial Sequence Synthetic oligonucleotide
ccagacccttcagtgaagcatt

SEQ ID NO: 243 - DNA, 22 bp, Artificial Sequence
  Description of Artificial Sequence Synthetic oligonucleotide
ccagacccttcagtgaagcctt

SEQ ID NO: 244 - DNA, 22 bp, Artificial Sequence
  Description of Artificial Sequence Synthetic oligonucleotide
ccagacccttcagtgaagcgtt

SEQ ID NO: 245 - DNA, 22 bp, Artificial Sequence
  Description of Artificial Sequence Synthetic oligonucleotide
ccagacccttcagtgaagattt

SEQ ID NO: 246 - DNA, 22 bp, Artificial Sequence
  Description of Artificial Sequence Synthetic oligonucleotide
ccagacccttcagtgaaggttt

SEQ ID NO: 247 - DNA, 22 bp, Artificial Sequence
  Description of Artificial Sequence Synthetic oligonucleotide
ccagacccttcagtgaagtttt

SEQ ID NO: 248 - DNA, 30 bp, Artificial Sequence
  Description of Artificial Sequence Synthetic oligonucleotide
actgaagggtctggtaggatcatactcgga

SEQ ID NO: 249 - DNA, 32 bp, Artificial Sequence
  Description of Artificial Sequence Synthetic oligonucleotide
ctgaagggtctggtaggatcatactcggaata

SEQ ID NO: 250 - DNA, 23 bp, Artificial Sequence
  Description of Artificial Sequence Synthetic oligonucleotide
gttggtcagtaagcccatcatca

SEQ ID NO: 251 - DNA, 23 bp, Artificial Sequence
  Description of Artificial Sequence Synthetic oligonucleotide
gttggtcagtaagcccatcataa

SEQ ID NO: 252 - DNA, 23 bp, Artificial Sequence
  Description of Artificial Sequence Synthetic oligonucleotide
gttggtcagtaagcccatcatga

SEQ ID NO: 253 - DNA, 23 bp, Artificial Sequence
  Description of Artificial Sequence Synthetic oligonucleotide
gttggtcagtaagcccatcatta

SEQ ID NO: 254 - DNA, 23 bp, Artificial Sequence
  Description of Artificial Sequence Synthetic oligonucleotide
gttggtcagtaagcccatcaaca

SEQ ID NO: 255 - DNA, 23 bp, Artificial Sequence
  Description of Artificial Sequence Synthetic oligonucleotide
gttggtcagtaagcccatcacca

SEQ ID NO: 256 - DNA, 23 bp, Artificial Sequence
  Description of Artificial Sequence Synthetic oligonucleotide
gttggtcagtaagcccatcagca

SEQ ID NO: 257 - DNA, 23 bp, Artificial Sequence
  Description of Artificial Sequence Synthetic oligonucleotide
gttggtcagtaagcccatcctca

SEQ ID NO: 258 - DNA, 23 bp, Artificial Sequence
  Description of Artificial Sequence Synthetic oligonucleotide
gttggtcagtaagcccatcgtca

SEQ ID NO: 259 - DNA, 23 bp, Artificial Sequence
  Description of Artificial Sequence Synthetic oligonucleotide
gttggtcagtaagcccatcttca

SEQ ID NO: 260 - DNA, 22 bp, Artificial Sequence
  Description of Artificial Sequence Synthetic oligonucleotide
ctagacctcatcctctttgagc

SEQ ID NO: 261 - DNA, 24 bp, Artificial Sequence
  Description of Artificial Sequence Synthetic oligonucleotide
ccatcaggtggatcaaagtgtctg

SEQ ID NO: 262 - DNA, 30 bp, Artificial Sequence
  Description of Artificial Sequence Synthetic oligonucleotide
accatatccaccgagtcctggacaagatca

SEQ ID NO: 263 - DNA, 25 bp, Artificial Sequence
  Description of Artificial Sequence Synthetic oligonucleotide
cattcaggagtgtacacatttctgc

SEQ ID NO: 264 - DNA, 25 bp, Artificial Sequence
  Description of Artificial Sequence Synthetic oligonucleotide
cattcaggagtgtacacatttctac

SEQ ID NO: 265 - DNA, 25 bp, Artificial Sequence
  Description of Artificial Sequence Synthetic oligonucleotide
cattcaggagtgtacacatttctcc

SEQ ID NO: 266 - DNA, 25 bp, Artificial Sequence
  Description of Artificial Sequence Synthetic oligonucleotide
cattcaggagtgtacacatttcttc

SEQ ID NO: 267 - DNA, 25 bp, Artificial Sequence
  Description of Artificial Sequence Synthetic oligonucleotide
cattcaggagtgtacacatttcagc

SEQ ID NO: 268 - DNA, 25 bp, Artificial Sequence
  Description of Artificial Sequence Synthetic oligonucleotide
cattcaggagtgtacacatttccgc

SEQ ID NO: 269 - DNA, 25 bp, Artificial Sequence
  Description of Artificial Sequence Synthetic oligonucleotide
cattcaggagtgtacacatttcggc

SEQ ID NO: 270 - DNA, 25 bp, Artificial Sequence
  Description of Artificial Sequence Synthetic oligonucleotide
cattcaggagtgtacacatttatgc

SEQ ID NO: 271 - DNA, 25 bp, Artificial Sequence
  Description of Artificial Sequence Synthetic oligonucleotide
cattcaggagtgtacacatttgtgc

SEQ ID NO: 272 - DNA, 25 bp, Artificial Sequence
  Description of Artificial Sequence Synthetic oligonucleotide
cattcaggagtgtacacatttttgc

SEQ ID NO: 273 - DNA, 30 bp, Artificial Sequence
  Description of Artificial Sequence Synthetic oligonucleotide
tgtgtacactcctgaatgcgcagagagaga

SEQ ID NO: 274 - DNA, 18 bp, Artificial Sequence
  Description of Artificial Sequence Synthetic oligonucleotide
agacttcagggtgctggg

SEQ ID NO: 275 - DNA, 19 bp, Artificial Sequence
  Description of Artificial Sequence Synthetic oligonucleotide
cagacttcagggtgctgag

SEQ ID NO: 276 - DNA, 18 bp, Artificial Sequence
  Description of Artificial Sequence Synthetic oligonucleotide
agacttcagggtgctgcg

SEQ ID NO: 277 - DNA, 18 bp, Artificial Sequence
  Description of Artificial Sequence Synthetic oligonucleotide
agacttcagggtgctgtg

SEQ ID NO: 278 - DNA, 18 bp, Artificial Sequence
  Description of Artificial Sequence Synthetic oligonucleotide
agacttcagggtgctagg

SEQ ID NO: 279 - DNA, 18 bp, Artificial Sequence
  Description of Artificial Sequence Synthetic oligonucleotide
agacttcagggtgctcgg

SEQ ID NO: 280 - DNA, 18 bp, Artificial Sequence
  Description of Artificial Sequence Synthetic oligonucleotide
agacttcagggtgcttgg

SEQ ID NO: 281 - DNA, 18 bp, Artificial Sequence
  Description of Artificial Sequence Synthetic oligonucleotide
agacttcagggtgcaggg

SEQ ID NO: 282 - DNA, 18 bp, Artificial Sequence
  Description of Artificial Sequence Synthetic oligonucleotide
agacttcagggtgccggg

SEQ ID NO: 283 - DNA, 18 bp, Artificial Sequence
  Description of Artificial Sequence Synthetic oligonucleotide
agacttcagggtgcgggg

SEQ ID NO: 284 - DNA, 21 bp, Artificial Sequence
  Description of Artificial Sequence Synthetic oligonucleotide
gtcttgtggaagattttctgt

SEQ ID NO: 285 - DNA, 20 bp, Artificial Sequence
  Description of Artificial Sequence Synthetic oligonucleotide
ttgaggcacacaaactcctc

SEQ ID NO: 286 - DNA, 30 bp, Artificial Sequence
  Description of Artificial Sequence Synthetic oligonucleotide
tggctacatcatctcggttccgcatgatga

SEQ ID NO: 287 - DNA, 21 bp, Artificial Sequence
  Description of Artificial Sequence Synthetic oligonucleotide
atgtgtagagggcatggagat

SEQ ID NO: 288 - DNA, 21 bp, Artificial Sequence
  Description of Artificial Sequence Synthetic oligonucleotide
atgtgtagagggcatggagct

SEQ ID NO: 289 - DNA, 21 bp, Artificial Sequence
  Description of Artificial Sequence Synthetic oligonucleotide
atgtgtagagggcatggaggt

SEQ ID NO: 290 - DNA, 21 bp, Artificial Sequence
  Description of Artificial Sequence Synthetic oligonucleotide
atgtgtagagggcatggagtt

SEQ ID NO: 291 - DNA, 21 bp, Artificial Sequence
  Description of Artificial Sequence Synthetic oligonucleotide
atgtgtagagggcatggaaat

SEQ ID NO: 292 - DNA, 21 bp, Artificial Sequence
  Description of Artificial Sequence Synthetic oligonucleotide
atgtgtagagggcatggacat

SEQ ID NO: 293 - DNA, 21 bp, Artificial Sequence
  Description of Artificial Sequence Synthetic oligonucleotide
atgtgtagagggcatggatat

SEQ ID NO: 294 - DNA, 21 bp, Artificial Sequence
  Description of Artificial Sequence Synthetic oligonucleotide
atgtgtagagggcatggcgat

SEQ ID NO: 295 - DNA, 21 bp, Artificial Sequence
  Description of Artificial Sequence Synthetic oligonucleotide
atgtgtagagggcatggggat

SEQ ID NO: 296 - DNA, 21 bp, Artificial Sequence
  Description of Artificial Sequence Synthetic oligonucleotide
atgtgtagagggcatggtgat

SEQ ID NO: 297 - DNA, 23 bp, Artificial Sequence
  Description of Artificial Sequence Synthetic oligonucleotide
  modified_base (13)..(13): g or deoxyinosine
aaatgtgtagagngcatggcgat

SEQ ID NO: 298 - DNA, 23 bp, Artificial Sequence
  Description of Artificial Sequence Synthetic oligonucleotide
aaatgtgtagagggcatggtgat

SEQ ID NO: 299 - DNA, 23 bp, Artificial Sequence
  Description of Artificial Sequence Synthetic oligonucleotide
aaatgtgtagagggcatgtagat

SEQ ID NO: 300 - DNA, 23 bp, Artificial Sequence
  Description of Artificial Sequence Synthetic oligonucleotide
aaatgtgtagagggcatgaagat

SEQ ID NO: 301 - DNA, 29 bp, Artificial Sequence
  Description of Artificial Sequence Synthetic oligonucleotide
cctctacacattttccctggttcctatga

SEQ ID NO: 302 - DNA, 22 bp, Artificial Sequence
  Description of Artificial Sequence Synthetic oligonucleotide
gcagcatgtcgaagatctccat

SEQ ID NO: 303 - DNA, 22 bp, Artificial Sequence
  Description of Artificial Sequence Synthetic oligonucleotide
gcagcatgtcgaagatctccct

SEQ ID NO: 304 - DNA, 22 bp, Artificial Sequence
  Description of Artificial Sequence Synthetic oligonucleotide
gcagcatgtcgaagatctccgt

SEQ ID NO: 305 - DNA, 22 bp, Artificial Sequence
  Description of Artificial Sequence Synthetic oligonucleotide
gcagcatgtcgaagatctcctt

SEQ ID NO: 306 - DNA, 22 bp, Artificial Sequence
  Description of Artificial Sequence Synthetic oligonucleotide
gcagcatgtcgaagatctcaat

SEQ ID NO: 307 - DNA, 22 bp, Artificial Sequence
  Description of Artificial Sequence Synthetic oligonucleotide
gcagcatgtcgaagatctcgat

SEQ ID NO: 308 - DNA, 22 bp, Artificial Sequence
  Description of Artificial Sequence Synthetic oligonucleotide
gcagcatgtcgaagatctctat

SEQ ID NO: 309 - DNA, 22 bp, Artificial Sequence
  Description of Artificial Sequence Synthetic oligonucleotide
gcagcatgtcgaagatctacat

SEQ ID NO: 310 - DNA, 22 bp, Artificial Sequence
  Description of Artificial Sequence Synthetic oligonucleotide
gcagcatgtcgaagatctgcat

SEQ ID NO: 311 - DNA, 22 bp, Artificial Sequence
  Description of Artificial Sequence Synthetic oligonucleotide
gcagcatgtcgaagatcttcat

SEQ ID NO: 312 - DNA, 22 bp, Artificial Sequence
  Description of Artificial Sequence Synthetic oligonucleotide
gtagtcctttctgtgtcttccc

SEQ ID NO: 313 - DNA, 22 bp, Artificial Sequence
  Description of Artificial Sequence Synthetic oligonucleotide
ctttctgtgtcttcccacctac

SEQ ID NO: 314 - DNA, 24 bp, Artificial Sequence
  Description of Artificial Sequence Synthetic oligonucleotide
tgtcttcccacctacagtaacaaa

SEQ ID NO: 315 - DNA, 27 bp, Artificial Sequence
  Description of Artificial Sequence Synthetic oligonucleotide
ctctaaagtagtcctttctgtgtcttc

SEQ ID NO: 316 - DNA, 26 bp, Artificial Sequence
  Description of Artificial Sequence Synthetic oligonucleotide
tctaaagtagtcctttctgtgtcttc

SEQ ID NO: 317 - DNA, 25 bp, Artificial Sequence
  Description of Artificial Sequence Synthetic oligonucleotide
taaagtagtcctttctgtgtcttcc

SEQ ID NO: 318 - DNA, 22 bp, Artificial Sequence
  Description of Artificial Sequence Synthetic oligonucleotide
agtagtcctttctgtgtcttcc

SEQ ID NO: 319 - DNA, 17 bp, Artificial Sequence
  Description of Artificial Sequence Synthetic oligonucleotide
gctagtgggcgcatgta

SEQ ID NO: 320 - DNA, 16 bp, Artificial Sequence
  Description of Artificial Sequence Synthetic oligonucleotide
ctagtgggcgcatgta

SEQ ID NO: 321 - DNA, 28 bp, Artificial Sequence
  Description of Artificial Sequence Synthetic oligonucleotide
tctatgacctgctgctggagatgctgga

SEQ ID NO: 322 - DNA, 21 bp, Artificial Sequence
  Description of Artificial Sequence Synthetic oligonucleotide
acagcatgaagtgcaagaaca

SEQ ID NO: 323 - DNA, 21 bp, Artificial Sequence
  Description of Artificial Sequence Synthetic oligonucleotide
acagcatgaagtgcaagaaaa

SEQ ID NO: 324 - DNA, 21 bp, Artificial Sequence
  Description of Artificial Sequence Synthetic oligonucleotide
acagcatgaagtgcaagaaga

SEQ ID NO: 325 - DNA, 21 bp, Artificial Sequence
  Description of Artificial Sequence Synthetic oligonucleotide
acagcatgaagtgcaagaata

SEQ ID NO: 326 - DNA, 21 bp, Artificial Sequence
  Description of Artificial Sequence Synthetic oligonucleotide
acagcatgaagtgcaagacca

SEQ ID NO: 327 - DNA, 21 bp, Artificial Sequence
  Description of Artificial Sequence Synthetic oligonucleotide
acagcatgaagtgcaagagca

SEQ ID NO: 328 - DNA, 21 bp, Artificial Sequence
  Description of Artificial Sequence Synthetic oligonucleotide
acagcatgaagtgcaagatca

SEQ ID NO: 329 - DNA, 21 bp, Artificial Sequence
  Description of Artificial Sequence Synthetic oligonucleotide
acagcatgaagtgcaagcaca

SEQ ID NO: 330 - DNA, 21 bp, Artificial Sequence
  Description of Artificial Sequence Synthetic oligonucleotide
acagcatgaagtgcaaggaca

SEQ ID NO: 331 - DNA, 21 bp, Artificial Sequence
  Description of Artificial Sequence Synthetic oligonucleotide
acagcatgaagtgcaagtaca

SEQ ID NO: 332 - DNA, 24 bp, Artificial Sequence
  Description of Artificial Sequence Synthetic oligonucleotide
tgtacagcatgaagtgcaagcaca

SEQ ID NO: 333 - DNA, 24 bp, Artificial Sequence
  Description of Artificial Sequence Synthetic oligonucleotide
tgtacagcatgaagtgcaaggaca

SEQ ID NO: 334 - DNA, 30 bp, Artificial Sequence; identical to SEQ ID NO: 95
  Description of Artificial Sequence Synthetic oligonucleotide

SEQ ID NO: 335 - DNA, 30 bp, Artificial Sequence; identical to SEQ ID NO: 95
  Description of Artificial Sequence Synthetic oligonucleotide

SEQ ID NO: 336 - DNA, 19 bp, Artificial Sequence
  Description of Artificial Sequence Synthetic oligonucleotide
ggtcatagaggggcaccat

SEQ ID NO: 337 - DNA, 19 bp, Artificial Sequence
  Description of Artificial Sequence Synthetic oligonucleotide
ggtcatagaggggcaccat

SEQ ID NO: 338 - DNA, 19 bp, Artificial Sequence
  Description of Artificial Sequence Synthetic oligonucleotide
ggtcatagaggggcaccct

SEQ ID NO: 339 - DNA, 19 bp, Artificial Sequence
  Description of Artificial Sequence Synthetic oligonucleotide
ggtcatagaggggcaccmt

SEQ ID NO: 340 - DNA, 19 bp, Artificial Sequence
  Description of Artificial Sequence Synthetic oligonucleotide
ggtcatagaggggcaccgt

SEQ ID NO: 341 - DNA, 19 bp, Artificial Sequence
  Description of Artificial Sequence Synthetic oligonucleotide
ggtcatagaggggcacctt

SEQ ID NO: 342 - DNA, 19 bp, Artificial Sequence
  Description of Artificial Sequence Synthetic oligonucleotide
ggtcatagaggggcacaat

SEQ ID NO: 343 - DNA, 19 bp, Artificial Sequence
  Description of Artificial Sequence Synthetic oligonucleotide
ggtcatagaggggcacgat

SEQ ID NO: 344 - DNA, 19 bp, Artificial Sequence
  Description of Artificial Sequence Synthetic oligonucleotide
ggtcatagaggggcactat

SEQ ID NO: 345 - DNA, 19 bp, Artificial Sequence
  Description of Artificial Sequence Synthetic oligonucleotide
ggtcatagaggggcaacat

SEQ ID NO: 346 - DNA, 19 bp, Artificial Sequence
  Description of Artificial Sequence Synthetic oligonucleotide
ggtcatagaggggcagcat

SEQ ID NO: 347 - DNA, 19 bp, Artificial Sequence
  Description of Artificial Sequence Synthetic oligonucleotide
ggtcatagaggggcatcat

SEQ ID NO: 348 - DNA, 20 bp, Artificial Sequence
  Description of Artificial Sequence Synthetic oligonucleotide
aggtcatagaggggcagcat

SEQ ID NO: 349 - DNA, 20 bp, Artificial Sequence
  Description of Artificial Sequence Synthetic oligonucleotide
aggtcatagaggggctccat

SEQ ID NO: 350 - DNA, 20 bp, Artificial Sequence
  Description of Artificial Sequence Synthetic oligonucleotide
aggtcatagaggggcgccat

SEQ ID NO: 351 - DNA, 20 bp, Artificial Sequence
  Description of Artificial Sequence Synthetic oligonucleotide
aggtcatagaggggccccat

SEQ ID NO: 352 - DNA, 21 bp, Artificial Sequence
  Description of Artificial Sequence Synthetic oligonucleotide
caggtcatagaggggctccat

SEQ ID NO: 353 - DNA, 21 bp, Artificial Sequence
  Description of Artificial Sequence Synthetic oligonucleotide
caggtcatagaggggcgccat

SEQ ID NO: 354 - DNA, 21 bp, Artificial Sequence
  Description of Artificial Sequence Synthetic oligonucleotide
caggtcatagaggggccccat

SEQ ID NO: 355 - DNA, 21 bp, Artificial Sequence
  Description of Artificial Sequence Synthetic oligonucleotide
acagcatgaagtgcaagaaca

SEQ ID NO: 356 - DNA, 21 bp, Artificial Sequence
  Description of Artificial Sequence Synthetic oligonucleotide
acagcatgaagtgcaagaaca

SEQ ID NO: 357 - DNA, 22 bp, Artificial Sequence
  Description of Artificial Sequence Synthetic oligonucleotide
ctttctgtgtcttcccacctac

SEQ ID NO: 358 - DNA, 17 bp, Artificial Sequence
  Description of Artificial Sequence Synthetic oligonucleotide
gctttggtccgtctcct

SEQ ID NO: 359 - DNA, 19 bp, Artificial Sequence
  Description of Artificial Sequence Synthetic oligonucleotide
tggctttggtccgtctcct

SEQ ID NO: 360 - DNA, 18 bp, Artificial Sequence
  Description of Artificial Sequence Synthetic oligonucleotide
atgtaggcggtgggcgtc

SEQ ID NO: 361 - DNA, 18 bp, Artificial Sequence
  Description of Artificial Sequence Synthetic oligonucleotide
ctccacggctagtgggcg

SEQ ID NO: 362 - DNA, 18 bp, Artificial Sequence
  Description of Artificial Sequence Synthetic oligonucleotide
tgcccctccacggctagt

SEQ ID NO: 363 - DNA, 28 bp, Artificial Sequence
  Description of Artificial Sequence Synthetic oligonucleotide
tctatgacctgctgctggagatgctgga

SEQ ID NO: 364 - DNA, 22 bp, Artificial Sequence
  Description of Artificial Sequence Synthetic oligonucleotide
gcatgaagtgcaagaacgtgga

SEQ ID NO: 365 - DNA, 22 bp, Artificial Sequence
  Description of Artificial Sequence Synthetic oligonucleotide
gcatgaagtgcaagaacgtgaa

SEQ ID NO: 366 - DNA, 22 bp, Artificial Sequence
  Description of Artificial Sequence Synthetic oligonucleotide
gcatgaagtgcaagaacgtgca

SEQ ID NO: 367 - DNA, 22 bp, Artificial Sequence
  Description of Artificial Sequence Synthetic oligonucleotide
gcatgaagtgcaagaacgtgta

SEQ ID NO: 368 - DNA, 22 bp, Artificial Sequence
  Description of Artificial Sequence Synthetic oligonucleotide
gcatgaagtgcaagaacgtaga

SEQ ID NO: 369 - DNA, 22 bp, Artificial Sequence
  Description of Artificial Sequence Synthetic oligonucleotide
gcatgaagtgcaagaacgtcga

SEQ ID NO: 370 - DNA, 22 bp, Artificial Sequence
  Description of Artificial Sequence Synthetic oligonucleotide
gcatgaagtgcaagaacgttga

SEQ ID NO: 371 - DNA, 22 bp, Artificial Sequence
  Description of Artificial Sequence Synthetic oligonucleotide
gcatgaagtgcaagaacgagga

SEQ ID NO: 372 - DNA, 22 bp, Artificial Sequence
  Description of Artificial Sequence Synthetic oligonucleotide
gcatgaagtgcaagaacgcgga

SEQ ID NO: 373 - DNA, 22 bp, Artificial Sequence
  Description of Artificial Sequence Synthetic oligonucleotide
gcatgaagtgcaagaacgggga

SEQ ID NO: 374 - DNA, 29 bp, Artificial Sequence
  Description of Artificial Sequence Synthetic oligonucleotide
tgcacttcatgctgtacagatgctccatg

SEQ ID NO: 375 - DNA, 35 bp, Artificial Sequence
  Description of Artificial Sequence Synthetic oligonucleotide
  Description of Combined DNA/RNA Molecule Synthetic oligonucleotide
tgcacttcauuugctguuuacagatgcuuuccatg

SEQ ID NO: 376 - DNA, 28 bp, Artificial Sequence
  Description of Artificial Sequence Synthetic oligonucleotide
tgcacttcatgctgtacagatgctccat

SEQ ID NO: 377 - DNA, 40 bp, Artificial Sequence
  Description of Artificial Sequence Synthetic oligonucleotide
  Description of Combined DNA/RNA Molecule Synthetic oligonucleotide
tgcacttcauuuugctguuuuacagauuuugctccauuuu

SEQ ID NO: 378 - DNA, 18 bp, Artificial Sequence
  Description of Artificial Sequence Synthetic oligonucleotide
gcaggtcatagaggggct

SEQ ID NO: 379 - DNA, 18 bp, Artificial Sequence
  Description of Artificial Sequence Synthetic oligonucleotide
gcaggtcatagaggggat

SEQ ID NO: 380 - DNA, 18 bp, Artificial Sequence
  Description of Artificial Sequence Synthetic oligonucleotide
gcaggtcatagagggggt

SEQ ID NO: 381 - DNA, 18 bp, Artificial Sequence
  Description of Artificial Sequence Synthetic oligonucleotide
gcaggtcatagaggggtt

SEQ ID NO: 382 - DNA, 18 bp, Artificial Sequence
  Description of Artificial Sequence Synthetic oligonucleotide
gcaggtcatagagggact

SEQ ID NO: 383 - DNA, 18 bp, Artificial Sequence
  Description of Artificial Sequence Synthetic oligonucleotide
gcaggtcatagagggcct

SEQ ID NO: 384 - DNA, 18 bp, Artificial Sequence
  Description of Artificial Sequence Synthetic oligonucleotide
gcaggtcatagagggtct

SEQ ID NO: 385 - DNA, 18 bp, Artificial Sequence
  Description of Artificial Sequence Synthetic oligonucleotide
gcaggtcatagaggagct

SEQ ID NO: 386 - DNA, 18 bp, Artificial Sequence
  Description of Artificial Sequence Synthetic oligonucleotide
gcaggtcatagaggcgct

SEQ ID NO: 387 - DNA, 18 bp, Artificial Sequence
  Description of Artificial Sequence Synthetic oligonucleotide
gcaggtcatagaggtgct

SEQ ID NO: 388 - DNA, 18 bp, Artificial Sequence
  Description of Artificial Sequence Synthetic oligonucleotide
gcaggtcatagaggggct

SEQ ID NO: 389 - DNA, 18 bp, Artificial Sequence
  Description of Artificial Sequence Synthetic oligonucleotide
gcaggtcatagagtggct

SEQ ID NO: 390 - DNA, 18 bp, Artificial Sequence
  Description of Artificial Sequence Synthetic oligonucleotide
gcaggtcatagagcggct

SEQ ID NO: 391 - DNA, 20 bp, Artificial Sequence
  Description of Artificial Sequence Synthetic oligonucleotide
cagcaggtcatagagaggct

SEQ ID NO: 392 - DNA, 20 bp, Artificial Sequence
  Description of Artificial Sequence Synthetic oligonucleotide
cagcaggtcatagagtggct

SEQ ID NO: 393 - DNA, 20 bp, Artificial Sequence
  Description of Artificial Sequence Synthetic oligonucleotide
cagcaggtcatagagcggct

SEQ ID NO: 394 - DNA, 22 bp, Artificial Sequence
  Description of Artificial Sequence Synthetic oligonucleotide
ctgtgtcttcccacctacagta

SEQ ID NO: 395 - DNA, 17 bp, Artificial Sequence
  Description of Artificial Sequence Synthetic oligonucleotide
aagtggctttggtccgt

SEQ ID NO: 396 - DNA, 23 bp, Artificial Sequence
  Description of Artificial Sequence Synthetic oligonucleotide
tacatgcgcccactagccgtgga

SEQ ID NO: 397 - DNA, 16 bp, Artificial Sequence
  Description of Artificial Sequence Synthetic oligonucleotide
acgtggtgcccctctg

SEQ ID NO: 398 - DNA, 16 bp, Artificial Sequence
  Description of Artificial Sequence Synthetic oligonucleotide
acgtggtgcccctcag

SEQ ID NO: 399 - DNA, 16 bp, Artificial Sequence
  Description of Artificial Sequence Synthetic oligonucleotide
acgtggtgcccctccg

SEQ ID NO: 400 - DNA, 16 bp, Artificial Sequence
  Description of Artificial Sequence Synthetic oligonucleotide
acgtggtgcccctcgg

SEQ ID NO: 401 - DNA, 16 bp, Artificial Sequence
  Description of Artificial Sequence Synthetic oligonucleotide
acgtggtgcccctatg

SEQ ID NO: 402 - DNA, 16 bp, Artificial Sequence
  Description of Artificial Sequence Synthetic oligonucleotide
acgtggtgcccctgtg

SEQ ID NO: 403 - DNA, 16 bp, Artificial Sequence
  Description of Artificial Sequence Synthetic oligonucleotide
acgtggtgcccctttg

SEQ ID NO: 404 - DNA, 16 bp, Artificial Sequence
  Description of Artificial Sequence Synthetic oligonucleotide
acgtggtgccccactg

SEQ ID NO: 405 - DNA, 16 bp, Artificial Sequence
  Description of Artificial Sequence Synthetic oligonucleotide
acgtggtgcccccctg

SEQ ID NO: 406 - DNA, 16 bp, Artificial Sequence
  Description of Artificial Sequence Synthetic oligonucleotide
acgtggtgccccgctg

SEQ ID NO: 407 - DNA, 30 bp, Artificial Sequence; identical to SEQ ID NO: 95
  Description of Artificial Sequence Synthetic oligonucleotide

SEQ ID NO: 408 - DNA, 17 bp, Artificial Sequence
  Description of Artificial Sequence Synthetic oligonucleotide
ctccagcagcaggtcac

SEQ ID NO: 409 - DNA, 17 bp, Artificial Sequence
  Description of Artificial Sequence Synthetic oligonucleotide
ctccagcagcaggtccc

SEQ ID NO: 410 - DNA, 17 bp, Artificial Sequence
  Description of Artificial Sequence Synthetic oligonucleotide
ctccagcagcaggtcgc

SEQ ID NO: 411 - DNA, 17 bp, Artificial Sequence
  Description of Artificial Sequence Synthetic oligonucleotide
ctccagcagcaggtctc

SEQ ID NO: 412 - DNA, 17 bp, Artificial Sequence
  Description of Artificial Sequence Synthetic oligonucleotide
ctccagcagcaggtaac

SEQ ID NO: 413 - DNA, 17 bp, Artificial Sequence
  Description of Artificial Sequence Synthetic oligonucleotide
ctccagcagcaggtgac

SEQ ID NO: 414 - DNA, 17 bp, Artificial Sequence
  Description of Artificial Sequence Synthetic oligonucleotide
ctccagcagcaggttac

SEQ ID NO: 415 - DNA, 17 bp, Artificial Sequence
  Description of Artificial Sequence Synthetic oligonucleotide
ctccagcagcaggacac

SEQ ID NO: 416 - DNA, 17 bp, Artificial Sequence
  Description of Artificial Sequence Synthetic oligonucleotide
ctccagcagcaggccac

SEQ ID NO: 417 - DNA, 17 bp, Artificial Sequence
  Description of Artificial Sequence Synthetic oligonucleotide
ctccagcagcagggcac

SEQ ID NO: 418 - DNA, 19 bp, Artificial Sequence
  Description of Artificial Sequence Synthetic oligonucleotide
atctccagcagcaggacac

SEQ ID NO: 419 - DNA, 20 bp, Artificial Sequence
  Description of Artificial Sequence Synthetic oligonucleotide
catctccagcagcaggacac

SEQ ID NO: 420 - DNA, 19 bp, Artificial Sequence
  Description of Artificial Sequence Synthetic oligonucleotide
atctccagcagcaggtcac

SEQ ID NO: 421 - DNA, 19 bp, Artificial Sequence
  Description of Artificial Sequence Synthetic oligonucleotide
atctccagcagcaggtcac

SEQ ID NO: 422 - DNA, 19 bp, Artificial Sequence
  Description of Artificial Sequence Synthetic oligonucleotide
atctccagcagcaggtcac

SEQ ID NO: 423 - DNA, 17 bp, Artificial Sequence
  Description of Artificial Sequence Synthetic oligonucleotide
ctccagcagcaggtcac

SEQ ID NO: 424 - DNA, 17 bp, Artificial Sequence
  Description of Artificial Sequence Synthetic oligonucleotide
ctccagcagcaggtcac

SEQ ID NO: 425 - DNA, 17 bp, Artificial Sequence
  Description of Artificial Sequence Synthetic oligonucleotide
ctccagcagcaggtcac

SEQ ID NO: 426 - DNA, 16 bp, Artificial Sequence
  Description of Artificial Sequence Synthetic oligonucleotide
aacgtggtgcccctca

SEQ ID NO: 427 - DNA, 16 bp, Artificial Sequence
  Description of Artificial Sequence Synthetic oligonucleotide
aacgtggtgcccctaa

SEQ ID NO: 428 - DNA, 16 bp, Artificial Sequence
  Description of Artificial Sequence Synthetic oligonucleotide
aacgtggtgcccctga

SEQ ID NO: 429 - DNA, 16 bp, Artificial Sequence
  Description of Artificial Sequence Synthetic oligonucleotide
aacgtggtgcccctta

SEQ ID NO: 430 - DNA, 16 bp, Artificial Sequence
  Description of Artificial Sequence Synthetic oligonucleotide
aacgtggtgccccaca

SEQ ID NO: 431 - DNA, 16 bp, Artificial Sequence
  Description of Artificial Sequence Synthetic oligonucleotide
aacgtggtgcccccca

SEQ ID NO: 432 - DNA, 16 bp, Artificial Sequence
  Description of Artificial Sequence Synthetic oligonucleotide
aacgtggtgccccgca

SEQ ID NO: 433 - DNA, 16 bp, Artificial Sequence
  Description of Artificial Sequence Synthetic oligonucleotide
aacgtggtgcccatca

SEQ ID NO: 434 - DNA, 16 bp, Artificial Sequence
  Description of Artificial Sequence Synthetic oligonucleotide
aacgtggtgcccgtca

SEQ ID NO: 435 - DNA, 16 bp, Artificial Sequence
  Description of Artificial Sequence Synthetic oligonucleotide
aacgtggtgcccttca

SEQ ID NO: 436 - DNA, 18 bp, Artificial Sequence
  Description of Artificial Sequence Synthetic oligonucleotide
ctccagcagcaggtcatt

SEQ ID NO: 437 - DNA, 18 bp, Artificial Sequence
  Description of Artificial Sequence Synthetic oligonucleotide
ctccagcagcaggtcaat

SEQ ID NO: 438 - DNA, 18 bp, Artificial Sequence
  Description of Artificial Sequence Synthetic oligonucleotide
ctccagcagcaggtcact

SEQ ID NO: 439 - DNA, 18 bp, Artificial Sequence
  Description of Artificial Sequence Synthetic oligonucleotide
ctccagcagcaggtcagt

SEQ ID NO: 440 - DNA, 18 bp, Artificial Sequence
  Description of Artificial Sequence Synthetic oligonucleotide
ctccagcagcaggtcctt

SEQ ID NO: 441 - DNA, 18 bp, Artificial Sequence
  Description of Artificial Sequence Synthetic oligonucleotide
ctccagcagcaggtcgtt

SEQ ID NO: 442 - DNA, 18 bp, Artificial Sequence
  Description of Artificial Sequence Synthetic oligonucleotide
ctccagcagcaggtcttt

SEQ ID NO: 443 - DNA, 18 bp, Artificial Sequence
  Description of Artificial Sequence Synthetic oligonucleotide
ctccagcagcaggtaatt

SEQ ID NO: 444 - DNA, 18 bp, Artificial Sequence
  Description of Artificial Sequence Synthetic oligonucleotide
ctccagcagcaggtgatt

SEQ ID NO: 445 - DNA, 18 bp, Artificial Sequence
  Description of Artificial Sequence Synthetic oligonucleotide
ctccagcagcaggttatt

SEQ ID NO: 446 - DNA, 20 bp, Artificial Sequence
  Description of Artificial Sequence Synthetic oligonucleotide
atctccagcagcaggttatt

SEQ ID NO: 447 - DNA, 21 bp, Artificial Sequence
  Description of Artificial Sequence Synthetic oligonucleotide
catctccagcagcaggttatt

SEQ ID NO: 448 - DNA, 22 bp, Artificial Sequence
  Description of Artificial Sequence Synthetic oligonucleotide
gcatctccagcagcaggttatt

SEQ ID NO: 449 - DNA, 16 bp, Artificial Sequence
  Description of Artificial Sequence Synthetic oligonucleotide
acgtggtgcccctctc

SEQ ID NO: 450 - DNA, 16 bp, Artificial Sequence
  Description of Artificial Sequence Synthetic oligonucleotide
acgtggtgcccctcac

SEQ ID NO: 451 - DNA, 16 bp, Artificial Sequence
  Description of Artificial Sequence Synthetic oligonucleotide
acgtggtgcccctccc

SEQ ID NO: 452 - DNA, 16 bp, Artificial Sequence
  Description of Artificial Sequence Synthetic oligonucleotide
acgtggtgcccctcgc

SEQ ID NO: 453 - DNA, 16 bp, Artificial Sequence
  Description of Artificial Sequence Synthetic oligonucleotide
acgtggtgcccctatc

SEQ ID NO: 454 - DNA, 16 bp, Artificial Sequence
  Description of Artificial Sequence Synthetic oligonucleotide
acgtggtgcccctgtc

SEQ ID NO: 455 - DNA, 16 bp, Artificial Sequence
  Description of Artificial Sequence Synthetic oligonucleotide
acgtggtgcccctttc

SEQ ID NO: 456 - DNA, 16 bp, Artificial Sequence
  Description of Artificial Sequence Synthetic oligonucleotide
acgtggtgccccactc

SEQ ID NO: 457 - DNA, 16 bp, Artificial Sequence
  Description of Artificial Sequence Synthetic oligonucleotide
acgtggtgcccccctc

SEQ ID NO: 458 - DNA, 16 bp, Artificial Sequence
  Description of Artificial Sequence Synthetic oligonucleotide
acgtggtgccccgctc

SEQ ID NO: 459 - DNA, 19 bp, Artificial Sequence
  Description of Artificial Sequence Synthetic oligonucleotide
atctccagcagcaggtcag

SEQ ID NO: 460 - DNA, 19 bp, Artificial Sequence
  Description of Artificial Sequence Synthetic oligonucleotide
atctccagcagcaggtccg

SEQ ID NO: 461 - DNA, 19 bp, Artificial Sequence
  Description of Artificial Sequence Synthetic oligonucleotide
atctccagcagcaggtcgg

SEQ ID NO: 462 - DNA, 19 bp, Artificial Sequence
  Description of Artificial Sequence Synthetic oligonucleotide
atctccagcagcaggtctg

SEQ ID NO: 463 - DNA, 19 bp, Artificial Sequence
  Description of Artificial Sequence Synthetic oligonucleotide
atctccagcagcaggtaag

SEQ ID NO: 464 - DNA, 19 bp, Artificial Sequence
  Description of Artificial Sequence Synthetic oligonucleotide
atctccagcagcaggtgag

SEQ ID NO: 465 - DNA, 19 bp, Artificial Sequence
  Description of Artificial Sequence Synthetic oligonucleotide
atctccagcagcaggttag

SEQ ID NO: 466 - DNA, 19 bp, Artificial Sequence
  Description of Artificial Sequence Synthetic oligonucleotide
atctccagcagcaggacag

SEQ ID NO: 467 - DNA, 19 bp, Artificial Sequence
  Description of Artificial Sequence Synthetic oligonucleotide
atctccagcagcaggccag

SEQ ID NO: 468 - DNA, 19 bp, Artificial Sequence
  Description of Artificial Sequence Synthetic oligonucleotide
atctccagcagcagggcag

SEQ ID NO: 469 - DNA, 20 bp, Artificial Sequence
  Description of Artificial Sequence Synthetic oligonucleotide
catctccagcagcaggacag

SEQ ID NO: 470 - DNA, 20 bp, Artificial Sequence
  Description of Artificial Sequence Synthetic oligonucleotide
catctccagcagcagggcag

SEQ ID NO: 471 - DNA, 21 bp, Artificial Sequence
  Description of Artificial Sequence Synthetic oligonucleotide
gcatctccagcagcaggacag

SEQ ID NO: 472 - DNA, 17 bp, Artificial Sequence
  Description of Artificial Sequence Synthetic oligonucleotide
ctccagcagcagggcag

SEQ ID NO: 473 - DNA, 19 bp, Artificial Sequence
  Description of Artificial Sequence Synthetic oligonucleotide
agagatgatggggagggca

SEQ ID NO: 474 - DNA, 17 bp, Artificial Sequence
  Description of Artificial Sequence Synthetic oligonucleotide
agatgatggggagggca

SEQ ID NO: 475 - DNA, 18 bp, Artificial Sequence
  Description of Artificial Sequence Synthetic oligonucleotide
tcagcatccaacaaggca

SEQ ID NO: 476 - DNA, 19 bp, Artificial Sequence
  Description of Artificial Sequence Synthetic oligonucleotide
ctcagcatccaacaaggca

SEQ ID NO: 477 - DNA, 28 bp, Artificial Sequence
  Description of Artificial Sequence Synthetic oligonucleotide
ttgtccctgacggccgaccagatggtca

SEQ ID NO: 478 - DNA, 25 bp, Artificial Sequence
  Description of Artificial Sequence Synthetic oligonucleotide
cgctcatgatcaaacgctctaagag

SEQ ID NO: 479 - DNA, 25 bp, Artificial Sequence
  Description of Artificial Sequence Synthetic oligonucleotide
cgctcatgatcaaacgctctaagcg

SEQ ID NO: 480 - DNA, 25 bp, Artificial Sequence
  Description of Artificial Sequence Synthetic oligonucleotide
cgctcatgatcaaacgctctaaggg

SEQ ID NO: 481 - DNA, 25 bp, Artificial Sequence
  Description of Artificial Sequence Synthetic oligonucleotide
cgctcatgatcaaacgctctaagtg

SEQ ID NO: 482 - DNA, 25 bp, Artificial Sequence
  Description of Artificial Sequence Synthetic oligonucleotide
cgctcatgatcaaacgctctaaaag

SEQ ID NO: 483 - DNA, 25 bp, Artificial Sequence
  Description of Artificial Sequence Synthetic oligonucleotide
cgctcatgatcaaacgctctaacag

SEQ ID NO: 484 - DNA, 25 bp, Artificial Sequence
  Description of Artificial Sequence Synthetic oligonucleotide
cgctcatgatcaaacgctctaatag

SEQ ID NO: 485 - DNA, 25 bp, Artificial Sequence
  Description of Artificial Sequence Synthetic oligonucleotide
cgctcatgatcaaacgctctacgag

SEQ ID NO: 486 - DNA, 25 bp, Artificial Sequence
  Description of Artificial Sequence Synthetic oligonucleotide
cgctcatgatcaaacgctctaggag

SEQ ID NO: 487 - DNA, 25 bp, Artificial Sequence
  Description of Artificial Sequence Synthetic oligonucleotide
cgctcatgatcaaacgctctatgag

SEQ ID NO: 488 - DNA, 30 bp, Artificial Sequence
  Description of Artificial Sequence Synthetic oligonucleotide
tttgatcatgagcgggcttggccaaaggtt

SEQ ID NO: 489 - DNA, 18 bp, Artificial Sequence
  Description of Artificial Sequence Synthetic oligonucleotide
acaaggccaggctgttcc

SEQ ID NO: 490 - DNA, 18 bp, Artificial Sequence
  Description of Artificial Sequence Synthetic oligonucleotide
acaaggccaggctgttac

SEQ ID NO: 491 - DNA, 18 bp, Artificial Sequence
  Description of Artificial Sequence Synthetic oligonucleotide
acaaggccaggctgttgc

SEQ ID NO: 492 - DNA, 18 bp, Artificial Sequence
  Description of Artificial Sequence Synthetic oligonucleotide
acaaggccaggctgtttc

SEQ ID NO: 493 - DNA, 18 bp, Artificial Sequence
  Description of Artificial Sequence Synthetic oligonucleotide
acaaggccaggctgtacc

SEQ ID NO: 494 - DNA, 18 bp, Artificial Sequence
  Description of Artificial Sequence Synthetic oligonucleotide
acaaggccaggctgtccc

SEQ ID NO: 495 - DNA, 18 bp, Artificial Sequence
  Description of Artificial Sequence Synthetic oligonucleotide
acaaggccaggctgtgcc

SEQ ID NO: 496 - DNA, 18 bp, Artificial Sequence
  Description of Artificial Sequence Synthetic oligonucleotide
acaaggccaggctgatcc

SEQ ID NO: 497 - DNA, 18 bp, Artificial Sequence
  Description of Artificial Sequence Synthetic oligonucleotide
acaaggccaggctgctcc

SEQ ID NO: 498 - DNA, 18 bp, Artificial Sequence
  Description of Artificial Sequence Synthetic oligonucleotide
acaaggccaggctggtcc

SEQ ID NO: 499 - DNA, 19 bp, Artificial Sequence
  Description of Artificial Sequence Synthetic oligonucleotide
gtctggcgagagatgcaaa

SEQ ID NO: 500 - DNA, 28 bp, Artificial Sequence
  Description of Artificial Sequence Synthetic oligonucleotide
ctctactttccttacctccttccttcca

SEQ ID NO: 501 - DNA, 22 bp, Artificial Sequence
  Description of Artificial Sequence Synthetic oligonucleotide
gcctcaatgaagacaacttgaa

SEQ ID NO: 502 - DNA, 22 bp, Artificial Sequence
  Description of Artificial Sequence Synthetic oligonucleotide
aggataaagtggatctgctgca

SEQ ID NO: 503 - DNA, 20 bp, Artificial Sequence
  Description of Artificial Sequence Synthetic oligonucleotide
cctggcgtcgattatctgaa

SEQ ID NO: 504 - DNA, 22 bp, Artificial Sequence
  Description of Artificial Sequence Synthetic oligonucleotide
gctgttaattgtccatgcataa

SEQ ID NO: 505 - DNA, 21 bp, Artificial Sequence
  Description of Artificial Sequence Synthetic oligonucleotide
gaaaggggagaacaagctaaa

SEQ ID NO: 506 - DNA, 21 bp, Artificial Sequence
  Description of Artificial Sequence Synthetic oligonucleotide
gaggaatggatttcaatggaa

SEQ ID NO: 507 - DNA, 21 bp, Artificial Sequence
  Description of Artificial Sequence Synthetic oligonucleotide
ccctgggtctgtgatcactaa

SEQ ID NO: 508 - DNA, 20 bp, Artificial Sequence
  Description of Artificial Sequence Synthetic oligonucleotide
gccacggaccatgaccatga

SEQ ID NO: 509 - DNA, 20 bp, Artificial Sequence
  Description of Artificial Sequence Synthetic oligonucleotide
cttgagctgcggacggttca

SEQ ID NO: 510 - DNA, 25 bp, Artificial Sequence
  Description of Artificial Sequence Synthetic oligonucleotide
tggccctactgcatcagatccaagg

SEQ ID NO: 511 - DNA, 22 bp, Artificial Sequence
  Description of Artificial Sequence Synthetic oligonucleotide
cagagaaagattggccagtacc

SEQ ID NO: 512 - DNA, 23 bp, Artificial Sequence
  Description of Artificial Sequence Synthetic oligonucleotide
gccagtaccaatgacaagggaag

SEQ ID NO: 513 - DNA, 21 bp, Artificial Sequence
  Description of Artificial Sequence Synthetic oligonucleotide
ccagggtggcagagaaagatt

SEQ ID NO: 514 - DNA, 23 bp, Artificial Sequence
  Description of Artificial Sequence Synthetic oligonucleotide
cagactccataatggtagcctga

SEQ ID NO: 515 - DNA, 25 bp, Artificial Sequence
  Description of Artificial Sequence Synthetic oligonucleotide
taatggtagcctgaagcatagtcat

SEQ ID NO: 516 - DNA, 23 bp, Artificial Sequence
  Description of Artificial Sequence Synthetic oligonucleotide
cactgcacagtagcgagtctcct

SEQ ID NO: 517 - DNA, 28 bp, Artificial Sequence
  Description of Artificial Sequence Synthetic oligonucleotide
tgacaagggaagtatggctatggaatct

SEQ ID NO: 518 - DNA, 28 bp, Artificial Sequence
  Description of Artificial Sequence Synthetic oligonucleotide
tgacaagggaagtatggctatggaatct

SEQ ID NO: 519 - DNA, 28 bp, Artificial Sequence
  Description of Artificial Sequence Synthetic oligonucleotide
ctatggaatctgccaaggagactcgcta

SEQ ID NO: 520 - DNA, 28 bp, Artificial Sequence
  Description of Artificial Sequence Synthetic oligonucleotide
tgacaagggaagtatggctatggaatct

SEQ ID NO: 521 - DNA, 28 bp, Artificial Sequence
  Description of Artificial Sequence Synthetic oligonucleotide
cagtaccaatgacaagggaagtatggct

SEQ ID NO: 522 - DNA, 28 bp, Artificial Sequence
  Description of Artificial Sequence Synthetic oligonucleotide
tgacaagggaagtatggctatggaatct

SEQ ID NO: 523 - DNA, 22 bp, Artificial Sequence
  Description of Artificial Sequence Synthetic oligonucleotide
catgatcaggtccaccttctac

SEQ ID NO: 524 - DNA, 23 bp, Artificial Sequence
  Description of Artificial Sequence Synthetic oligonucleotide
ccatgatcaggtccaccttctac

SEQ ID NO: 525 - DNA, 24 bp, Artificial Sequence
  Description of Artificial Sequence Synthetic oligonucleotide
tccatgatcaggtccaccttctac

SEQ ID NO: 526 - DNA, 23 bp, Artificial Sequence
  Description of Artificial Sequence Synthetic oligonucleotide
gagcaagttaggagcaaacagta

SEQ ID NO: 527 - DNA, 24 bp, Artificial Sequence
  Description of Artificial Sequence Synthetic oligonucleotide
agagcaagttaggagcaaacagta

SEQ ID NO: 528 - DNA, 25 bp, Artificial Sequence
  Description of Artificial Sequence Synthetic oligonucleotide
aagagcaagttaggagcaaacagta

SEQ ID NO: 529 - DNA, 22 bp, Artificial Sequence
  Description of Artificial Sequence Synthetic oligonucleotide
catgatcaggtccaccttctag

SEQ ID NO: 530 - DNA, 23 bp, Artificial Sequence
  Description of Artificial Sequence Synthetic oligonucleotide
gagcaagttaggagcaaacagta

SEQ ID NO: 531 - DNA, 24 bp, Artificial Sequence
  Description of Artificial Sequence Synthetic oligonucleotide
agagcaagttaggagcaaacagta

SEQ ID NO: 532 - DNA, 25 bp, Artificial Sequence
  Description of Artificial Sequence Synthetic oligonucleotide
aagagcaagttaggagcaaacagta

SEQ ID NO: 533 - DNA, 28 bp, Artificial Sequence
  Description of Artificial Sequence Synthetic oligonucleotide
tgcctggctagagatcctgatgattggt

SEQ ID NO: 534 - DNA, 23 bp, Artificial Sequence
  Description of Artificial Sequence Synthetic oligonucleotide
ggtagagatcttcgacatgctgg

SEQ ID NO: 535 - DNA, 25 bp, Artificial Sequence
  Description of Artificial Sequence Synthetic oligonucleotide
atggtagagatcttcgacatgctgg

SEQ ID NO: 536 - DNA, 23 bp, Artificial Sequence
  Description of Artificial Sequence Synthetic oligonucleotide
atgtgtagagggcatggagatct

SEQ ID NO: 537 - DNA, 26 bp, Artificial Sequence
  Description of Artificial Sequence Synthetic oligonucleotide
gttatcaactcaccagaattaagcaa

SEQ ID NO: 538 - DNA, 27 bp, Artificial Sequence
  Description of Artificial Sequence Synthetic oligonucleotide
tgttatcaactcaccagaattaagcaa

SEQ ID NO: 539 - DNA, 28 bp, Artificial Sequence
  Description of Artificial Sequence Synthetic oligonucleotide
gtgttatcaactcaccagaattaagcaa

SEQ ID NO: 540 - DNA, 27 bp, Artificial Sequence
  Description of Artificial Sequence Synthetic oligonucleotide
catctcggttccgcatgatgaatctgc

SEQ ID NO: 541 - DNA, 23 bp, Artificial Sequence
  Description of Artificial Sequence Synthetic oligonucleotide
ggtagagatcttcgacatgctgc

SEQ ID NO: 542 - DNA, 26 bp, Artificial Sequence
  Description of Artificial Sequence Synthetic oligonucleotide
gttatcaactcaccagaattaagcaa

SEQ ID NO: 543 - DNA, 23 bp, Artificial Sequence
  Description of Artificial Sequence Synthetic oligonucleotide
ggtagagatcttcgacatgctgc

SEQ ID NO: 544 - DNA, 28 bp, Artificial Sequence
  Description of Artificial Sequence Synthetic oligonucleotide
tcaccagaattaagcaaaataatagatt

SEQ ID NO: 545 - DNA, 28 bp, Artificial Sequence
  Description of Artificial Sequence Synthetic oligonucleotide
catctcggttccgcatgatgaatctgca

SEQ ID NO: 546 - DNA, 24 bp, Artificial Sequence
  Description of Artificial Sequence Synthetic oligonucleotide
attcaggagtgtacacatttctgc

SEQ ID NO: 547 - DNA, 25 bp, Artificial Sequence
  Description of Artificial Sequence Synthetic oligonucleotide
cattcaggagtgtacacatttctgt

SEQ ID NO: 548 - DNA, 23 bp, Artificial Sequence
  Description of Artificial Sequence Synthetic oligonucleotide
atcaggtggatcaaagtgtctgt

SEQ ID NO: 549 - DNA, 28 bp, Artificial Sequence
  Description of Artificial Sequence Synthetic oligonucleotide
tctctggaagagaaggaccatatccacc

SEQ ID NO: 550 - DNA, 19 bp, Artificial Sequence
  Description of Artificial Sequence Synthetic oligonucleotide
acgtggtgcccctctatgg

SEQ ID NO: 551 - DNA, 18 bp, Artificial Sequence
  Description of Artificial Sequence Synthetic oligonucleotide
caagaacgtggtgcccca

SEQ ID NO: 552 - DNA, 20 bp, Artificial Sequence
  Description of Artificial Sequence Synthetic oligonucleotide
tgcaagaacgtggtgcccca

SEQ ID NO: 553 - DNA, 21 bp, Artificial Sequence
  Description of Artificial Sequence Synthetic oligonucleotide
gtgcaagaacgtggtgcccca

SEQ ID NO: 554 - DNA, 21 bp, Artificial Sequence
  Description of Artificial Sequence Synthetic oligonucleotide
gtgcaagaacgtggtgccaca

SEQ ID NO: 555 - DNA, 18 bp, Artificial Sequence
  Description of Artificial Sequence Synthetic oligonucleotide
caagaacgtggtgccccc

SEQ ID NO: 556 - DNA, 19 bp, Artificial Sequence
  Description of Artificial Sequence Synthetic oligonucleotide
caagaacgtggtgccccag

SEQ ID NO: 557 - DNA, 18 bp, Artificial Sequence
  Description of Artificial Sequence Synthetic oligonucleotide
caagaacgtggtgccccg

SEQ ID NO: 558 - DNA, 22 bp, Artificial Sequence
  Description of Artificial Sequence Synthetic oligonucleotide
tgaagtgcaagaacgtggtgca

SEQ ID NO: 559 - DNA, 24 bp, Artificial Sequence
  Description of Artificial Sequence Synthetic oligonucleotide
catgaagtgcaagaacgtggtgca

SEQ ID NO: 560 - DNA, 22 bp, Artificial Sequence
  Description of Artificial Sequence Synthetic oligonucleotide
tgaagtacaagaacgtggtgca

SEQ ID NO: 561 - DNA, 23 bp, Artificial Sequence
  Description of Artificial Sequence Synthetic oligonucleotide
atgaagtacaagaacgtggtgca

SEQ ID NO: 562 - DNA, 24 bp, Artificial Sequence
  Description of Artificial Sequence Synthetic oligonucleotide
catgaagtacaagaacgtggtgca

SEQ ID NO: 563 - DNA, 22 bp, Artificial Sequence
  Description of Artificial Sequence Synthetic oligonucleotide
tgaagtgcaagaacgtgatgca

SEQ ID NO: 564 - DNA, 26 bp, Artificial Sequence
  Description of Artificial Sequence Synthetic oligonucleotide
agcatgaagtacaagaacgtggtgca

SEQ ID NO: 565 - DNA, 22 bp, Artificial Sequence
  Description of Artificial Sequence Synthetic oligonucleotide
tgaagtgcaagaacgtggtaca

SEQ ID NO: 566 - DNA, 23 bp, Artificial Sequence
  Description of Artificial Sequence Synthetic oligonucleotide
atgaagtgcaagaacgtggtaca

SEQ ID NO: 567 - DNA, 24 bp, Artificial Sequence
  Description of Artificial Sequence Synthetic oligonucleotide
catgaagtgcaagaacgtggtaca

SEQ ID NO: 568 - DNA, 21 bp, Artificial Sequence
  Description of Artificial Sequence Synthetic oligonucleotide
catgaagtgcaagaacgtgga

SEQ ID NO: 569 - DNA, 22 bp, Artificial Sequence
  Description of Artificial Sequence Synthetic oligonucleotide
gcatgaagtgcaagaacgtgga

SEQ ID NO: 570 - DNA, 23 bp, Artificial Sequence
  Description of Artificial Sequence Synthetic oligonucleotide
agcatgaagtgcaagaacgtgga

SEQ ID NO: 571 - DNA, 20 bp, Artificial Sequence
  Description of Artificial Sequence Synthetic oligonucleotide
acgtggtgcccctctatgac

SEQ ID NO: 572 - DNA, 19 bp, Artificial Sequence
  Description of Artificial Sequence Synthetic oligonucleotide
gctttggtccgtctcctcc

SEQ ID NO: 573 - DNA, 25 bp, Artificial Sequence
  Description of Artificial Sequence Synthetic oligonucleotide
ctgctggagatgctggacgcccacc

SEQ ID NO: 574 - DNA, 19 bp, Artificial Sequence
  Description of Artificial Sequence Synthetic oligonucleotide
agaacgtggtgcccctctg

SEQ ID NO: 575 - DNA, 18 bp, Artificial Sequence
  Description of Artificial Sequence Synthetic oligonucleotide
agaacgtggtgcccctca

SEQ ID NO: 576 - DNA, 19 bp, Artificial Sequence
  Description of Artificial Sequence Synthetic oligonucleotide
aagaacgtggtgcccctca

SEQ ID NO: 577 - DNA, 20 bp, Artificial Sequence
  Description of Artificial Sequence Synthetic oligonucleotide
caagaacgtggtgcccctca

SEQ ID NO: 578 - DNA, 19 bp, Artificial Sequence
  Description of Artificial Sequence Synthetic oligonucleotide
agaacgtggtgcccctctc

SEQ ID NO: 579 - DNA, 23 bp, Artificial Sequence
  Description of Artificial Sequence Synthetic oligonucleotide
ctcctagacctcatcctctttga

(19) United States
(12) Patent Application Publication
(10) Pub. No.: US 2018/0051325 A1
(43) Pub. Date: Feb. 22, 2018

(54) MULTIPLEX ALLELE SPECIFIC PCR ASSAYS FOR DETECTION OF ESTROGEN RECEPTOR ESR1 MUTATIONS

(71) Applicant: Roche Molecular Systems, Inc., Pleasanton, CA (US)

(72) Inventors: Claudia Litterst, Walnut Creek, CA (US); Sean Chien, Danville, CA (US); Xiaoying Chen, Danville, CA (US); Ha Tran, Daly City, CA (US)

(21) Appl. No.: 15/681,039

(22) Filed: Aug. 18, 2017

Related U.S. Application Data
(60) Provisional application No. 62/376,799, filed on Aug. 18, 2016.

(51) Int. Cl. C12Q 1/68; C07K 14/72

(52) U.S. Cl. CPC C12Q 1/6827; C12N 15/00; C12Q 1/686; C07K 14/721

(57) ABSTRACT

Provided herein are methods and compositions to detect mutations in estrogen receptor (ESR1).

### CROSS-REFERENCE TO RELATED APPLICATION

The present application claims priority to U.S. Provisional Application 62/376,799, filed 18 Aug. 2016, the disclosure of which is incorporated herein in its entirety.

### SEQUENCE LISTING

The present application contains a Sequence Listing which has been submitted electronically in ASCII format and is hereby incorporated by reference in its entirety. The ASCII copy, created on Jul. 10, 2017, is named 33815-US1_SL.txt and is 137,578 bytes in size.

### BACKGROUND OF THE INVENTION

Seventy percent of breast cancers are estrogen receptor (ER) positive and, while the majority of these patients initially respond to hormone therapy, approximately 20-30% will become therapy refractory. Recent data suggest activating mutations in the estrogen receptor gene (ESR1) which are acquired during anti-estrogen treatment and rarely found in primary untreated ER positive breast cancer are associated with resistance. A growing number of activating ESR1 mutations located in the ligand-binding domain have been identified in samples from hormone-refractory breast cancer, the most common mutations include K303R, E380Q, V392I, S463P, K531E, V534E, P535H, L536Q/R, Y537S/N/C, D538G and R555C. While the mechanism of action is not fully elucidated for all mutations, it has been shown by in vitro experiments that mutations in L536Q, Y537S/C/N and D538G stabilize the estrogen receptor ligand binding domain in an active conformation, thus allowing recruitment of transcriptional coactivators in the absence of ligand. Hypersensitivity to estrogen is considered the mechanism for the K303R mutation.

Detection of ESR1 mutations thus has potential for predicting hormone resistance and directing therapy. Next generation sequencing (NGS) is a common approach for such detection, since it enables the simultaneous detection of many mutations with small amounts of samples. However, NGS is very labor-intensive, lengthy and expensive. Digital PCR (dPCR) represents a highly sensitive method that has been employed for ESR1 mutation detection, but it has a number of drawbacks: the dPCR workflow is lengthy and requires special equipment, and multiplexing is limited by the number of optical channels available on current dPCR instruments.

### SUMMARY OF THE INVENTION

Provided herein are kits, assays, and methods for detecting mutations in the ESR1 gene, and methods of treatment for individuals with hormone sensitive cancers.

Provided herein are kits comprising one or more vessels, each of the vessels holding two or more primer pairs, each primer pair specific for a different sequence in the ESR1 gene; one or more probes specific for a different sequence in the ESR1 gene, wherein each probe is labeled; a primer pair specific for an internal control sequence; and a probe specific for the internal control sequence. In some embodiments, the each probe is labeled with a fluorophore and quencher. In some embodiments, the kit comprises 1-10 vessels, e.g., 2-5, 2, 3, 4, 5, 6, 7, 8, 9, 10, or more vessels. In some embodiments, each of the vessels holds two or more primer pairs (including the internal control primer pair), e.g., 3-16, 4-10, or 5-8. For example, all exon 8 mutations, internal control, and two or more other mutation primer pairs can be included in a single tube. In some embodiments, each of the vessels holds three to four probes (including the internal control probe). In some embodiments, each vessel further holds a thermostable DNA polymerase.

In some embodiments, the kit further includes positive controls, e.g., samples with known ESR1 mutations, and/or a negative control, e.g., a sample that does not include ESR1 mutations.

In some embodiments, each different sequence of the ESR1 gene is selected from the group consisting of comprising ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H. In some embodiments the kit includes at least two primer pairs selected from the group consisting of a primer pair specific for ESR1 V422DelV, a primer pair specific for ESR1 S463P, a primer pair specific for ESR1 L536H, a primer pair specific for ESR1 L536P, a primer pair specific for ESR1 L536Q, a primer pair specific for ESR1 L536R, a primer pair specific for ESR1 D538G, ESR1 K303R, a primer pair specific for ESR1 E380Q, a primer pair specific for ESR1 L536_D538>P, a primer pair specific for ESR1 Y537C, a primer pair specific for ESR1 Y537N, a primer pair specific for ESR1 Y537S, a primer pair specific for ESR1 S341L, a primer pair specific for ESR1 L429V, a primer pair specific for ESR1 V533M, a primer pair specific for ESR1 V534E, and a primer pair specific for ESR1 P535H. In some embodiments, at least one primer includes a modified, non-naturally occurring nucleotide.

In some embodiments, the kit comprises a primer pair specific for ESR1 V422DelV, a primer pair specific for ESR1 S463P, a primer pair specific for ESR1 L536H, a primer pair specific for ESR1 L536P, a primer pair specific for ESR1 L536Q, a primer pair specific for ESR1 L536R, a primer pair specific for ESR1 D538G, ESR1 K303R, a primer pair specific for ESR1 E380Q, a primer pair specific for ESR1 L536_D538>P, a primer pair specific for ESR1 Y537C, a primer pair specific for ESR1 Y537N, a primer pair specific for ESR1 Y537S, a primer pair specific for ESR1 S341L, a primer pair specific for ESR1 L429V, a primer pair specific for ESR1 V533M, a primer pair specific for ESR1 V534E, and a primer pair specific for ESR1 P535H. In some embodiments, the kit further comprises a probe that specifically detects ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H.

In some embodiments, the kit comprises a (i) a first vessel holding a primer pair specific for ESR1 V422DelV, a primer pair specific for ESR1 S463P, a primer pair specific for ESR1 L536H, a primer pair specific for ESR1 L536P, a primer pair specific for ESR1 L536Q, a primer pair specific for L536R, and a primer pair specific for ESR1 D538G; (ii) a second vessel holding a primer pair specific for ESR1 K303R, a primer pair specific for ESR1 E380Q, a primer pair specific for ESR1 L536_D538>P, a primer pair specific for ESR1 Y537C, a primer pair specific for ESR1 Y537N, and a primer pair specific for ESR1 Y537S; and (iii) a third vessel holding a primer pair specific for ESR1 S341L, a primer pair specific for ESR1 L429V, a primer pair specific for ESR1 V533M, a primer pair specific for ESR1 V534E, and a primer pair specific for ESR1 P535H. In some embodiments, the first vessel further holds probes specific for ESR1 V422DelV, ESR1 S463P, ESR1 L536H, ESR1 L536P, ESR1 L536Q, ESR1 L536R, and ESR1 D538G. In some embodiments, a single probe specifically detects ESR1 L536H, ESR1 L536P, ESR1 L536Q, ESR1 L536R, and ESR1 D538G. In some embodiments, the second vessel further holds probes specific for ESR1 K303R, ESR1 E380Q, ESR1 L536_D538>P, ESR1 ESR1 Y537C, ESR Y537N, ESR1 Y537S. In some embodiments, a single probe specifically detects ESR1 L536_D538>P, ESR1 ESR1 Y537C, ESR Y537N, ESR1 Y537S. In some embodiments, the third vessel holds probes specific for ESR1 S341L, ESR1 L429V, ESR1 V533M, ESR1 V534E, and ESR1 P535H. In some embodiments, a single probe specifically detects ESR1 V533M, ESR1 V534E, and ESR1 P535H.

In some embodiments, the allele-specific primer to detect the ESR1 K303R mutation is selected from the group consisting of SEQ ID NOs: 479, 481, and 484, and in some embodiments, the common primer and probe sequences are SEQ ID NOs:476 and 477, respectively. In some embodiments, the allele-specific primer to detect the ESR1 S341L mutation is selected from the group consisting of SEQ ID NOs:239, 240, 242, 245, 246, 247, 253, 254, 255, 256, 257, 258, and 259, and in some embodiments, the common primer and probe sequences are SEQ ID NOs:235 and 237, respectively. In some embodiments, the allele-specific primer to detect the ESR1 E380Q mutation is selected from the group consisting of SEQ ID NOs:32, 34, 36, 37, 38, 39, 40, 41, and 42, and in some embodiments, the common primer and probe sequences are SEQ ID NOs:528 and 31, respectively. In some embodiments, the allele-specific primer to detect the ESR1 V422DELV mutation is selected from the group consisting of SEQ ID NOs:287, 294, 295, 296, 297, 298, and in some embodiments, the common primer and probe sequences are SEQ ID NOs:285 and 286, respectively. In some embodiments, the allele-specific primer to detect the ESR1 L429V mutation is selected from the group consisting of SEQ ID NOs:66, 68, 69, 70, 72, 73, 75, 76, 77, 778, 79, 80, and 81, and in some embodiments, the common primer and probe sequences are SEQ ID NOs:59 and 60, respectively. In some embodiments, the allele-specific primer to detect the ESR1 S463P mutation is selected from the group consisting of SEQ ID NOs:264, 265, 267, 268, 269, 270, 271, 272, 281, 282, 283, and 275, and in some embodiments, the common primer and probe sequences are SEQ ID NOs:261 and 262, respectively.

In some embodiments, a common primer and probe are used to amplify and detect all ESR1 exon 8 mutations in the assay, e.g., SEQ ID NOs:314 and/ or 394, and 407, respectively. In some embodiments, the allele-specific primer to detect the ESR1 V533M mutation is selected from the group consisting of SEQ ID NOs:322, 329, 330, 345, 346, 347, 348, 349, 350, 352, and 353. In some embodiments, the allele-specific primer to detect the ESR1 V534E mutation is selected from the group consisting of SEQ ID NOs:364, 365, 367, 368, 370, 371, 373, 378, 388, 391, 392, and 393. In some embodiments, the allele-specific primer to detect the ESR1 P535H mutation is selected from the group consisting of SEQ ID NOs:181, 188, 189, 190, 191, 198, 199, 200, 203, 204, 205, 208, 209, 212, 225, 226, 228, 229, and 231. In some embodiments, the allele-specific primer to detect the ESR1 L536H mutation is selected from the group consisting of SEQ ID NOs:96, 101, 104, 105, 107, 110, 111, 112, 113, 114, 115, 116, and 117. In some embodiments, the allele-specific primer to detect the ESR1 L536P mutation is selected from the group consisting of SEQ ID NOs:134, 135, and 137. In some embodiments, the allele-specific primer to detect the ESR1 L536Q mutation is selected from the group consisting of SEQ ID NOs:141, 151, 154, 155, 157, 158, 159, and 160. In some embodiments, the allele-specific primer to detect the ESR1 L536R mutation is selected from the group consisting of SEQ ID NOs:161, 172, 174, 175, 176, 177, 178, 179, and 180. In some embodiments, the allele-specific primer to detect the ESR1 Y537C mutation is selected from the group consisting of SEQ ID NOs:397, 408, 415, 416, 417, 418, 419, 420, and 424. In some embodiments, the allele-specific primer to detect the ESR1 Y537N mutation is selected from the group consisting of SEQ ID NOs:426, 436, 441, 445, 446, 447, and 448. In some embodiments, the allele-specific primer to detect the ESR1 Y537S mutation is selected from the group consisting of SEQ ID NOs:449, 459, 466, 467, 468, 469, 470, 471, and 472. In some embodiments, the allele-specific primer to detect the ESR1 D538G mutation is selected from the group consisting of SEQ ID NOs: 14, 21, 22, 23, 24, and 25. In some embodiments, the allele-specific primer to detect the ESR1 L536_D538>P mutation is selected from the group consisting of SEQ ID NOs:84, 85, 86, 87, 88, 89, 90, 91, 92, and 93.

In some embodiments, the kit includes oligonucleotides having the sequences of SEQ ID NOs:24, 113, 134, 158, 178, 264, 261, 262, 285, 286, 298, 314, 394, 407, 31, 42, 90, 415, 447, 469, 476, 477, 481, 528, 59, 60, 70, 231, 235, 237, 245, 350, and 388. In some embodiments, each vessel includes an internal control, e.g., SEQ ID NOs:511, 514, and 517.

Further provided are methods for determining (e.g., detecting) the presence or absence of two or more ESR1 mutations in a sample from an individual, comprising (i) obtaining a sample from the individual; (ii) carrying out multiplex allele-specific PCR to determine the presence or absence of two or more ESR1 mutations. In some embodiments, the sample is selected from blood, plasma, serum, urine, or mucosal tissue (e.g., from a buccal swab). In some embodiments, the individual has, or was diagnosed with, hormone responsive cancer (e.g., estrogen receptor and/or progesterone receptor positive breast or ovarian cancer). In some embodiments, the individual is undergoing hormone therapy. In some embodiments, the method further comprises providing hormone therapy to the individual prior to step (i) (e.g., treatment with a SERM, aromatase inhibitor, or LH blocking agent). In some embodiments, the method further comprises providing modified treatment to the individual when the presence of an ESR1 mutation is determined in step (ii) (e.g., an additional hormone therapy, or standard chemotherapy).

In some embodiments, the method comprises carrying out multiplex allele-specific PCR to determine the presence or absence of ten or more ESR1 mutations, e.g., 10-20, 11, 12, 13, 14, 15, 16, 17, 18, or 19. In some embodiments, the two or more ESR1 mutations are selected from ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H. In some embodiments, the two or more ESR1 mutations include ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H.

In some embodiments, the multiplex allele-specific PCR is carried out using a kit as described herein, e.g., comprising 1-10 vessels, e.g., 2-5, 2, 3, 4, 5, 6, 7, 8, 9, 10, or more vessels. In some embodiments, the kit comprises three vessels. For example, the allele-specific multiplex PCR can be carried out in three vessels such that (i) ESR1 V422DelV, ESR1 S463P, ESR1 L536H, ESR1 L536P, ESR1 L536Q, L536R, and ESR1 D538G are detected in a first vessel; (ii) ESR1 K303R, ESR1 E380Q, ESR1 L536_D538>P, ESR1 Y537C, ESR1 Y537N, and ESR1 Y537S are detected in a second vessel; and (iii) ESR1 S341L, ESR1 L429V, ESR1 V533M, ESR1 V534E, and ESR1 P535H are detected in a third vessel.

Further provided herein are methods of providing modified treatment of an individual with a hormone responsive cancer (e.g., breast cancer) that is undergoing hormone therapy (e.g., SERM, aromatase inhibitors, and/or lutenizing hormone blockers). In some embodiments, the method comprises (i) obtaining a sample from the individual (e.g., blood, plasma, serum, urine, tissue, FFPET, etc.); (ii) carrying out multiplex allele-specific PCR to determine the presence or absence of two or more ESR1 mutations; and (iii) providing modified treatment of the individual if the presence of an ESR1 mutation is determined. In some embodiments, step (ii) is carried out using a kit as described herein.

In some embodiments, the multiplex allele-specific PCR determines the presence or absence of ten or more ESR1 mutations. In some embodiments, the two or more (or ten or more) ESR1 mutations are selected from the group consisting of ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H. In some embodiments, the presence or absence of ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H is determined.

In some embodiments, steps (i)-(iii) are carried out 0.5 to 5 years after the individual starts taking hormone therapy. In some embodiments, the method is carried out more than once during hormone therapy, e.g., to monitor potential resistance of the tumor to hormone therapy. In some embodiments, the method is carried out periodically, e.g., every 6 months, while in some embodiments, the method is carried out upon clinical progression in the individual (e.g., tumor growth, reduced response to hormone therapy, etc.). In some embodiments, step (iii) comprises providing an additional hormone therapy or standard chemotherapy to the individual.

In addition, provided herein are methods of treating an individual with a hormone responsive cancer (e.g., breast or ovarian cancer). In some embodiments, the method comprises (i) providing hormone therapy to the individual; (ii) obtaining a sample from the individual; (iii) carrying out multiplex allele-specific PCR to determine the presence or absence of two or more ESR1 mutations; and (iv) providing modified treatment to the individual if the presence of an ESR1 mutation is determined. In some embodiments, the hormone therapy is SERM.

In some embodiments, the multiplex allele-specific PCR determines the presence or absence of ten or more ESR1 mutations. In some embodiments, the two or more (or ten or more) ESR1 mutations are selected from the group consisting of ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H. In some embodiments, the presence or absence of ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H is determined.

In some embodiments, steps (ii)-(iii) are carried out 0.5-5 years after step (i) (after the individual starts taking hormone therapy). In some embodiments, steps (ii)-(iii) are carried out more than once during hormone therapy. In some embodiments, the method is carried out periodically, e.g., every 6 months, while in some embodiments, the method is carried out upon clinical progression in the individual (e.g., tumor growth, reduced response to hormone therapy, etc.). In some embodiments, step (iv) comprises providing an additional hormone therapy (e.g., aromatase inhibitor and/or lutenizing hormone blocker) or standard chemotherapy to the individual.

### BRIEF DESCRIPTION OF THE DRAWINGS

FIG. 1A, FIG. 1B, and FIG. 1C compare discrimination of primers and probes for detecting mutant and wild type samples. FIG. 1A shows results (amplification growth curves) using primers and a probe designed to be specific for K303R that shows poor discrimination between wild type and mutant. FIG. 1B shows results using primers and a probe designed to be specific for K303R that shows better discrimination between wild type and mutant. FIG. 1C shows results using primers and a probe designed to be specific for L536_D538>P that shows good specificity detecting mutant compared to wild type sample, with wild type sample showing no signal.

FIG. 2A, FIG. 2B, and FIG. 2C also compare discrimination of primers and probes for detecting the L536R mutant and wild type samples. The amplification growth curves show cycles on the X axis and signal on the Y axis. FIG. 2A shows results using the L536R_RS01 allele-specific primer that shows poor discrimination between wild type and mutant. FIG. 2B shows results using the L536R_RS09 allele-specific primer that shows better discrimination between wild type and mutant. FIG. 2C shows results using the L536R_RS04 allele-specific primer that shows good specificity detecting mutant compared to wild type sample, with wild type sample showing essentially no signal.

FIG. 3 shows specificity of primers and probe designed to be specific for the P535H mutation compared to other ESR1 mutant samples.

FIG. 4A shows amplification growth curves for an E380Q mutation positive control (MC, top/left two curves), a E380Q mutation positive sample (IN008, next lower two curves), E380 wild type (FFPET_WT, next lower two curves), and non-template control (NTC, flat bottom line).

FIG. 4B shows the same amplification growth curves without the E380Q mutation positive control curves.

FIG. 5 shows amplification growth curves for a D538G mutation positive control (MC, top/left curve), 3 D538G mutation positive samples (D538G, next lower three curves), D538 wild type (next lower 48 curves), and non-template control (NTC, flat bottom line).

### DETAILED DESCRIPTION OF THE INVENTION

### I. INTRODUCTION

Provided herein is a multiplex PCR assay for detecting the presence of mutations in the human estrogen receptor (ESR1). These mutations are associated with resistance to hormone therapy in cancer patients with hormone responsive cancers (ESR1 positive and/or progesterone receptor positive) that were sensitive to hormone therapy when initially administered. The assays can be carried out with non-invasive samples (e.g., blood, plasma, serum, urine, etc.) so that repeated testing can be carried out in a patient on hormone therapy without taking multiple tissue biopsies.

The presently described assays rely on proven, widely adopted technology and provide accurate, reproducible, and rapid results.

### II. DEFINITIONS

The terms “estrogen receptor”, “ER”, and “ESR1” are used interchangeably herein unless otherwise noted. ESR1 can also be used to refer to the gene encoding the ER protein.

The term “multiplex” refers to an assay in which more than one target is detected. The terms “receptacle,” “vessel,” “tube,” “well,” “chamber,” “microchamber,” etc. refer to a container that can hold reagents or an assay. If the receptacle is in a kit and holds reagents, or is being used for an amplification reaction, it can be closed or sealed to avoid contamination or evaporation. If the receptacle is being used for an assay, it can be open or accessible, at least during set up of the assay.

The terms “individually detected” or “individual detection,” referring to a marker gene or marker gene product, indicates that each marker in a multiplex reaction is detected. That is, each marker is associated with a different label (detected by a differently labeled probe).

The terms “nucleic acid,” “polynucleotide,” and “oligonucleotide” refer to polymers of nudeotides (e.g., ribonucleotides or deoxyribo-nudeotides) and includes naturally-occurring (adenosine, guanidine, cytosine, uracil and thymidine), non-naturally occurring, and modified nucleic acids. The term is not limited by length (e.g., number of monomers) of the polymer. A nucleic acid may be single-stranded or double-stranded and will generally contain 5′-3′ phosphodiester bonds, although in some cases, nucleotide analogs may have other linkages. Monomers are typically referred to as nucleotides. The term “non-natural nucleotide” or “modified nucleotide” refers to a nucleotide that contains a modified nitrogenous base, sugar or phosphate group, or that incorporates a non-natural moiety in its structure. Examples of non-natural nucleotides include dideoxynucleotides, biotinylated, aminated, deaminated, alkylated, benzylated and fluorophor-labeled nudeotides.

The term “primer” refers to a short nucleic acid (an oligonucleotide) that acts as a point of initiation of polynucleotide strand synthesis by a nucleic acid polymerase under suitable conditions. Polynucleotide synthesis and amplification reactions typically include an appropriate buffer, dNTPs and/or rNTPs, and one or more optional cofactors, and are carried out at a suitable temperature. A primer typically includes at least one target-hybridized region that is at least substantially complementary to the target sequence (e.g., having 0, 1, 2, or 3 mismatches). This region of is typically about 8 to about 40 nucleotides in length, e.g., 12-25 nucleotides. A “primer pair” refers to a forward and reverse primer that are oriented in opposite directions relative to the target sequence, and that produce an amplification product in amplification conditions. In some embodiments, multiple primer pairs rely on a single common forward or reverse primer. For example, multiple allele-specific forward primers can be considered part of a primer pair with the same, common reverse primer, e.g., if the multiple alleles are in close proximity to each other.

As used herein, “probe” means any molecule that is capable of selectively binding to a specifically intended target biomolecule, for example, a nucleic acid sequence of interest that hybridizes to the probes. The probe is detectably labeled with at least one non-nucleotide moiety. In some embodiments, the probe is labeled with a fluorophore and quencher.

The words “complementary” or “complementarity” refer to the ability of a nucleic acid in a polynucleotide to form a base pair with another nucleic acid in a second polynucleotide. For example, the sequence A-G-T (A-G-U for RNA) is complementary to the sequence T-C-A (U-C-A for RNA). Complementarity may be partial, in which only some of the nucleic acids match according to base pairing, or complete, where all the nucleic acids match according to base pairing. A probe or primer is considered “specific for” a target sequence if it is at least partially complementary to the target sequence. Depending on the conditions, the degree of complementarity to the target sequence is typically higher for a shorter nucleic acid such as a primer (e.g., greater than 80%, 90%, 95%, or higher) than for a longer sequence. In some embodiments, the term “each primer pair specific for a different sequence in the ESR1 gene” indicates that each primer pair specifically amplifies a different sequence, e.g., a different allele or mutation, of the ESR1 gene.

The term “specifically amplifies” indicates that a primer set amplifies a target sequence more than non-target sequence at a statistically significant level. The term “specifically detects” indicates that a probe will detect a target sequence more than non-target sequence at a statistically significant level. As will be understood in the art, specific amplification and detection can be determined using a negative control, e.g., a sample that includes the same nucleic acids as the test sample, but not the target sequence or a sample lacking nucleic acids. For example, primers and probes that specifically amplify and detect a target sequence result in a Ct that is readily distinguishable from background (non-target sequence), e.g., a Ct that is at least 2, 3, 4, 5, 5-10, 10-20, or 10-30 cycles less than background. The term “allele-specific” PCR refers to amplification of a target sequence using primers that specifically amplify a particular allelic variant of the target sequence. Typically, the forward or reverse primer includes the exact complement of the allelic variant at that position.

The terms “identical” or “percent identity,” in the context of two or more nucleic acids, or two or more polypeptides, refer to two or more sequences or subsequences that are the same or have a specified percentage of nucleotides, or amino acids, that are the same (e.g., about 60% identity, e.g., at least any of 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters, or by manual alignment and visual inspection. See e.g., the NCBI web site at ncbi.nlm.nih.gov/BLAST. Such sequences are then said to be “substantially identical.” Percent identity is typically determined over optimally aligned sequences, so that the definition applies to sequences that have deletions and/or additions, as well as those that have substitutions. The algorithms commonly used in the art account for gaps and the like. Typically, identity exists over a region comprising an a sequence that is at least about 8-25 amino acids or nucleotides in length, or over a region that is 50-100 amino acids or nucleotides in length, or over the entire length of the reference sequence.

The term “kit” refers to any manufacture (e.g., a package or a container) including at least one reagent, such as a nucleic acid probe or probe pool or the like, for specifically amplifying, capturing, tagging/converting or detecting RNA or DNA as described herein.

The term “amplification conditions” refers to conditions in a nucleic acid amplification reaction (e.g., PCR amplification) that allow for hybridization and template-dependent extension of the primers. The term “amplicon” or “amplification product” refers to a nucleic acid molecule that contains all or a fragment of the target nucleic acid sequence and that is formed as the product of in vitro amplification by any suitable amplification method. One of skill will understand that a forward and reverse primer (primer pair) defines the borders of an amplification product. The term “generate an amplification product” when applied to primers, indicates that the primers, under appropriate conditions (e.g., in the presence of a nucleotide polymerase and NTPs), will produce the defined amplification product. Various PCR conditions are described in PCR Strategies (Innis et al., 1995, Academic Press, San Diego, Calif.) at Chapter 14; PCR Protocols: A Guide to Methods and Applications (Innis et al., Academic Press, NY, 1990)

The term “amplification product” refers to the product of an amplification reaction. The amplification product includes the primers used to initiate each round of polynucleotide synthesis. An “amplicon” is the sequence targeted for amplification, and the term can also be used to refer to amplification product. The 5′ and 3′ borders of the amplicon are defined by the forward and reverse primers.

The terms “individual”, “subject”, and “patient” are used interchangeably herein. The individual can be pre-diagnosis, post-diagnosis but pre-therapy, undergoing therapy, or post-therapy. In the context of the present disclosure, the individual is typically seeking medical care.

The term “sample” or “biological sample” refers to any composition containing or presumed to contain nucleic acid. The term includes purified or separated components of cells, tissues, or blood, e.g., DNA, RNA, proteins, cell-free portions, or cell lysates. The sample can be FFPET, e.g., from a tumor or metastatic lesion. The sample can also be from frozen or fresh tissue, or from a liquid sample, e.g., blood or a blood component (plasma or serum), urine, semen, saliva, sputum, mucus, semen, tear, lymph, cerebral spinal fluid, mouth/throat rinse, bronchial alveolar lavage, material washed from a swab, etc. Samples also may include constituents and components of in vitro cultures of cells obtained from an individual, including cell lines. The sample can also be partially processed from a sample directly obtained from an individual, e.g., cell lysate or blood depleted of red blood cells.

The term “obtaining a sample from an individual” means that a biological sample from the individual is provided for testing. The obtaining can be directly from the individual, or from a third party that directly obtained the sample from the individual.

The term “providing therapy for an individual” means that the therapy is prescribed, recommended, or made available to the individual. The therapy may be actually administered to the individual by a third party (e.g., an in-patient injection), or by the individual herself.

A “control” sample or value refers to a value that serves as a reference, usually a known reference, for comparison to a test sample or test conditions. For example, a test sample can be taken from a test condition, e.g., from an individual suspected of having cancer, and compared to samples from known conditions, e.g., from a cancer-free individual (negative control), or from an individual known to have cancer (positive control). In the context of the present disclosure, the test sample is typically from a breast cancer patient. A control can also represent an average value or a range gathered from a number of tests or results. A control can also be prepared for reaction conditions. For example, a control for the presence, quality, and/ or quantity of nucleic acid (e.g., internal control) can include primers or probes that will detect a sequence known to be present in the sample (e.g., a housekeeping gene such as beta actin, beta globin, glyceraldehyde 3-phosphate dehydrogenase (GAPDH), ribosomal protein L37 and L38, PPlase, EIF3, eukaryotic translation elongation factor 2 (eEF2), DHFR, or succinate dehydrogenase). In some embodiments, the internal control can be a sequence from a region of the same gene that is not commonly variant (e.g., in a different exon). A known added polynucleotide, e.g., having a designated length, can also be added. An example of a negative control is one free of nucleic acids, or one including primers or probes specific for a sequence that would not be present in the sample, e.g., from a different species. One of skill will understand that the selection of controls will depend on the particular assay, e.g., so that the control is cell type and organism-appropriate. One of skill in the art will recognize that controls can be designed for assessment of any number of parameters. For example, a control can be devised to compare therapeutic benefit based on pharmacological data (e.g., half-life) or therapeutic measures (e.g., comparison of benefit and/or side effects). Controls can be designed for in vitro applications. One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The terms “label,” “tag,” “detectable moiety,” and like terms refer to a composition detectable by spectroscopic, photochemical, biochemical, immunochemical, chemical, or other physical means. For example, useful labels include fluorescent dyes (fluorophores), luminescent agents, radioisotopes (e.g., ³²P, ³H), electron-dense reagents, or an affinity-based moiety, e.g., a poly-A (interacts with poly-T) or poly-T tag (interacts with poly-A), a His tag (interacts with Ni), or a strepavidin tag (separable with biotin). One of skill will understand that a detectable label conjugated to a nucleic acid is not naturally occurring.

Unless defined otherwise, technical and scientific terms used herein have the same meaning as commonly understood by a person of ordinary skill in the art. See, e.g., Lackie, DICTIONARY OF CELL AND MOLECULAR BIOLOGY, Elsevier (4th ed. 2007); Sambrook et al., MOLECULAR CLONING, A LABORATORY MANUAL, Cold Springs Harbor Press (Cold Springs Harbor, N.Y. 1989). The term “a” or “an” is intended to mean “one or more.” The terms “comprise,” “comprises,” and “comprising,” when preceding the recitation of a step or an element, are intended to mean that the addition of further steps or elements is optional and not excluded.

### III. NUCLEIC ACID SAMPLES

Samples for nucleic acid amplification can be obtained from any source suspected of containing nucleic acid. Samples can be taken from formalin fixed paraffin embedded tissue (FFPET), tissue biopsy, or cultured cells (e.g., obtained from a patient, or representing a control). In some embodiments, the sample is obtained in a non-invasive manner, e.g., from urine, skin, swab, saliva, blood or a blood fraction.

In a sample that includes cells, the cells can be separated out (e.g., using size-based filtration or centrifugation), thereby leaving cell free nucleic acids (cfNA), including nucleic acids in exosomes, microvesicles, viral particles, or those circulating freely. Alternatively, the cells can be lysed to obtain cellular nucleic acids, either in the presence of magnetic glass particles (MGPs) or before addition of the cellular lysate to the MGPs.

Methods for isolating nucleic acids from biological samples are known, e.g., as described in Sambrook, and several kits are commercially available (e.g., High Pure RNA Isolation Kit, High Pure Viral Nudeic Acid Kit, and MagNA Pure LC Total Nudeic Acid Isolation Kit, DNA Isolation Kit for Cells and Tissues, DNA Isolation Kit for Mammalian Blood, High Pure FFPET DNA Isolation Kit, available from Roche). In the context of the presently disclosed methods, RNA is collected, though in some embodiments, the classifier can be used on previously prepared cDNA.

### IV. ESTROGEN RECEPTOR MUTATED CANCER AND THERAPIES

Hormone sensitive tumors are commonly treated with hormone therapy. Hormone insensitive tumors typically do not respond to hormone therapy. The term “hormone therapy” applies to a variety of treatments that block the effect of hormones that affect growth of the tumor. In some cases however, such as certain womb and kidney cancers, progesterone or a synthetic version thereof is prescribed.

Breast cancer is often hormone sensitive, and patients diagnosed with breast cancer are tested to determine if the tumor is estrogen receptor (ESR1) and/ or progesterone receptor positive. Receptor positive tumors can be treated with agents that interfere with production of these hormones. Ovarian ablation can be carried out to remove ovaries using surgery or radiation. Surgery is often followed up with additional chemotherapy. Selective estrogen receptor modulators (SERMS) that block the effect of estrogen on the ESR include tamoxifen, raloxifene, toremifene, and fulvestrant. Additional treatments include aromatase inhibitors (e.g., anastrozole, exemestane, letrozole) and lutenizing hormone (LH) blockers (e.g., goserelin, leuprolide). These therapies can be used in combination, e.g., a SERM with an aromatase inhibitor for post-menopausal patient, or a SERM with an LH blocker for pre-menopausal patient. Similar therapies are considered effective for receptor positive ovarian cancers, while prostate cancer can be treated with lutenizing hormone blockers, anti-androgens, and/ or gonadotrophin releasing hormone blockers.

In addition, patients can benefit from standard chemotherapy. This can include CHOP (cyclophosphamide; doxorubicin; vincristine; and prednisolone) or R-CHOP, which further includes rituximab and/or etoposide. The cocktail can be administered periodically for a set period of time, or until reduction in tumor size and/or symptoms are detected. For example, the CHOP or R-CHOP can be administered every 2 or 3 weeks.

Regardless of which treatment is selected, it typically begins with a low dose so that side effects can be determined, and the dose increased, e.g., until side effects appear or within the patient's tolerance, or until clinical benefit is observed.

After initial treatment, patients can become resistant to hormone therapy. Resistance to hormone therapy is thought to be due, at least in part, to mutations in the estrogen receptor. Many of these mutations are in the ligand binding domain, so that the receptor is active in the absence of estrogen release. Examples include K303R, E380Q, V392I, S463P, K531E, V534E, P535H, L536Q/R, Y537S/C/N, D538G, and R555C. Testing for mutations in the estrogen receptor allows for more effective therapeutic decisions for patients. The testing can be periodic during hormone therapy, e.g., before, during, or after observation of resistance. For example, a patient receiving SERM therapy can be switched to a different hormone therapy or combination thereof, or to standard chemotherapy.

### V. AMPLIFICATION AND DETECTION

A nucleic acid sample can be used for detection and quantification, e.g., using nucleic acid amplification, e.g., using any primer-dependent method. In some embodiments, a preliminary reverse transcription step is carried out (also referred to as RT-PCR, not to be confused with real time PCR). See, e.g., Hierro et al. (2006) 72:7148. The term “qRT-PCR” as used herein refers to reverse transcription and quantitative PCR. Both reactions can be carried out in a single tube without interruption, e.g., to add reagents. For example, a polyT primer can be used to reverse transcribe all mRNAs in a sample with a polyA tail, random oligonucleotides can be used, or a primer can be designed that is specific for a particular target transcript that will be reverse transcribed into cDNA. The cDNA, or DNA from the sample, can form the initial template to be for quantitative amplification (real time or quantitative PCR, i.e., RTPCR or qPCR). qPCR allows for reliable detection and measurement of products generated during each cycle of PCR process. Such techniques are well known in the art, and kits and reagents are commercially available, e.g., from Roche Molecular Systems, Life Technologies, Bio-Rad, etc. See, e.g., Pfaffl (2010) Methods: The ongoing evolution of qPCR vol. 50.

A separate reverse transcriptase and thermostable DNA polymerase can be used, e.g., in a two-step (reverse transcription followed by addition of DNA polymerase and amplification) or combined reaction (with both enzymes added at once). In some embodiments, the target nucleic acid is amplified with a thermostable polymerase with both reverse transcriptase activity and DNA template-dependent activity. Exemplary enzymes include Tth DNA polymerase, the C. therm Polymerase system, and those disclosed in 0520140170730 and US20140051126.

Probes for use as described herein can be labeled with a fluorophore and quencher (e.g., TaqMan, LightCycler, Molecular Beacon, Scorpion, or Dual Labeled probes). Appropriate fluorophores include FAM, JOE, TET, Cal Fluor Gold 540, HEX, VIC, Cal Fluor Orang 560, TAMRA, Cyanine 3, Quasar 570, Cal Fluor Red 590, Rox, Texas Red, Cyanine 5, Quasar 670, and Cyanine 5.5. Appropriate quenchers include TAMRA (for FAM, JOE, and TET), DABCYL, and BHQ1-3.

Detection devices are known in the art and can be selected as appropriate for the selected labels. Detection devices appropriate for quantitative PCR include the cobas® and Light Cycler® systems (Roche), PRISM 7000 and 7300 real-time PCR systems (Applied Biosystems), etc. Six-channel detection is available on the CFX96 Real Time PCR Detection System (Bio-Rad) and Rotorgene Q (Qiagen), allowing for a higher degree of multiplexing.

Results can be expressed in terms of a threshold cycle (abbreviated as Ct, and in some instances Cq or Cp). A lower Ct value reflects the rapid achievement of a predetermined threshold level, e.g., because of higher target nucleic acid concentration or a more efficient amplification. A higher Ct value may reflect lower target nucleic acid concentration, or inefficient or inhibited amplification. The threshold cycle is generally selected to be in the linear range of amplification for a given target. In some embodiments, the Ct is set as the cycle at which the growth signal exceeds a pre-defined threshold line, e.g., in relation to the baseline, or by determining the maximum of the second derivation of the growth curve. Determination of Ct is known in the art, and described, e.g., in U.S. Pat. No. 7363168.

### VI. KITS

Provided herein are kits for carrying out multiplex, allele-specific PCR to detect ESR1 mutations. The kits include primers and probes for specifically detecting particular ESR1 mutations as described herein.

A primer pair and probe for detecting ESR1 V422DelV can be selected from forward primer sequences SEQ ID NOs:284 or 287-300, reverse primer sequences SEQ ID NOs:285 or 302-311, and probe sequences SEQ ID NOs:286 or 301. A primer pair and probe for detecting ESR1 S463P can be selected from forward primer sequences SEQ ID NOs:260 or 263-272, reverse primer sequences SEQ ID NOs:261 or 274-283, and probe sequences SEQ ID NOs:262 or 273. A primer pair and probe for detecting ESR1 L536H can be selected from forward primer sequences SEQ ID NOs:96-105, reverse primer sequences SEQ ID NOs:106-120, and probe sequences SEQ ID NOs:94 or 95. A primer pair for detecting ESR1 L536P can be selected from forward primer sequences SEQ ID NOs:121-130 and reverse primer sequences SEQ ID NOs:131-140. A primer pair for detecting ESR1 L536Q can be selected from forward primer sequences SEQ ID NOs:141-150 and reverse primer sequences SEQ ID NOs:151-160. A primer pair for detecting ESR1 L536R can be selected from forward primer sequences SEQ ID NOs:161-170 and reverse primer sequences SEQ ID NOs:171-180. A primer pair for detecting ESR1 D538G can be selected from forward primer sequences SEQ ID NOs:4-13 and reverse primer sequences SEQ ID NOs:14-27 or 550. A primer pair and probe for detecting ESR1 K303R can be selected from forward primer sequences SEQ ID NOs:473, 474, or 478-487, reverse primer sequences SEQ ID NOs:477, 489-498 or 576, and probe sequences SEQ ID NOs:477 or 488. A primer pair and probe for detecting ESR1 E380Q can be selected from forward primer sequences SEQ ID NOs:28 or 32-42, reverse primer sequences SEQ ID NOs:29, 30, or 44-53, and probe sequences SEQ ID NOs:43 or 54. Primers for detecting ESR1 L536_D538>P can be selected from reverse primer sequences SEQ ID NOs:84-93. A primer pair and probe for detecting ESR1 Y537C can be selected from forward primer sequences SEQ ID NOs:394 or 397-406, reverse primer sequences SEQ ID NOs:395, 408-425, or 574, and probe sequences SEQ ID NOs:396 or 407. A primer pair for detecting ESR1 Y537N can be selected from forward primer sequences SEQ ID NOs:426-435 and reverse primer sequences SEQ ID NOs:436-448 or 575-577. A primer pair for detecting ESR1 Y537S can be selected from forward primer sequences SEQ ID NOs:449-458 and reverse primer sequences SEQ ID NOs:459-472 or 578. A primer pair and probe for detecting ESR1 S341L can be selected from forward primer sequences SEQ ID NOs:232, 233, or 238-247, reverse primer sequences SEQ ID NOs:234, 235, or 250-259, and probe sequences SEQ ID NOs:248 or 249. A primer pair and probe for detecting ESR1 L429V can be selected from forward primer sequences SEQ ID NOs:58 or 61-70, reverse primer sequences SEQ ID NOs:59 or 72-81, and probe sequences SEQ ID NOs:60 or 71. A primer pair and probe for detecting ESR1 V533M can be selected from forward primer sequences SEQ ID NOs:312-318, 322-333, 355, or 356, reverse primer sequences SEQ ID NOs:319, 320, or 336354, and probe sequences SEQ ID NOs:321, 334, or 335. A primer pair and probe for detecting ESR1 V534E can be selected from forward primer sequences SEQ ID NOs:357 o r363-373, reverse primer sequences SEQ ID NOs:358-362 or 378-393, and probe sequences SEQ ID NOs:363 or 374-377. A primer pair for detecting ESR1 P535H can be selected from forward primer sequences SEQ ID NOs:181-190 and reverse primer sequences SEQ ID NOs:191-231.

In addition, for mutations in exon 8, a common forward or common reverse primer can be used to amplify each amplification product, and a common probe can be used to detect each amplification product. One of skill will understand that slight variations can be made to the sequences, e.g., addition or deletion of 1-3 nucleotides.

Allele-specific primers selected from those disclosed in Table 5 for each mutation can be included in the kit, along with common primers and probes corresponding to that mutation.

In some embodiments, the kit comprises 1-18 stock solutions comprising allele-specific primer pairs and probes. The stock solutions can optionally include DNA polymerase, and optionally include a primer pair and probe to detect an internal control. In some embodiment, the kit includes 2-5 such stock solutions, e.g., 2, 3, 4, or 5 stock solutions.

In some embodiments, the stock solution mixtures further comprise buffers, dNTPs, and other elements (e.g., cofactors or aptamers) appropriate for reverse transcription and amplification. Typically, the mixture is concentrated, so that an aliquot is added to the final reaction volume, along with sample (e.g., DNA), enzymes, and/ or water. In some embodiments, the kit further comprises reverse transcriptase (or an enzyme with reverse transcriptase activity), and/or DNA polymerase (e.g., thermostable DNA polymerase such as Taq, ZO5, and derivatives thereof).

In some embodiments, the kit further includes components for DNA or RNA purification from a sample, e.g., a non-invasive or tissue sample. For example, the kit can include components from MagNA Pure LC Total Nucleic Acid Isolation Kit, DNA Isolation Kit for Cells and Tissues, DNA Isolation Kit for Mammalian Blood, High Pure FFPET DNA Isolation Kit, High Pure or MagNA Pure RNA Isolation Kits (Roche), DNeasy or RNeasy Kits (Qiagen), PureLink DNA or RNA Isolation Kits (Thermo Fisher), etc.

In some embodiments, the kit further includes at least one control sample, e.g., nucleic acids from non-cancer sample (or pooled samples), or from a known ESR1 mutated sample (or pooled samples). In some embodiments, the kit includes a negative control, e.g., lacking nucleic acids, or lacking mutated ESR1 nucleic acids. In some embodiments, the kit further includes consumables, e.g., plates or tubes for nucleic acid preparation, tubes for sample collection, etc. In some embodiments, the kit further includes instructions for use, reference to a website, or software.

### VII. EXAMPLES

### Assay Design

Multiplex, allele-specific PCR assays to detect 18 mutations in the ESR1 gene were designed. The mutations detected are shown in Table 1.

TABLE 1
ESR1 mutations

| | ESR1 mutation (AA) | ESR1 mutation (NA) | Exon |
|---|---|---|---|
| 1 | K303R | 908 A > G | 4 |
| 2 | S341L | 1022 C > T | 4 |
| 3 | E380Q | 1138 G > C | 5 |
| 4 | V422DelV | 1262_1264delTGG | 6 |
| 5 | L429V | 1285 C > G | 6 |
| 6 | S463P | 1387 T > C | 7 |
| 7 | V533M | 1597 G > A | 8 |
| 8 | V534E | 1601 T > A | 8 |
| 9 | P535H | 1604 C > A | 8 |
| 10 | L536Q | 1607/8 TC > AC | 8 |
| 11 | L536R | 1607 T > G | 8 |
| 12 | L536H | 1607 T > A | 8 |
| 13 | L536P | 1607 T > C | 8 |
| 14 | L536_D538 > P | 1607_1614 TCTATGAC > CC | 8 |
| 15 | Y537N | 1609 T > A | 8 |
| 16 | Y537C | 1610 A > G | 8 |
| 17 | Y537S | 1610 A > C | 8 |
| 18 | D538G | 1613 A > G | 8 |

The primers and probes generated for the assays are shown in Table 2. One of skill will understand that the list is not exhaustive, and that primers and/or probe sequences can be slightly modified from those shown, e.g., adding or subtracting one or a few nucleotides, shifting the position of the modified nudeotide, or using a different modified nudeotide at a given site (e.g., N4_Et_dC instead of t_BB_dC). The mutation detected is indicated in the primer/ probe name. FS# and RS# refer to forward specific primer and reverse specific primer, respectively. CF# and CR# refer to common forward primer and common reverse primer, respectively. F_P# and R_P# refer to forward and reverse probes. While probes in exon 8 are labeled for specific mutations, they can be used to detect any exon 8 mutation; the allele specificity for this assay is determined by allele-specific primers. As an example, for detecting the D538G mutation, any ESRLD538G_FS# primer can be used with any ESRL[Exon8mut]_CR# primer and any ESRL[Exon8mut]_F_P# probe, and any ESRLD538G_RS# primer can be used with any ESRL[Exon8mut]_CF# primer and any ESRL[Exon8mut]_R_P# probe.

Modifications include t_BB_[dNTP]=N6-tert-butyl-benzyl [dNTP]; N4_Et_[dNTP]=N4-Ethyl [dNTP]; N4_Bz_[dNTP]=N4-benzyl [dNTP]; N6_Me_[dNTP]=N6-methyl [dNTP]; pdU=5-propenyl-deoxyuracil; 7_Dz_dG=7-deaza dG; LNA-[dNTP]=locked nucleic acid [dNTP].

TABLE 2
Primer and probe sequences

| SEQ | | | |
|---|---|---|---|
| ID NO | OLIGO NAME | SEQUENCE | MODIFICATION |
| 1 | ESR1_EXWT1_FS03 | TGGCCCTACTGCATCAGATCCAA | |
| 2 | ESR1_EXWT1_FS04 | ATCAGATCCAAGGGAACGAGCT | |
| 3 | ESR1_EXWT1_RS03 | GCTTGCTGCTGTCCAGGTACA | |
| 4 | ESR1_D538G_FS01 | CGTGGTGCCCCTCTATGG | |
| 5 | ESR1_D538G_FS02 | CGTGGTGCCCCTCTATAG | |
| 6 | ESR1_D538G_FS03 | CGTGGTGCCCCTCTATCG | |
| 7 | ESR1_D538G_FS04 | CGTGGTGCCCCTCTATTG | |
| 8 | ESR1_D538G_FS05 | CGTGGTGCCCCTCTAAGG | |
| 9 | ESR1_D538G_FS06 | CGTGGTGCCCCTCTACGG | |
| 10 | ESR1_D538G_FS07 | CGTGGTGCCCCTCTAGGG | |
| 11 | ESR1_D538G_FS08 | CGTGGTGCCCCTCTCTGG | |
| 12 | ESR1_D538G_FS09 | CGTGGTGCCCCTCTGTGG | |
| 13 | ESR1_D538G_FS10 | CGTGGTGCCCCTCTTTGG | |
| 14 | ESR1_D538G_RS01 | GCATCTCCAGCAGC1GGC | 1 = A or optional |
| | | | t_BB_dA |
| 15 | ESR1_D538G_RS02 | GCATCTCCAGCAGCAGAC | |
| 16 | ESR1_D538G_RS03 | GCATCTCCAGCAGCAGCC | |
| 17 | ESR1_D538G_RS04 | GCATCTCCAGCAGCAGTC | |
| 18 | ESR1_D538G_RS05 | GCATCTCCAGCAGCAAGC | |
| 19 | ESR1_D538G_RS06 | GCATCTCCAGCAGCACGC | |
| 20 | ESR1_D538G_RS07 | GCATCTCCAGCAGCATGC | |
| 21 | ESR1_D538G_RS08 | GCATCTCCAGCAG1CGGC | 1 = C or optional |
| | | | N4_Et_dC or |
| | | | t_BB_dC |
| 22 | ESR1_D538G_RS09 | GCATCTCCAGCAG1GGGC | 1 = C or optional |
| | | | N4_Et_dC or |
| | | | t_BB_dC |
| 23 | ESR1_D538G_RS10 | GCATCTCCAGCAG1TGGC | 1 = C or optional |
| | | | N4_Et_dC or |
| | | | t_BB_dC |
| 24 | ESR1_D538G_RS11 | GCATCTCCAGCAGCG1GC | 1 = G or optional |
| | | | 7_Dz_dG |
| 25 | ESR1_D538G_RS12 | CAGCATCTCCAGCAGCTGGC | |
| 26 | ESR1_D538G_TBBDC_RS01 | CAGCATCTCCAGCAG1AGGC | 1 = C or optional |
| | | | t_BB_dC |
| 27 | ESR1_D538G_TBBDC_RS09B | CAGCATCTCCAGCAG1GGGC | 1 = C or optional |
| | | | t_BB_dC |
| 28 | ESR1_E380Q_CF01 | TGAGTCAGCAGGGTTT | |
| 29 | ESR1_E380Q_CR01 | TAGGAGCAAACAGTAGCTTCC | |
| 30 | ESR1_E380Q_CR02 | AGTTAGGAGCAAACAGTAGCTTCC | |
| 31 | ESR1_E380Q_F_P01 | 1TGTGCCT2GGCTAGAGATCCTGATGATTGGT3 | 1 = Reporter |
| | | | 2 = Quencher |
| | | | 3 = Phosphate |
| 32 | ESR1_E380Q_FS01 | CCATGATCAGGTCCACCTT1TAC | 1 = C or optional |
| | | | t_BB_dC |
| 33 | ESR1_E380Q_FS02 | CCATGATCAGGTCCACCTTCTCC | |
| 34 | ESR1_E380Q_FS03 | CCATGATCAGGTCCACCTTCTGC | |
| 35 | ESR1_E380Q_FS04 | CCATGATCAGGTCCACCTTCTTC | |
| 36 | ESR1_E380Q_FS05 | CCATGATCAGGTCCACCTTCAAC | |
| 37 | ESR1_E380Q_FS06 | CCATGATCAGGTCCACCTTCCAC | |
| 38 | ESR1_E380Q_FS07 | CCATGATCAGGTCCACCTTCGAC | |
| 39 | ESR1_E380Q_FS08 | CCATGATCAGGTCCACCTTATAC | |
| 40 | ESR1_E380Q_FS09 | CCATGATCAGGTCCACCTTGTAC | |
| 41 | ESR1_E380Q_FS10 | CCATGATCAGGTCCACCTTTTAC | |
| 42 | ESR1_E380Q_FS11 | CTCCATGATCAGGTCCACCTTGTAC | |
| 43 | ESR1_E380Q_R_P01 | 1TGGACCTG2ATCATGGAGGGTCAAATCCACA3 | 1 = Reporter |
| | | | 2 = Quencher |
| | | | 3 = Phosphate |
| 44 | ESR1_E380Q_RS01 | GGATCTCTAGCCAGGCACATTG | |
| 45 | ESR1_E380Q_RS02 | GGATCTCTAGCCAGGCACATAG | |
| 46 | ESR1_E380Q_RS03 | GGATCTCTAGCCAGGCACATCG | |
| 47 | ESR1_E380Q_RS04 | GGATCTCTAGCCAGGCACATGG | |
| 48 | ESR1_E380Q_RS05 | GGATCTCTAGCCAGGCACAATG | |
| 49 | ESR1_E380Q_RS06 | GGATCTCTAGCCAGGCACACTG | |
| 50 | ESR1_E380Q_RS07 | GGATCTCTAGCCAGGCACAGTG | |
| 51 | ESR1_E380Q_RS08 | GGATCTCTAGCCAGGCACCTTG | |
| 52 | ESR1_E380Q_RS09 | GGATCTCTAGCCAGGCACGTTG | |
| 53 | ESR1_E380Q_RS10 | GGATCTCTAGCCAGGCACTTTG | |
| 54 | ESR1_EXWT1_F_P01 | 1CCCTGAAC2CGTCCGCAGCTCAAGAT3 | 1 = Reporter |
| | | | 2 = Quencher |
| | | | 3 = Phosphate |
| 55 | ESR1_EXWT1_FS01 | CCCTACTGCATCAGATCCAA | |
| 56 | ESR1_EXWT1_FS02 | ATCAGATCCAAGGGAACGAG | |
| 57 | ESR1_EXWT1_RS01 | TTGCTGCTGTCCAGGTACA | |
| 58 | ESR1_L429V_CF01 | TGCTATGTTTTCATAGGAACCAGG | |
| 59 | ESR1_L429V_CR01 | GATTTGAGGCACACAAACTCCT | |
| 60 | ESR1_L429V_F_P01 | 1TCATCTCGGT2TCCGCATGATGAATCTGCAG3 | 1 = Reporter |
| | | | 2 = Quencher |
| | | | 3 = Phosphate |
| 61 | ESR1_L429V_FS01 | TGGAGATCTTCGACATG1TGG | 1 = C or optional |
| | | | N4_Bz_dC or |
| | | | N4_Et_dC or |
| | | | t_BB_dC |
| 62 | ESR1_L429V_FS02 | TGGAGATCTTCGACATGCTAG | |
| 63 | ESR1_L429V_FS03 | TGGAGATCTTCGACATGCTCG | |
| 64 | ESR1_L429V_FS04 | TGGAGATCTTCGACATGCTTG | |
| 65 | ESR1_L429V_FS05 | TGGAGATCTTCGACATGCAGG | |
| 66 | ESR1_L429V_FS06 | TGGAGATCTTCGACATGCCGG | |
| 67 | ESR1_L429V_FS07 | TGGAGATCTTCGACATGCGGG | |
| 68 | ESR1_L429V_FS08 | TGGAGATCTTCGACATGATGG | |
| 69 | ESR1_L429V_FS09 | TGGAGATCTTCGACATGGTGG | |
| 70 | ESR1_L429V_FS10 | TGGAGATCTTCGACATGTTGG | |
| 71 | ESR1_L429V_R_P01 | 1TCGAAGATCTC2CACCATGCCCTCTACACAT3 | 1 = Reporter |
| | | | 2 = Quencher |
| | | | 3 = Phosphate |
| 72 | ESR1_L429V_RS01 | GGAACCGAGATGATGTAGCCAC | |
| 73 | ESR1_L429V_RS02 | GGAACCGAGATGATGTAGCCCC | |
| 74 | ESR1_L429V_RS03 | GGAACCGAGATGATGTAGCCGC | |
| 75 | ESR1_L429V_RS04 | GGAACCGAGATGATGTAGCCTC | |
| 76 | ESR1_L429V_RS05 | GGAACCGAGATGATGTAGCAAC | |
| 77 | ESR1_L429V_RS06 | GGAACCGAGATGATGTAGCGAC | |
| 78 | ESR1_L429V_RS07 | GGAACCGAGATGATGTAGCTAC | |
| 79 | ESR1_L429V_RS08 | GGAACCGAGATGATGTAGACAC | |
| 80 | ESR1_L429V_RS09 | GGAACCGAGATGATGTAGGCAC | |
| 81 | ESR1_L429V_RS10 | GGAACCGAGATGATGTAGTCAC | |
| 82 | ESR1_L536_CF01 | CTTTCTGTGTCTTCCCACCTACAG | |
| 83 | ESR1_L536_CR01 | AAGTGGCTTTGGTCCGTC | |
| 84 | ESR1_L536_D538>P_RS01 | CCAGCAGCAGG1GGG | 1 = G or optional |
| | | | 7_Dz_dG |
| 85 | ESR1_L536_D538>P_RS02 | CAGCAGCAGGG1GG | 1 = G or optional |
| | | | 7_Dz_dG |
| 86 | ESR1_L536_D538>P_RS03 | CCAGCAGCAGG1GG | 1 = G or optional |
| | | | 7_Dz_dG |
| 87 | ESR1_L536_D538>P_RS04 | TCCAGCAGCAGG1GG | 1 = G or optional |
| | | | 7_Dz_dG |
| 88 | ESR1_L536_D538>P_RS05 | CTCCAGCAGCAGG1GG | 1 = G or optional |
| | | | 7_Dz_dG |
| 89 | ESR1_L536_D538>P_RS06 | CTCCAGCAGCAGG1G | 1 = G or optional |
| | | | 7_Dz_dG |
| 90 | ESR1_L536_D538>P_RS07 | TCTCCAGCAGCAGG1G | 1 = G or optional |
| | | | 7_Dz_dG |
| 91 | ESR1_L536_D538>P_RS08 | ATCTCCAGCAGCAGG1G | 1 = G or optional |
| | | | 7_Dz_dG |
| 92 | ESR1_L536_D538>P_RS09 | CATCTCCAGCAGCAGGG | |
| 93 | ESR1_L536_D538>P_RS10 | GCATCTCCAGCAGCAGGG | |
| 94 | ESR1_L536_F_P01 | 1TACATG2CGCCCACTAGCCGTGGA3 | 1 = Reporter |
| | | | 2 = Quencher |
| | | | 3 = Phosphate |
| 95 | ESR1_L536_R_P01 | 1TGCACTT2CATGCTGTACAGATGCTCCATGC3 | 1 = Reporter |
| | | | 2 = Quencher |
| | | | 3 = Phosphate |
| 96 | ESR1_L536H_FS01 | GCAAGAACGTGGTGCCCCA | |
| 97 | ESR1_L536H_FS02 | GCAAGAACGTGGTGCCCAA | |
| 98 | ESR1_L536H_FS03 | GCAAGAACGTGGTGCCCGA | |
| 99 | ESR1_L536H_FS04 | GCAAGAACGTGGTGCCCTA | |
| 100 | ESR1_L536H_FS05 | GCAAGAACGTGGTGCCACA | |
| 101 | ESR1_L536H_FS06 | GCAAGAACGTGGTGCCGCA | |
| 102 | ESR1_L536H_FS07 | GCAAGAACGTGGTGCCTCA | |
| 103 | ESR1_L536H_FS08 | GCAAGAACGTGGTGCACCA | |
| 104 | ESR1_L536H_FS09 | GCAAGAACGTGGTGCGCCA | |
| 105 | ESR1_L536H_FS10 | GCAAGAACGTGGTGCTCCA | |
| 106 | ESR1_L536H_RS01 | CTCCAGCAGCAGGTCAT1GT | 1 = A or optional |
| | | | t_BB_dA |
| 107 | ESR1_L536H_RS02 | CTCCAGCAGCAGGTCATAAT | |
| 108 | ESR1_L536H_RS03 | CTCCAGCAGCAGGTCATACT | |
| 109 | ESR1_L536H_RS04 | CTCCAGCAGCAGGTCATATT | |
| 110 | ESR1_L536H_RS05 | CTCCAGCAGCAGGTCATCGT | |
| 111 | ESR1_L536H_RS06 | CTCCAGCAGCAGGTCATGGT | |
| 112 | ESR1_L536H_RS07 | CTCCAGCAGCAGGTCATTGT | |
| 113 | ESR1_L536H_RS08 | CTCCAGCAGCAGGTCAAAGT | |
| 114 | ESR1_L536H_RS09 | CTCCAGCAGCAGGTCACAGT | |
| 115 | ESR1_L536H_RS10 | CTCCAGCAGCAGGTCAGAGT | |
| 116 | ESR1_L536H_RS11 | CATCTCCAGCAGCAGGTCATAAT | |
| 117 | ESR1_L536H_RS12 | CATCTCCAGCAGCAGGTCATATT | |
| 118 | ESR1_L536H_RS13 | CATCTCCAGCAGCAGGTCGTAGT | |
| 119 | ESR1_L536H_RS14 | CATCTCCAGCAGCAGGTCTTAGT | |
| 120 | ESR1_L536H_RS15 | CATCTCCAGCAGCAGGTCCTAGT | |
| 121 | ESR1_L536P_FS01 | AGAACGTGGTGCCCCC | |
| 122 | ESR1_L536P_FS02 | AGAACGTGGTGCCCAC | |
| 123 | ESR1_L536P_FS03 | AGAACGTGGTGCCCGC | |
| 124 | ESR1_L536P_FS04 | AGAACGTGGTGCCCTC | |
| 125 | ESR1_L536P_FS05 | AGAACGTGGTGCCACC | |
| 126 | ESR1_L536P_FS06 | AGAACGTGGTGCCGCC | |
| 127 | ESR1_L536P_FS07 | AGAACGTGGTGCCTCC | |
| 128 | ESR1_L536P_FS08 | AGAACGTGGTGCACCC | |
| 129 | ESR1_L536P_FS09 | AGAACGTGGTGCGCCC | |
| 130 | ESR1_L536P_FS10 | AGAACGTGGTGCTCCC | |
| 131 | ESR1_L536P_RS01 | CTCCAGCAGCAGGTCAT1GG | 1 = A or optional |
| | | | t_BB_dA |
| 132 | ESR1_L536P_RS02 | CTCCAGCAGCAGGTCATAAG | |
| 133 | ESR1_L536P_RS03 | CTCCAGCAGCAGGTCATACG | |
| 134 | ESR1_L536P_RS04 | CTCCAGCAGCAGGTCATATG | |
| 135 | ESR1_L536P_RS05 | CTCCAGCAGCAGGTCATCGG | |
| 136 | ESR1_L536P_RS06 | CTCCAGCAGCAGGTCATGGG | |
| 137 | ESR1_L536P_RS07 | CTCCAGCAGCAGGTCATTGG | |
| 138 | ESR1_L536P_RS08 | CTCCAGCAGCAGGTCAAAGG | |
| 139 | ESR1_L536P_RS09 | CTCCAGCAGCAGGTCACAGG | |
| 140 | ESR1_L536P_RS10 | CTCCAGCAGCAGGTCAGAGG | |
| 141 | ESR1_L536Q_FS01 | AGAACGTGGTGCCCCAG | |
| 142 | ESR1_L536Q_FS02 | AGAACGTGGTGCCCCCG | |
| 143 | ESR1_L536Q_FS03 | AGAACGTGGTGCCCCGG | |
| 144 | ESR1_L536Q_FS04 | AGAACGTGGTGCCCCTG | |
| 145 | ESR1_L536Q_FS05 | AGAACGTGGTGCCCAAG | |
| 146 | ESR1_L536Q_FS06 | AGAACGTGGTGCCCGAG | |
| 147 | ESR1_L536Q_FS07 | AGAACGTGGTGCCCTAG | |
| 148 | ESR1_L536Q_FS08 | AGAACGTGGTGCCACAG | |
| 149 | ESR1_L536Q_FS09 | AGAACGTGGTGCCGCAG | |
| 150 | ESR1_L536Q_FS10 | AGAACGTGGTGCCTCAG | |
| 151 | ESR1_L536Q_RS01 | CTCCAGCAGCAGGTCAT1CT | 1 = A or optional |
| | | | t_BB_dA |
| 152 | ESR1_L536Q_RS02 | CTCCAGCAGCAGGTCATAAT | |
| 153 | ESR1_L536Q_RS03 | CTCCAGCAGCAGGTCATAGT | |
| 154 | ESR1_L536Q_RS04 | CTCCAGCAGCAGGTCATATT | |
| 155 | ESR1_L536Q_RS05 | CTCCAGCAGCAGGTCATCCT | |
| 156 | ESR1_L536Q_RS06 | CTCCAGCAGCAGGTCATGCT | |
| 157 | ESR1_L536Q_RS07 | CTCCAGCAGCAGGTCATTCT | |
| 158 | ESR1_L536Q_RS08 | CTCCAGCAGCAGGTCAAACT | |
| 159 | ESR1_L536Q_RS09 | CTCCAGCAGCAGGTCACACT | |
| 160 | ESR1_L536Q_RS10 | CTCCAGCAGCAGGTCAGACT | |
| 161 | ESR1_L536R_FS01 | AGAACGTGGTGCCCCG | |
| 162 | ESR1_L536R_FS02 | AGAACGTGGTGCCCAG | |
| 163 | ESR1_L536R_FS03 | AGAACGTGGTGCCCGG | |
| 164 | ESR1_L536R_FS04 | AGAACGTGGTGCCCTG | |
| 165 | ESR1_L536R_FS05 | AGAACGTGGTGCCACG | |
| 166 | ESR1_L536R_FS06 | AGAACGTGGTGCCGCG | |
| 167 | ESR1_L536R_FS07 | AGAACGTGGTGCCTCG | |
| 168 | ESR1_L536R_FS08 | AGAACGTGGTGCACCG | |
| 169 | ESR1_L536R_FS09 | AGAACGTGGTGCGCCG | |
| 170 | ESR1_L536R_FS10 | AGAACGTGGTGCTCCG | |
| 171 | ESR1_L536R_RS01 | CTCCAGCAGCAGGTCAT1GC | 1 = A or optional |
| | | | t_BB_dA |
| 172 | ESR1_L536R_RS02 | CTCCAGCAGCAGGTCATAAC | |
| 173 | ESR1_L536R_RS03 | CTCCAGCAGCAGGTCATACC | |
| 174 | ESR1_L536R_RS04 | CTCCAGCAGCAGGTCATATC | |
| 175 | ESR1_L536R_RS05 | CTCCAGCAGCAGGTCATCGC | |
| 176 | ESR1_L536R_RS06 | CTCCAGCAGCAGGTCATGGC | |
| 177 | ESR1_L536R_RS07 | CTCCAGCAGCAGGTCATTGC | |
| 178 | ESR1_L536R_RS08 | CTCCAGCAGCAGGTCAAAGC | |
| 179 | ESR1_L536R_RS09 | CTCCAGCAGCAGGTCACAGC | |
| 180 | ESR1_L536R_RS10 | CTCCAGCAGCAGGTCAGAGC | |
| 181 | ESR1_P535H_FS01 | AAGTGCAAGAACGTGGTGC1 | 1 = A or optional |
| | | | N6_Bz_dA |
| 182 | ESR1_P535H_FS02 | AAGTGCAAGAACGTGGTGAA | |
| 183 | ESR1_P535H_FS03 | AAGTGCAAGAACGTGGTGGA | |
| 184 | ESR1_P535H_FS04 | AAGTGCAAGAACGTGGTGTA | |
| 185 | ESR1_P535H_FS05 | AAGTGCAAGAACGTGGTACA | |
| 186 | ESR1_P535H_FS06 | AAGTGCAAGAACGTGGTCCA | |
| 187 | ESR1_P535H_FS07 | AAGTGCAAGAACGTGGTTCA | |
| 188 | ESR1_P535H_FS08 | AAGTGCAAGAACGTGGAGCA | |
| 189 | ESR1_P535H_FS09 | AAGTGCAAGAACGTGGCGCA | |
| 190 | ESR1_P535H_FS10 | AAGTGCAAGAACGTGGGGCA | |
| 191 | ESR1_P535H_RS01 | AGCAGCAGGTCATAGAGGT | |
| 192 | ESR1_P535H_RS02 | AGCAGCAGGTCATAGAGAT | |
| 193 | ESR1_P535H_RS03 | AGCAGCAGGTCATAGAGCT | |
| 194 | ESR1_P535H_RS04 | AGCAGCAGGTCATAGAGTT | |
| 195 | ESR1_P535H_RS05 | AGCAGCAGGTCATAGAAGT | |
| 196 | ESR1_P535H_RS06 | AGCAGCAGGTCATAGACGT | |
| 197 | ESR1_P535H_RS07 | AGCAGCAGGTCATAGATGT | |
| 198 | ESR1_P535H_RS08 | AGCAGCAGGTCATAGCGGT | |
| 199 | ESR1_P535H_RS09 | AGCAGCAGGTCATAGGGGT | |
| 200 | ESR1_P535H_RS10 | AGCAGCAGGTCATAGTGGT | |
| 201 | ESR1_P535H_RS11 | CCAGCAGCAGGTCAT1GAGGT | 1 = A or optional |
| | | | t_BB_dA |
| 202 | ESR1_P535H_RS12 | CCAGCAGCAGGTCATG1GGT | 1 = A or C or |
| | | | optional t_BB_dA |
| | | | or t_BB_dC |
| 203 | ESR1_P535H_RS13 | CCAGCAGCAGGTCATAGGGT | |
| 204 | ESR1_P535H_RS14 | CCAGCAGCAGGTCAT1GTGGT | 1 = A or optional |
| | | | t_BB_dA |
| 205 | ESR1_P535H_RS15 | AGCAGCAGGTCATAAAGGT | |
| 206 | ESR1_P535H_RS16 | AGCAGCAGGTCATACAGGT | |
| 207 | ESR1_P535H_RS17 | AGCAGCAGGTCATATAGGT | |
| 208 | ESR1_P535H_RS18 | AGCAGCAGGTCATAGAGG1 | 1 = Optional LNA-T |
| 209 | ESR1_P535H_RS19 | AGCAGCAGGTCATAGAG1T | 1 = Optional LNA-G |
| 210 | ESR1_P535H_RS20 | AGCAGCAGGTCATAGA1GT | 1 = Optional LNA-G |
| 211 | ESR1_P535H_RS21 | AGCAGCAGGTCATAG1GGT | 1 = Optional LNA-A |
| 212 | ESR1_P535H_RS22 | CCAGCAGCAGGTCATAG1GGT | 1 = C or optional |
| | | | N4_Et_dC |
| 213 | ESR1_P535H_RS23 | AGCAGCAGGTCATAAACGT | |
| 214 | ESR1_P535H_RS24 | AGCAGCAGGTCATACACGT | |
| 215 | ESR1_P535H_RS25 | AGCAGCAGGTCATATACGT | |
| 216 | ESR1_P535H_RS26 | AGCAGCAGGTCATAAATGT | |
| 217 | ESR1_P535H_RS27 | AGCAGCAGGTCATACATGT | |
| 218 | ESR1_P535H_RS28 | AGCAGCAGGTCATATATGT | |
| 219 | ESR1_P535H_RS29 | AGCAGCAGGTCATAAAAGT | |
| 220 | ESR1_P535H_RS30 | AGCAGCAGGTCATACAAGT | |
| 221 | ESR1_P535H_RS31 | AGCAGCAGGTCATATAAGT | |
| 222 | ESR1_P535H_RS32 | AGCAGCAGGTCAT1GAGGT | 1 = A or optional |
| | | | N6_Me_dA |
| 223 | ESR1_P535H_RS33 | CCAGCAGCAGGTCATAG1GGT | 1 = A or optional |
| | | | N6_Me_dA |
| 224 | ESR1_P535H_RS34 | CCAGCAGCAGGTCAT1GTGGT | 1 = A or optional |
| | | | N6_Me_dA |
| 225 | ESR1_P535H_RS35 | AGCAGCAGGTCATAG1GGT | 1 = A or optional |
| | | | t_BB_dA |
| 226 | ESR1_P535H_RS36 | CAGCAGCAGGTCATAAAGGT | |
| 227 | ESR1_P535H_RS37 | CAGCAGCAGGTCATACAGGT | |
| 228 | ESR1_P535H_RS38 | CAGCAGCAGGTCATATAGGT | |
| 229 | ESR1_P535H_RS39 | CCAGCAGCAGGTCATAAAGGT | |
| 230 | ESR1_P535H_RS40 | CCAGCAGCAGGTCATACAGGT | |
| 231 | ESR1_P535H_RS41 | CCAGCAGCAGGTCATATAGGT | |
| 232 | ESR1_S341L_CF01 | AGATGGTCAGTGCCTTGTTG | |
| 233 | ESR1_S341L_CF02 | CAGATGGTCAGTGCCTTGTT | |
| 234 | ESR1_S341L_CR01 | ATTCTTACCTGGCACCCTCTTC | |
| 235 | ESR1_S341L_CR02 | CTCTTCGCCCAGTTGATCAT | |
| 236 | ESR1_S341L_F_P01 | 1AGACAGG2GAGCTGGTTCACATGATCAACTG3 | 1 = Reporter |
| | | | 2 = Quencher |
| | | | 3 = Phosphate |
| 237 | ESR1_S341L_F_P02 | 1TGATGG2GCTTACTGACCAACCTGGCAGACA3 | 1 = Reporter |
| | | | 2 = Quencher |
| | | | 3 = Phosphate |
| 238 | ESR1_S341L_FS01 | CCAGACCCTTCAGTGAAG1TTT | 1 = C or optional |
| | | | t_BB_dC |
| 239 | ESR1_S341L_FS02 | CCAGACCCTTCAGTGAAGCTAT | |
| 240 | ESR1_S341L_FS03 | CCAGACCCTTCAGTGAAGCTCT | |
| 241 | ESR1_S341L_FS04 | CCAGACCCTTCAGTGAAGCTGT | |
| 242 | ESR1_S341L_FS05 | CCAGACCCTTCAGTGAAGCATT | |
| 243 | ESR1_S341L_FS06 | CCAGACCCTTCAGTGAAGCCTT | |
| 244 | ESR1_S341L_FS07 | CCAGACCCTTCAGTGAAGCGTT | |
| 245 | ESR1_S341L_FS08 | CCAGACCCTTCAGTGAAGATTT | |
| 246 | ESR1_S341L_FS09 | CCAGACCCTTCAGTGAAGGTTT | |
| 247 | ESR1_S341L_FS10 | CCAGACCCTTCAGTGAAGTTTT | |
| 248 | ESR1_S341L_R_P01 | 1ACTGAAG2GGTCTGGTAGGATCATACTCGGA3 | |
| 249 | ESR1_S341L_R_P02 | 1CTGAAG23GTCTGGTAGGATCATACTCGGAATA4 | 1 = Reporter |
| | | | 2 = Quencher |
| | | | 3 = G or optional |
| | | | 7_Dz_dG |
| | | | 4 = Phosphate |
| 250 | ESR1_S341L_RS01 | GTTGGTCAGTAAGCCCATCATCA | |
| 251 | ESR1_S341L_RS02 | GTTGGTCAGTAAGCCCATCATAA | |
| 252 | ESR1_S341L_RS03 | GTTGGTCAGTAAGCCCATCATGA | |
| 253 | ESR1_S341L_RS04 | GTTGGTCAGTAAGCCCATCATTA | |
| 254 | ESR1_S341L_RS05 | GTTGGTCAGTAAGCCCATCAACA | |
| 255 | ESR1_S341L_RS06 | GTTGGTCAGTAAGCCCATCACCA | |
| 256 | ESR1_S341L_RS07 | GTTGGTCAGTAAGCCCATCAGCA | |
| 257 | ESR1_S341L_RS08 | GTTGGTCAGTAAGCCCATCCTCA | |
| 258 | ESR1_S341L_RS09 | GTTGGTCAGTAAGCCCATCGTCA | |
| 259 | ESR1_S341L_RS10 | GTTGGTCAGTAAGCCCATCTTCA | |
| 260 | ESR1_S463P_CF01 | CTAGACCTCATCCTCTTTGAGC | |
| 261 | ESR1_S463P_CR01 | CCATCAGGTGGATCAAAGTGTCTG | |
| 262 | ESR1_S463P_F_P01 | 1ACCATAT2CCACCGAGTCCTGGACAAGATCA3 | 1 = Reporter |
| | | | 2 = Quencher |
| | | | 3 = Phosphate |
| 263 | ESR1_S463P_FS01 | CATTCAGGAGTGTACACATTT1TGC | 1 = C or optional |
| | | | t_BB_dC |
| 264 | ESR1_S463P_FS02 | CATTCAGGAGTGTACACATTTCTAC | |
| 265 | ESR1_S463P_FS03 | CATTCAGGAGTGTACACATTTCTCC | |
| 266 | ESR1_S463P_FS04 | CATTCAGGAGTGTACACATTTCTTC | |
| 267 | ESR1_S463P_FS05 | CATTCAGGAGTGTACACATTTCAGC | |
| 268 | ESR1_S463P_FS06 | CATTCAGGAGTGTACACATTTCCGC | |
| 269 | ESR1_S463P_FS07 | CATTCAGGAGTGTACACATTTCGGC | |
| 270 | ESR1_S463P_FS08 | CATTCAGGAGTGTACACATTTATGC | |
| 271 | ESR1_S463P_FS09 | CATTCAGGAGTGTACACATTTGTGC | |
| 272 | ESR1_S463P_FS10 | CATTCAGGAGTGTACACATTTTTGC | |
| 273 | ESR1_S463P_R_P01 | 1TGTGTAC2ACTCCTGAATGCGCAGAGAGAGA3 | 1 = Reporter |
| | | | 2 = Quencher |
| | | | 3 = Phosphate |
| 274 | ESR1_S463P_RS01 | AGACTTCAGGGTGCTGGG | |
| 275 | ESR1_S463P_RS11 | CAGACTTCAGGGTGCTGAG | |
| 276 | ESR1_S463P_RS03 | AGACTTCAGGGTGCTGCG | |
| 277 | ESR1_S463P_RS04 | AGACTTCAGGGTGCTGTG | |
| 278 | ESR1_S463P_RS05 | AGACTTCAGGGTGCTAGG | |
| 279 | ESR1_S463P_RS06 | AGACTTCAGGGTGCTCGG | |
| 280 | ESR1_S463P_RS07 | AGACTTCAGGGTGCTTGG | |
| 281 | ESR1_S463P_RS08 | AGACTTCAGGGTGCAGGG | |
| 282 | ESR1_S463P_RS09 | AGACTTCAGGGTGCCGGG | |
| 283 | ESR1_S463P_RS10 | AGACTTCAGGGTGCGGGG | |
| 284 | ESR1_V422DELV_CF01 | GTCTTGTGGAAGATTTTCTGT | |
| 285 | ESR1_V422DELV_CR01 | TTGAGGCACACAAACTCCTC | |
| 286 | ESR1_V422DELV_F_P01 | 1TGGCTACATCA2TCTCGGTTCCGCATGATGA3 | 1 = Reporter |
| | | | 2 = Quencher |
| | | | 3 = Phosphate |
| 287 | ESR1_V422DELV_FS01 | ATGTGTAGAGGGCATGG1GAT | 1 = A or optional |
| | | | t_BB_dA |
| 288 | ESR1_V422DELV_FS02 | ATGTGTAGAGGGCATGGAGCT | |
| 289 | ESR1_V422DELV_FS03 | ATGTGTAGAGGGCATGGAGGT | |
| 290 | ESR1_V422DELV_FS04 | ATGTGTAGAGGGCATGGAGTT | |
| 291 | ESR1_V422DELV_FS05 | ATGTGTAGAGGGCATGGAAAT | |
| 292 | ESR1_V422DELV_FS06 | ATGTGTAGAGGGCATGGACAT | |
| 293 | ESR1_V422DELV_FS07 | ATGTGTAGAGGGCATGGATAT | |
| 294 | ESR1_V422DELV_FS08 | ATGTGTAGAGGGCATGGCGAT | |
| 295 | ESR1_V422DELV_FS09 | ATGTGTAGAGGGCATG12GAT | 1 = G or optional |
| | | | 7_Dz_dG |
| | | | 2 = G or optional |
| | | | 7_Dz_dG |
| 296 | ESR1_V422DELV_FS10 | ATGTGTAGAGGGCATGGTGAT | |
| 297 | ESR1_V422DELV_FS14 | AAATGTGTAGAG1GCATGGCGAT | 1 = G or optional |
| | | | d_I |
| 298 | ESR1_V422DELV_FS15 | AAATGTGTAGAGGGCATGGTGAT | |
| 299 | ESR1_V422DELV_FS16 | AAATGTGTAGAGGGCATGTAGAT | |
| 300 | ESR1_V422DELV_FS17 | AAATGTGTAGAGGGCATGAAGAT | |
| 301 | ESR1_V422DELV_R_P01 | 1CCTCTACACATT2TTCCCTGGTTCCTATGA3 | 1 = Reporter |
| | | | 2 = Quencher |
| | | | 3 = Phosphate |
| 302 | ESR1_V422DELV_RS01 | GCAGCATGTCGAAGATCTCCAT | |
| 303 | ESR1_V422DELV_RS02 | GCAGCATGTCGAAGATCTCCCT | |
| 304 | ESR1_V422DELV_RS03 | GCAGCATGTCGAAGATCTCCGT | |
| 305 | ESR1_V422DELV_RS04 | GCAGCATGTCGAAGATCTCCTT | |
| 306 | ESR1_V422DELV_RS05 | GCAGCATGTCGAAGATCTCAAT | |
| 307 | ESR1_V422DELV_RS06 | GCAGCATGTCGAAGATCTCGAT | |
| 308 | ESR1_V422DELV_RS07 | GCAGCATGTCGAAGATCTCTAT | |
| 309 | ESR1_V422DELV_RS08 | GCAGCATGTCGAAGATCTACAT | |
| 310 | ESR1_V422DELV_RS09 | GCAGCATGTCGAAGATCTGCAT | |
| 311 | ESR1_V422DELV_RS10 | GCAGCATGTCGAAGATCTTCAT | |
| 312 | ESR1_V533M_CF01 | GTAGTCCTTTCTGTGTCTTCCC | |
| 313 | ESR1_V533M_CF02 | CTTTCTGTGTCTTCCCACCTAC | |
| 314 | ESR1_V533M_CF03 | TGTCTTCCCACCTACAGTAACAAA | |
| 315 | ESR1_V533M_CF04 | CTCTAAAGTAGTCCTTTCTGTGTCTTC | |
| 316 | ESR1_V533M_CF05 | TCTAAAGTAGTCCTTTCTGTGTCTTC | |
| 317 | ESR1_V533M_CF06 | TAAAGTAGTCCTTTCTGTGTCTTCC | |
| 318 | ESR1_V533M_CF07 | AGTAGTCCTTTCTGTGTCTTCC | |
| 319 | ESR1_V533M_CR01 | GCTAGTGGGCGCATGTA | |
| 320 | ESR1_V533M_CR02 | CTAGTGGGCGCATGTA | |
| 321 | ESR1_V533M_F_P01 | 1TCTATG2ACCTGCTGCTGGAGATGCTGGA3 | 1 = Reporter |
| | | | 2 = Quencher |
| | | | 3 = Phosphate |
| 322 | ESR1_V533M_FS01 | ACAGCATGAAGTGCAAGAA12 | 1 = C or optional |
| | | | N4_Bz_dC or |
| | | | N4_Et_dC |
| | | | 2 = A or optional |
| | | | N6_Bz_dA |
| 323 | ESR1_V533M_FS02 | ACAGCATGAAGTGCAAGAAAA | |
| 324 | ESR1_V533M_FS03 | ACAGCATGAAGTGCAAGAAGA | |
| 325 | ESR1_V533M_FS04 | ACAGCATGAAGTGCAAGAATA | |
| 326 | ESR1_V533M_FS05 | ACAGCATGAAGTGCAAGACCA | |
| 327 | ESR1_V533M_FS06 | ACAGCATGAAGTGCAAGAGCA | |
| 328 | ESR1_V533M_FS07 | ACAGCATGAAGTGCAAGATCA | |
| 329 | ESR1_V533M_FS08 | ACAGCATGAAGTGCAAGCACA | |
| 330 | ESR1_V533M_FS09 | ACAGCATGAAGTGCAAGGACA | |
| 331 | ESR1_V533M_FS10 | ACAGCATGAAGTGCAAGTACA | |
| 332 | ESR1_V533M_FS12 | TGTACAGCATGAAGTGCAAGCACA | |
| 333 | ESR1_V533M_FS13 | TGTACAGCATGAAGTGCAAGGACA | |
| 334 | ESR1_V533M_R_P01 | 1TGCACT2TCATGCTGTACAGATGCTCCATGC3 | 1 = Reporter |
| | | | 2 = Quencher |
| | | | 3 = Phosphate |
| 335 | ESR1_V533M_R_P02 | 1TGCACTT2CATGCTGTACAGATGCTCCATGC3 | 1 = Reporter |
| | | | 2 = Quencher |
| | | | 3 = Phosphate |
| 336 | ESR1_V533M_RS01 | GGTCATAGAG1GGCACCAT | 1 = Optional |
| | | | 7_Dz_dG |
| 337 | ESR1_V533M_RS018 | GGTCATAGAG1GGCA2CAT | 1 = Optional |
| | | | 7_Dz_dG |
| | | | 2 = C or optional |
| | | | t_BB_dC or |
| | | | N4_Et_dC |
| 338 | ESR1_V533M_RS02 | GGTCATAGAG1GGCACCCT | 1 = Optional |
| | | | 7_Dz_dG |
| 339 | ESR1_V533M_RS20 | GGTCATAGAG1GGCACC2T | 1 = Optional |
| | | | 7_Dz_dG |
| | | | 2 = C or optional |
| | | | LNA-A |
| 340 | ESR1_V533M_RS03 | GGTCATAGAG1GGCACCGT | 1 = Optional |
| | | | 7_Dz_dG |
| 341 | ESR1_V533M_RS04 | GGTCATAGAG1GGCACCTT | 1 = Optional |
| | | | 7_Dz_dG |
| 342 | ESR1_V533M_RS05 | GGTCATAGAG1GGCACAAT | 1 = Optional |
| | | | 7_Dz_dG |
| 343 | ESR1_V533M_RS06 | GGTCATAGAG1GGCACGAT | 1 = Optional |
| | | | 7_Dz_dG |
| 344 | ESR1_V533M_RS07 | GGTCATAGAG1GGCACTAT | 1 = Optional |
| | | | 7_Dz_dG |
| 345 | ESR1_V533M_RS08 | GGTCATAGAG1GGCAACAT | 1 = Optional |
| | | | 7_Dz_dG |
| 346 | ESR1_V533M_RS09 | GGTCATAGAG1GGCAGCAT | 1 = Optional |
| | | | 7_Dz_dG |
| 347 | ESR1_V533M_RS10 | GGTCATAGAG1GGCATCAT | 1 = Optional |
| | | | 7_Dz_dG |
| 348 | ESR1_V533M_RS11 | AGGTCATAGAG1GGCAGCAT | 1 = Optional |
| | | | 7_Dz_dG |
| 349 | ESR1_V533M_RS12 | AGGTCATAGAG1GGCTCCAT | 1 = Optional |
| | | | 7_Dz_dG |
| 350 | ESR1_V533M_RS13 | AGGTCATAGAG1GGCGCCAT | 1 = Optional |
| | | | 7_Dz_dG |
| 351 | ESR1_V533M_RS14 | AGGTCATAGAG1GGCCCCAT | 1 = Optional |
| | | | 7_Dz_dG |
| 352 | ESR1_V533M_RS15 | CAGGTCATAGAG1GGCTCCAT | 1 = Optional |
| | | | 7_Dz_dG |
| 353 | ESR1_V533M_RS16 | CAGGTCATAGAG1GGCGCCAT | 1 = Optional |
| | | | 7_Dz_dG |
| 354 | ESR1_V533M_RS17 | CAGGTCATAGAG1GGCCCCAT | 1 = Optional |
| | | | 7_Dz_dG |
| 355 | ESR1_V533M_TBBA_FS01 | ACAGCATGAAGTGCAAG1ACA | 1 = A or optional |
| | | | t_BB_dA |
| 356 | ESR1_V533M_TBBDC_FS01 | ACAGCATGAAGTGCAAGAA1A | 1 = C or optional |
| | | | t_BB_dC |
| 357 | ESR1_V534E_CF01 | CTTTCTGTGTCTTCCCACCTAC | |
| 358 | ESR1_V534E_CRO1 | GCTTTGGTCCGTCTCCT | |
| 359 | ESR1_V534E_CR02 | TGGCTTTGGTCCGTCTCCT | |
| 360 | ESR1_V534E_CR03 | ATGTAGGCGGTGGGCGTC | |
| 361 | ESR1_V534E_CR04 | CTCCACGGCTAGTGGGCG | |
| 362 | ESR1_V534E_CR05 | TGCCCCTCCACGGCTAGT | |
| 363 | ESR1_V534E_F_P01 | 1TCTATGA2CCTGCTGCTGGAGATGCTGGA3 | 1 = Reporter |
| | | | 2 = Quencher |
| | | | 3 = Phophate |
| 364 | ESR1_V534E_FS01 | GCATGAAGTGCAAGAACGTGG1 | 1 = A or optional |
| | | | N6_Bz_dA |
| 365 | ESR1_V534E_FS02 | GCATGAAGTGCAAGAACGTGAA | |
| 366 | ESR1_V534E_FS03 | GCATGAAGTGCAAGAACGTGCA | |
| 367 | ESR1_V534E_FS04 | GCATGAAGTGCAAGAACGTGTA | |
| 368 | ESR1_V534E_FS05 | GCATGAAGTGCAAGAACGTAGA | |
| 369 | ESR1_V534E_FS06 | GCATGAAGTGCAAGAACGTCGA | |
| 370 | ESR1_V534E_FS07 | GCATGAAGTGCAAGAACGTTGA | |
| 371 | ESR1_V534E_FS08 | GCATGAAGTGCAAGAACGAGGA | |
| 372 | ESR1_V534E_FS09 | GCATGAAGTGCAAGAACGCGGA | |
| 373 | ESR1_V534E_FS10 | GCATGAAGTGCAAGAACGGGGA | |
| 374 | ESR1_V534E_R_P01 | 1TGCACTT2CATGCTGTACAGATGCTCCATG3 | 1 = Reporter |
| | | | 2 = Quencher |
| | | | 3 = Phosphate |
| 375 | ESR1_V534E_R_P03 | 1TGCACTT2CA3GCTG3ACAGATGC3CCATG4 | 1 = Reporter |
| | | | 2 = Quencher |
| | | | 3 = pdU(3x) |
| | | | 4 = Phosphate |
| 376 | ESR1_V534E_R_P04 | 1TGCACTT2CATGCTGTACAGATGCTCCAT3 | 1 = Reporter |
| | | | 2 = Quencher |
| | | | 3 = Phosphate |
| 377 | ESR1_V534E_R_P05 | 1TGCACTT2CA3GCTG3ACAGA3GCTCCA34 | 1 = Reporter |
| | | | 2 = Quencher |
| | | | 3 = pdU(4x) |
| | | | 4 = Phosphate |
| 378 | ESR1_V534E_RS01 | GCAGGTCATAGAG1GGCT | 1 = G or optional |
| | | | 7_Dz_dG |
| 379 | ESR1_V534E_RS02 | GCAGGTCATAGAGGGGAT | |
| 380 | ESR1_V534E_RS03 | GCAGGTCATAGAGGGGGT | |
| 381 | ESR1_V534E_RS04 | GCAGGTCATAGAGGGGTT | |
| 382 | ESR1_V534E_RS05 | GCAGGTCATAGAGGGACT | |
| 383 | ESR1_V534E_RS06 | GCAGGTCATAGAGGGCCT | |
| 384 | ESR1_V534E_RS07 | GCAGGTCATAGAGGGTCT | |
| 385 | ESR1_V534E_RS08 | GCAGGTCATAGAGGAGCT | |
| 386 | ESR1_V534E_RS09 | GCAGGTCATAGAGGCGCT | |
| 387 | ESR1_V534E_RS10 | GCAGGTCATAGAGGTGCT | |
| 388 | ESR1_V534E_RS11 | GCAGGTCATAGAG1GGCT | 1 = G or optional |
| | | | 7_Dz_dG |
| 389 | ESR1_V534E_RS13 | GCAGGTCATAGAGTGGCT | |
| 390 | ESR1_V534E_RS14 | GCAGGTCATAGAGCGGCT | |
| 391 | ESR1_V534E_RS15 | CAGCAGGTCATAGAGAGGCT | |
| 392 | ESR1_V534E_RS16 | CAGCAGGTCATAGAGTGGCT | |
| 393 | ESR1_V534E_RS17 | CAGCAGGTCATAGAGCGGCT | |
| 394 | ESR1_Y537C_CF01 | CTGTGTCTTCCCACCTACAGTA | |
| 395 | ESR1_Y537C_CR01 | AAGTGGCTTTGGTCCGT | |
| 396 | ESR1_Y537C_F_P01 | 1TACATG2CGCCCACTAGCCGTGGA3 | 1 = Reporter |
| | | | 2 = Quencher |
| | | | 3 = Phosphate |
| 397 | ESR1_Y537C_FS01 | ACGTGGTGCCCCTCTG | |
| 398 | ESR1_Y537C_FS02 | ACGTGGTGCCCCTCAG | |
| 399 | ESR1_Y537C_FS03 | ACGTGGTGCCCCTCCG | |
| 400 | ESR1_Y537C_FS04 | ACGTGGTGCCCCTCGG | |
| 401 | ESR1_Y537C_FS05 | ACGTGGTGCCCCTATG | |
| 402 | ESR1_Y537C_FS06 | ACGTGGTGCCCCTGTG | |
| 403 | ESR1_Y537C_FS07 | ACGTGGTGCCCCTTTG | |
| 404 | ESR1_Y537C_FS08 | ACGTGGTGCCCCACTG | |
| 405 | ESR1_Y537C_FS09 | ACGTGGTGCCCCCCTG | |
| 406 | ESR1_Y537C_FS10 | ACGTGGTGCCCCGCTG | |
| 407 | ESR1_Y537C_R_P01 | 1TGCACTT2CATGCTGTACAGATGCTCCATGC3 | 1 = Reporter |
| | | | 2 = Quencher |
| | | | 3 = Phosphate |
| 408 | ESR1_Y537C_RS01 | CTCCAGCAGCAGGT1AC | 1 = C or optional |
| | | | t_BB_dC |
| 409 | ESR1_Y537C_RS02 | CTCCAGCAGCAGGTCCC | |
| 410 | ESR1_Y537C_RS03 | CTCCAGCAGCAGGTCGC | |
| 411 | ESR1_Y537C_RS04 | CTCCAGCAGCAGGTCTC | |
| 412 | ESR1_Y537C_RS05 | CTCCAGCAGCAGGTAAC | |
| 413 | ESR1_Y537C_RS06 | CTCCAGCAGCAGGTGAC | |
| 414 | ESR1_Y537C_RS07 | CTCCAGCAGCAGGTTAC | |
| 415 | ESR1_Y537C_RS08 | CTCCAGCAGCAGGACAC | |
| 416 | ESR1_Y537C_RS09 | CTCCAGCAGCAGGCCAC | |
| 417 | ESR1_Y537C_RS10 | CTCCAGCAGCAGGGCAC | |
| 418 | ESR1_Y537C_RS11 | ATCTCCAGCAGCAGGACAC | |
| 419 | ESR1_Y537C_RS12 | CATCTCCAGCAGCAGGACAC | |
| 420 | ESR1_Y537C_RS13 | ATCTCCAGCAGCAGGT1AC | 1 = C or optional |
| | | | t_BB_dC |
| 421 | ESR1_Y537C_RS14 | ATCTCCAGCAGCAGGTC1C | 1 = A or optional |
| | | | t_BB_dA |
| 422 | ESR1_Y537C_RS15 | ATCTCCAGCAGCAGGTCA1 | 1 = C or optional |
| | | | t_BB_dC |
| 423 | ESR1_Y537C_RS16 | CTCCAGCAGCAGGT1AC | 1 = C or optional |
| | | | t_BB_dC |
| 424 | ESR1_Y537C_RS17 | CTCCAGCAGCAGGTC1C | 1 = A or optional |
| | | | t_BB_dA or |
| | | | LNA-A |
| 425 | ESR1_Y537C_RS18 | CTCCAGCAGCAGGTCA1 | 1 = C or optional |
| | | | t_BB_dC |
| 426 | ESR1_Y537N_FS01 | AACGTGGTGCCCCTCA | |
| 427 | ESR1_Y537N_FS02 | AACGTGGTGCCCCTAA | |
| 428 | ESR1_Y537N_FS03 | AACGTGGTGCCCCTGA | |
| 429 | ESR1_Y537N_FS04 | AACGTGGTGCCCCTTA | |
| 430 | ESR1_Y537N_FS05 | AACGTGGTGCCCCACA | |
| 431 | ESR1_Y537N_FS06 | AACGTGGTGCCCCCCA | |
| 432 | ESR1_Y537N_FS07 | AACGTGGTGCCCCGCA | |
| 433 | ESR1_Y537N_FS08 | AACGTGGTGCCCATCA | |
| 434 | ESR1_Y537N_FS09 | AACGTGGTGCCCGTCA | |
| 435 | ESR1_Y537N_FS10 | AACGTGGTGCCCTTCA | |
| 436 | ESR1_Y537N_RS01 | CTCCAGCAGCAGGT1ATT | 1 = C or optional |
| | | | t_BB_dC |
| 437 | ESR1_Y537N_RS02 | CTCCAGCAGCAGGTCAAT | |
| 438 | ESR1_Y537N_RS03 | CTCCAGCAGCAGGTCACT | |
| 439 | ESR1_Y537N_RS04 | CTCCAGCAGCAGGTCAGT | |
| 440 | ESR1_Y537N_RS05 | CTCCAGCAGCAGGTCCTT | |
| 441 | ESR1_Y537N_RS06 | CTCCAGCAGCAGGTCGTT | |
| 442 | ESR1_Y537N_RS07 | CTCCAGCAGCAGGTCTTT | |
| 443 | ESR1_Y537N_RS08 | CTCCAGCAGCAGGTAATT | |
| 444 | ESR1_Y537N_RS09 | CTCCAGCAGCAGGTGATT | |
| 445 | ESR1_Y537N_RS10 | CTCCAGCAGCAGGTTATT | |
| 446 | ESR1_Y537N_RS11 | ATCTCCAGCAGCAGGTTATT | |
| 447 | ESR1_Y537N_RS12 | CATCTCCAGCAGCAGGTTATT | |
| 448 | ESR1_Y537N_RS13 | GCATCTCCAGCAGCAGGTTATT | |
| 449 | ESR1_Y537S_FS01 | ACGTGGTGCCCCTCTC | |
| 450 | ESR1_Y537S_FS02 | ACGTGGTGCCCCTCAC | |
| 451 | ESR1_Y537S_FS03 | ACGTGGTGCCCCTCCC | |
| 452 | ESR1_Y537S_FS04 | ACGTGGTGCCCCTCGC | |
| 453 | ESR1_Y537S_FS05 | ACGTGGTGCCCCTATC | |
| 454 | ESR1_Y537S_FS06 | ACGTGGTGCCCCTGTC | |
| 455 | ESR1_Y537S_FS07 | ACGTGGTGCCCCTTTC | |
| 456 | ESR1_Y537S_FS08 | ACGTGGTGCCCCACTC | |
| 457 | ESR1_Y537S_FS09 | ACGTGGTGCCCCCCTC | |
| 458 | ESR1_Y537S_FS10 | ACGTGGTGCCCCGCTC | |
| 459 | ESR1_Y537S_RS01 | ATCTCCAGCAGCAGGT1AG | 1 = C or optional |
| | | | t_BB_dC |
| 460 | ESR1_Y537S_RS02 | ATCTCCAGCAGCAGGTCCG | |
| 461 | ESR1_Y537S_RS03 | ATCTCCAGCAGCAGGTCGG | |
| 462 | ESR1_Y537S_RS04 | ATCTCCAGCAGCAGGTCTG | |
| 463 | ESR1_Y537S_RS05 | ATCTCCAGCAGCAGGTAAG | |
| 464 | ESR1_Y537S_RS06 | ATCTCCAGCAGCAGGTGAG | |
| 465 | ESR1_Y537S_RS07 | ATCTCCAGCAGCAGGTTAG | |
| 466 | ESR1_Y537S_RS08 | ATCTCCAGCAGCAGGACAG | |
| 467 | ESR1_Y537S_RS09 | ATCTCCAGCAGCAGGCCAG | |
| 468 | ESR1_Y537S_RS10 | ATCTCCAGCAGCAGGGCAG | |
| 469 | ESR1_Y537S_RS11 | CATCTCCAGCAGCAGGACAG | |
| 470 | ESR1_Y537S_RS12 | CATCTCCAGCAGCAGGGCAG | |
| 471 | ESR1_Y537S_RS13 | GCATCTCCAGCAGCAGGACAG | |
| 472 | ESR1_Y537S_RS14 | CTCCAGCAGCAGGGCAG | |
| 473 | ESR1K303RWSNP_CF01 | AGAGATGATGGGGAGGGCA | |
| 474 | ESR1K303RWSNP_CF02 | AGATGATGGGGAGGGCA | |
| 475 | ESR1K303RWSNP_CR01 | TCAGCATCCAACAAGGCA | |
| 476 | ESR1K303RWSNP_CR02 | CTCAGCATCCAACAAGGCA | |
| 477 | ESR1K303RWSNP_F_P01 | 1TTGTCCCTGAC2GGCCGACCAGATGGTCA3 | 1 = Reporter |
| | | | 2 = Quencher |
| | | | 3 = Phosphate |
| 478 | ESR1K303RWSNP_FS01 | CGCTCATGATCAAACGCTCTAAGAG | |
| 479 | ESR1K303RWSNP_FS02 | CGCTCATGATCAAACGCTCTAAGCG | |
| 480 | ESR1K303RWSNP_FS03 | CGCTCATGATCAAACGCTCTAAGGG | |
| 481 | ESR1K303RWSNP_FS04 | CGCTCATGATCAAACGCTCTAAGTG | |
| 482 | ESR1K303RWSNP_FS05 | CGCTCATGATCAAACGCTCTAAAAG | |
| 483 | ESR1K303RWSNP_FS06 | CGCTCATGATCAAACGCTCTAACAG | |
| 484 | ESR1K303RWSNP_FS07 | CGCTCATGATCAAACGCTCTAATAG | |
| 485 | ESR1K303RWSNP_FS08 | CGCTCATGATCAAACGCTCTACGAG | |
| 486 | ESR1K303RWSNP_FS09 | CGCTCATGATCAAACGCTCTAGGAG | |
| 487 | ESR1K303RWSNP_FS10 | CGCTCATGATCAAACGCTCTATGAG | |
| 488 | ESR1K303RWSNP_R_P01 | 1TTTGATCATGA2GCGGGCTTGGCCAAAGGTT3 | 1 = Reporter |
| | | | 2 = Quencher |
| | | | 3 = Phosphate |
| 489 | ESR1K303RWSNP_RS01 | ACAAGGCCAGGCTGTTCC | |
| 490 | ESR1K303RWSNP_RS02 | ACAAGGCCAGGCTGTTAC | |
| 491 | ESR1K303RWSNP_RS03 | ACAAGGCCAGGCTGTTGC | |
| 492 | ESR1K303RWSNP_RS04 | ACAAGGCCAGGCTGTTTC | |
| 493 | ESR1K303RWSNP_RS05 | ACAAGGCCAGGCTGTACC | |
| 494 | ESR1K303RWSNP_RS06 | ACAAGGCCAGGCTGTCCC | |
| 495 | ESR1K303RWSNP_RS07 | ACAAGGCCAGGCTGTGCC | |
| 496 | ESR1K303RWSNP_RS08 | ACAAGGCCAGGCTGATCC | |
| 497 | ESR1K303RWSNP_RS09 | ACAAGGCCAGGCTGCTCC | |
| 498 | ESR1K303RWSNP_RS10 | ACAAGGCCAGGCTGGTCC | |
| 499 | SC_ESR1_D01F | GTCTGGCGAGAGATGCAAA | |
| 500 | SC_ESR1_D01FP1 | 1CTCTAC2TTTCCTTACCTCCTTCCTTCCA3 | 1 = Reporter |
| | | | 2 = Quencher |
| | | | 3 = Phosphate |
| 501 | SC_ESR1_D01R | GCCTCAATGAAGACAACTTGAA | |
| 502 | SC_ESR1_D02F | AGGATAAAGTGGATCTGCTGCA | |
| 503 | SC_ESR1_D02R | CCTGGCGTCGATTATCTGAA | |
| 504 | SC_ESR1_D03F | GCTGTTAATTGTCCATGCATAA | |
| 505 | SC_ESR1_D03R | GAAAGGGGAGAACAAGCTAAA | |
| 506 | SC_ESR1_D04F | GAGGAATGGATTTCAATGGAA | |
| 507 | SC_ESR1_D04R | CCCTGGGTCTGTGATCACTAA | |
| 508 | SC_ESR1EX1WTASR1 | GCCACGGACCATGACCATGA | |
| 509 | SC_ESR1EX1WTCRP1 | CTTGAGCTGCGGACGGTTCA | |
| 510 | SC_ESR1EX1WTPRB1 | 1TGGCCCTA2CTGCATCAGATCCAAGG3 | 1 = Reporter |
| | | | 2 = Quencher |
| | | | 3 = Phosphate |
| 511 | SC_ESR1EX2WTASR1 | CAGAGAAAGATTGGCCAGTACC | |
| 512 | SC_ESR1EX2WTASR2 | GCCAGTACCAATGACAAGGGAAG | |
| 513 | SC_ESR1EX2WTASR3 | CCAGGGTGGCAGAGAAAGATT | |
| 514 | SC_ESR1EX2WTCRP1 | CAGACTCCATAATGGTAGCCTGA | |
| 515 | SC_ESR1EX2WTCRP2 | TAATGGTAGCCTGAAGCATAGTCAT | |
| 516 | SC_ESR1EX2WTCRP3 | CACTGCACAGTAGCGAGTCTCCT | |
| 517 | SC_ESR1EX2WTPRB1 | 1TGACAAGG2GAAGTATGGCTATGGAATCT3 | 1 = Reporter |
| | | | 2 = Quencher |
| | | | 3 = Phosphate |
| 518 | SC_ESR1EX2WTPRB2 | 1TGACAAG2G3AAGTATGGCTATGGAATCT4 | 1 = Reporter |
| | | | 2 = 7_Dz_dG |
| | | | 3 = Quencher |
| | | | 4 = Phosphate |
| 519 | SC_ESR1EX2WTPRB2B | 1CTATGGAA2TCTGCCAAGGAGACTCGCTA3 | 1 = Reporter |
| | | | 2 = Quencher |
| | | | 3 = Phosphate |
| 520 | SC_ESR1EX2WTPRB3 | 1TGACAAGGGA2AGTATGGCTATGGAATCT3 | 1 = Reporter |
| | | | 2 = Quencher |
| | | | 3 = Phosphate |
| 521 | SC_ESR1EX2WTPRB3B | 1CAGTACCA2ATGACAAGGGAAGTATGGCT3 | 1 = Reporter |
| | | | 2 = Quencher |
| | | | 3 = Phosphate |
| 522 | SC_ESR1EX2WTPRB4 | 1TGACAAGG2GAAGTATGGCTATGGAATCT3 | 1 = Reporter |
| | | | 2 = Quencher |
| | | | 3 = Phosphate |
| 523 | SC_ESR1EX5E380QASR1 | CATGATCAGGTCCACCTTCTAC | |
| 524 | SC_ESR1EX5E380QASR2 | CCATGATCAGGTCCACCTTCTAC | |
| 525 | SC_ESR1EX5E380QASR3 | TCCATGATCAGGTCCACCTTCTAC | |
| 526 | SC_ESR1EX5E380QCRP1 | GAGCAAGTTAGGAGCAAACAGTA | |
| 527 | SC_ESR1EX5E380QCRP2 | AGAGCAAGTTAGGAGCAAACAGTA | |
| 528 | SC_ESR1EX5E380QCRP3 | AAGAGCAAGTTAGGAGCAAACAGTA | |
| 529 | SC_ESR1EX5WTASR1 | CATGATCAGGTCCACCTTCTAG | |
| 530 | SC_ESR1EX5WTCRP1 | GAGCAAGTTAGGAGCAAACAGTA | |
| 531 | SC_ESR1EX5WTCRP2 | AGAGCAAGTTAGGAGCAAACAGTA | |
| 532 | SC_ESR1EX5WTCRP3 | AAGAGCAAGTTAGGAGCAAACAGTA | |
| 533 | SC_ESR1EX5WTPBR1 | 1TGCCTG2GCTAGAGATCCTGATGATTGGT3 | 1 = Reporter |
| | | | 2 = Quencher |
| | | | 3 = Phosphate |
| 534 | SC_ESR1EX6L429DVASR1 | GGTAGAGATCTTCGACATGCTGG | |
| 535 | SC_ESR1EX6L429DVASR2 | ATGGTAGAGATCTTCGACATGCTGG | |
| 536 | SC_ESR1EX6V422DVASR1 | ATGTGTAGAGGGCATGGAGATCT | |
| 537 | SC_ESR1EX6V422DVCRP1 | GTTATCAACTCACCAGAATTAAGCAA | |
| 538 | SC_ESR1EX6V422DVCRP2 | TGTTATCAACTCACCAGAATTAAGCAA | |
| 539 | SC_ESR1EX6V422DVCRP3 | GTGTTATCAACTCACCAGAATTAAGCAA | |
| 540 | SC_ESR1EX6V422DVPRB1 | 1CATCTCGGTT2CCGCATGATGAATCTGC3 | 1 = Reporter |
| | | | 2 = Quencher |
| | | | 3 = Phosphate |
| 541 | SC_ESR1EX6WT2ASR1 | GGTAGAGATCTTCGACATGCTGC | |
| 542 | SC_ESR1EX6WT2CRP1 | GTTATCAACTCACCAGAATTAAGCAA | |
| 543 | SC_ESR1EX6WTASR1 | GGTAGAGATCTTCGACATGCTGC | |
| 544 | SC_ESR1EX6WTCRP1 | TCACCAGAATTAAGCAAAATAATAGATT | |
| 545 | SC_ESR1EX6WTPRB1 | 1CATCTCGGTT2CCGCATGATGAATCTGCA3 | 1 = Reporter |
| | | | 2 = Quencher |
| | | | 3 = Phosphate |
| 546 | SC_ESR1EX7S463PASR1 | ATTCAGGAGTGTACACATTTCTGC | |
| 547 | SC_ESR1EX7WTASR1 | CATTCAGGAGTGTACACATTTCTGT | |
| 548 | SC_ESR1EX7WTCRP1 | ATCAGGTGGATCAAAGTGTCTGT | |
| 549 | SC_ESR1EX7WTPRB1 | 1TCTCTG2GAAGAGAAGGACCATATCCACC3 | 1 = Reporter |
| | | | 2 = Quencher |
| | | | 3 = Phosphate |
| 550 | SC_ESR1EX8D538GASR1 | ACGTGGTGCCCCTCTATGG | |
| 551 | SC_ESR1EX8L536HASR1 | CAAGAACGTGGTGCCCCA | |
| 552 | SC_ESR1EX8L536HASR2 | TGCAAGAACGTGGTGCCCCA | |
| 553 | SC_ESR1EX8L536HASR3 | GTGCAAGAACGTGGTGCCCCA | |
| 554 | SC_ESR1EX8L536HASR4 | GTGCAAGAACGTGGTGCCACA | |
| 555 | SC_ESR1EX8L536PASR1 | CAAGAACGTGGTGCCCCC | |
| 556 | SC_ESR1EX8L536QASR1 | CAAGAACGTGGTGCCCCAG | |
| 557 | SC_ESR1EX8L536RASR1 | CAAGAACGTGGTGCCCCG | |
| 558 | SC_ESR1EX8P535H1ASR1 | TGAAGTGCAAGAACGTGGTGCA | |
| 559 | SC_ESR1EX8P535H1ASR2 | CATGAAGTGCAAGAACGTGGTGCA | |
| 560 | SC_ESR1EX8P535H2ASR1 | TGAAGTACAAGAACGTGGTGCA | |
| 561 | SC_ESR1EX8P535H2ASR2 | ATGAAGTACAAGAACGTGGTGCA | |
| 562 | SC_ESR1EX8P535H2ASR3 | CATGAAGTACAAGAACGTGGTGCA | |
| 563 | SC_ESR1EX8P535H2ASR4 | TGAAGTGCAAGAACGTGATGCA | |
| 564 | SC_ESR1EX8P535H2ASR5 | AGCATGAAGTACAAGAACGTGGTGCA | |
| 565 | SC_ESR1EX8P535H3ASR1 | TGAAGTGCAAGAACGTGGTACA | |
| 566 | SC_ESR1EX8P535H3ASR2 | ATGAAGTGCAAGAACGTGGTACA | |
| 567 | SC_ESR1EX8P535H3ASR3 | CATGAAGTGCAAGAACGTGGTACA | |
| 568 | SC_ESR1EX8V534EASR1 | CATGAAGTGCAAGAACGTGGA | |
| 569 | SC_ESR1EX8V534EASR2 | GCATGAAGTGCAAGAACGTGGA | |
| 570 | SC_ESR1EX8V534EASR3 | AGCATGAAGTGCAAGAACGTGGA | |
| 571 | SC_ESR1EX8WTASR1 | ACGTGGTGCCCCTCTATGAC | |
| 572 | SC_ESR1EX8WTCRP1 | GCTTTGGTCCGTCTCCTCC | |
| 573 | SC_ESR1EX8WTPRB1 | 1CTGCTG2GAGATGCTGGACGCCCACC3 | 1 = Reporter |
| | | | 2 = Quencher |
| | | | 3 = Phosphate |
| 574 | SC_ESR1EX8Y537CASR1 | AGAACGTGGTGCCCCTCTG | |
| 575 | SC_ESR1EX8Y537NASR1 | AGAACGTGGTGCCCCTCA | |
| 576 | SC_ESR1EX8Y537NASR2 | AAGAACGTGGTGCCCCTCA | |
| 577 | SC_ESR1EX8Y537NASR3 | CAAGAACGTGGTGCCCCTCA | |
| 578 | SC_ESR1EX8Y537SASR1 | AGAACGTGGTGCCCCTCTC | |
| 579 | ESR1_S463P_CF02 | CTCCTAGACCTCATCCTCTTTGA | |

### Oligonucleotide Selection for High Sensitivity and Specificity Detection of Mutants

Primers were selected to ensure that mutant signal could be sensitively and specifically detected compared to wild type. A primer pair and probe designed to be specific for the ESR1 K303R mutation and for the L536_D538>P mutation were used detect mutant template added to wild type DNA at a ratio of 1:1600 (100 copies of mutant DNA in 160,000 copies of wild type) and in wild type DNA alone. Allele specific amplification and detection were carried out on the cobas z 480. The data are expressed in Ct curves plotting amplification cycles vs fluorescence signal (indicating detection of the amplification product)

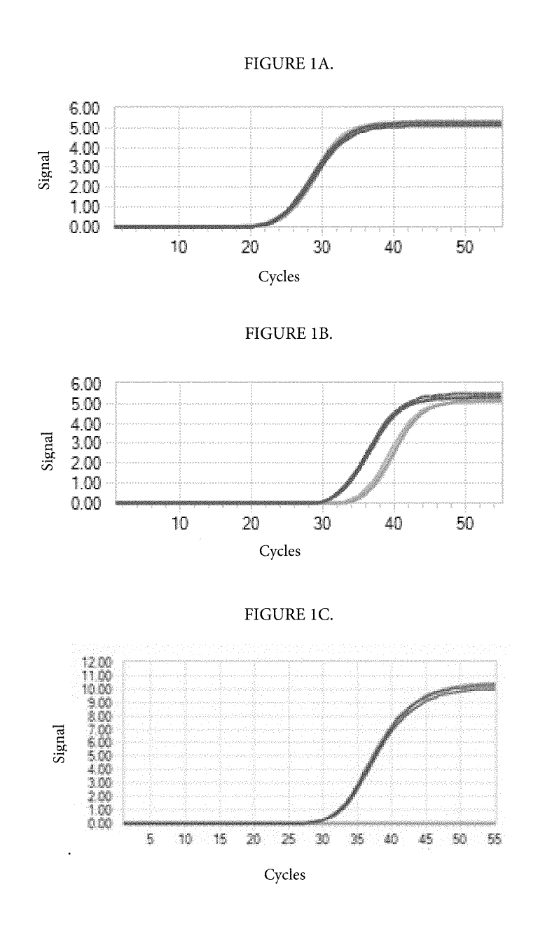

FIG. 1A shows a reaction specific for K303R with no discrimination. The selected primers and probe amplify and detect signal from the mutant and wild type samples at essentially the same level. FIG. 1B shows a reaction specific for K303R with better discrimination. The Ct's of the two wild type samples are shifted to the right by at least 2 cycles. FIG. 1C shows a much more specific reaction for L536_D538>P that is also sensitive enough to detect mutant DNA at a ratio of at least 1:1600. The wild type samples are not detected at all, while the mutant samples have a Ct around 35-38.

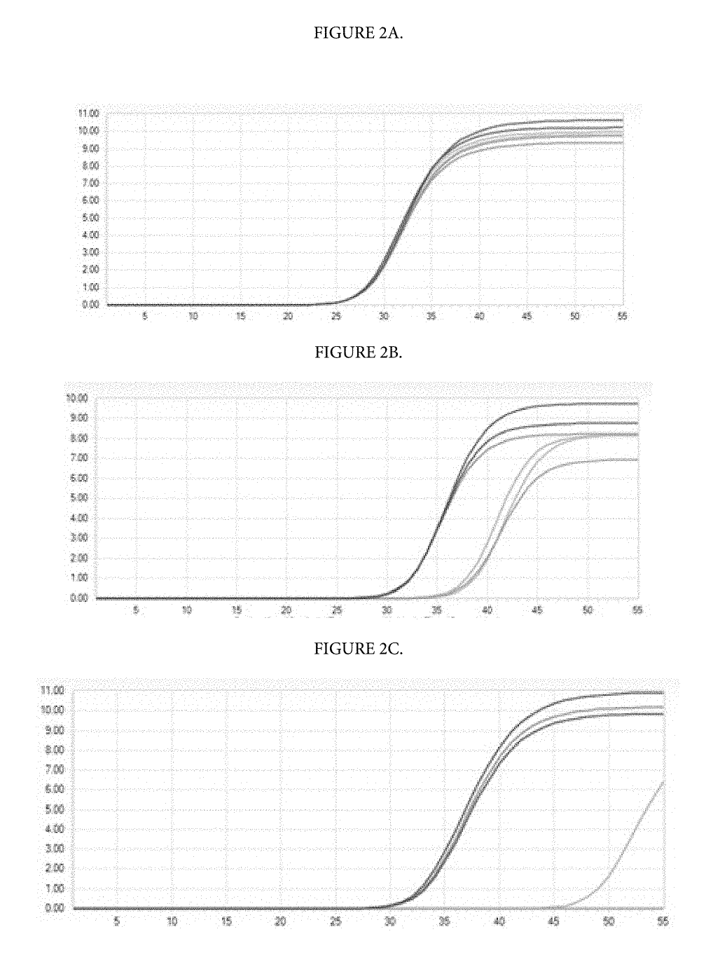

A similar comparison is shown in FIG. 2A, FIG. 2B, and FIG. 2C with primers and probes designed to be specific for the L536R mutation. FIG. 2A shows a reaction with no discrimination using the L536R_RS01 allele-specific primer. Wild type is amplified at essentially the same level as mutant. FIG. 2B shows a reaction with better discrimination using the L536R_RS09 allele-specific primer, where wild type has a Ct delayed by at least 5 cycles compared to mutant. FIG. 2C shows a reaction with good discrimination using the L536R_RS04 allele-specific primer. Wild type has a Ct over 50 cycles, considered not detected, compared to a mutant Ct well in the detectable range around 35.

### Specificity of Multiplex Mutation Detection

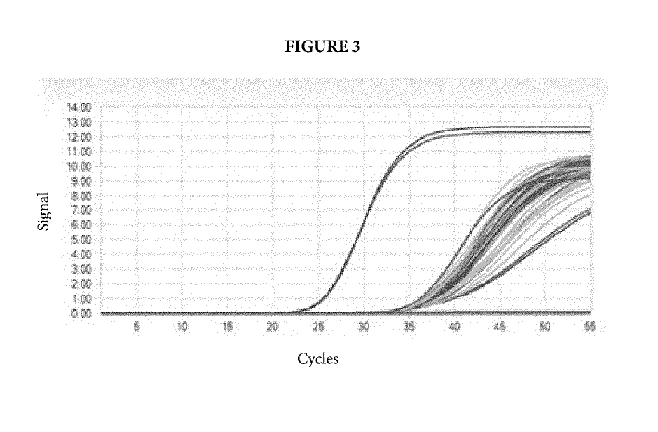

The primer pair and probe sets were tested in multiplex to determine specificity and ensure that they would not detect other ESR1 mutations. Separate sample mixtures were prepared for each of the 18 mutants listed in Table 1, with mutant DNA added 1:1 with wild type (5000 copies of each). Primer pairs and probes specific for all 18 mutations were added. FIG. 3 shows an exemplary Ct curve with high specificity for ESR1 P535H. The two left-most curves with a Ct of around 25-30 represent specific detection of the ESR1 P535H mutation present in the sample (indicated with light arrow). The Ct's for the other primer pair and probe sets have a much more delayed Ct (indicated between two black arrows).

### Assay Linearity

Linearity of detection was also detected across a range of template concentrations for each mutation. As an example primer pair and probe specific for ESR1 S341L were used to amplify 5-5000 copies mutant template in a background of 10,000 wild type copies. The results are shown in Table 3, and show that the assay is highly linear.

TABLE 3

| Mutant copy number | Ct |
|---|---|
| 5 | 33.04 |
| 50 | 29.32 |
| 500 | 26.23 |
| 5000 | 22.89 |
| Slope | −3.36 |
| R² | 0.9987 |

### Detection of ESR1 Mutations in Contrived Plasma Samples

The assay was tested in plasma background to determine if plasma components would interfere. Mutant DNA was added at 1000 copies into 2 ml normal (wild type) plasma. DNA was extracted with the cobas° DNA Sample Preparation Kit. DNA equivalent to that from 0.5 ml plasma was added to each PCR reaction. Primer pairs and probe sets were added corresponding to each mutation. Exemplary results are shown in Table 4. The data show that mutant DNA can be sensitively detected in wild type plasma background.

TABLE 4

| | Average mutant Ct | Average mutant Ct |
|---|---|---|
| Mutation | with mutant added | in plasma only sample |
| L536Q | 28.62 | 45.20 |
| V422delV | 28.55 | ND |
| L536R | 28.55 | 45.20 |
| L536H | 28.40 | 45.20 |
| Y537S | 28.41 | ND |
| E380Q | 28.28 | ND |
| L536P | 29.41 | 45.20 |

### Selected Oligonucleotides

Allele-specific primers that show good specificity for mutant sequence were selected based on the criteria above. These are shown in Table 5 below with the primer name, and SEQ ID NO in parenthesis. Bolded entries indicate primers that show selectivity and specificity in the assay configuration in Table 6 below, though other combinations also perform well.

TABLE 5
Selected allele-specific primers

| Mutation | Selected allele-specific primers |
|---|---|
| K303R | FS02 (479), FS04 (481), FS07 (484) |
| S341L | FS02 (239), FS03 (240), FS05 (242), FS08 (245), FS09 (246), FS10 (247), RS04 |
| | (253), RS05 (254), RS06 (255), RS07 (256), RS08 (257), RS09 (258), RS10 (259) |
| E380Q | FS01 (32), FS03 (34), FS05 (36), FS06 (37), FS07 (38), FS08 (39), FS09 (40), FS10 |
| | (41), FS11 (42) |
| V422DELV | FS01 (287), FS08 (294), FS09 (295), FS10 (296), FS12, FS14 (297), FS15 (298) |
| L429V | FS06 (66), FS08 (68), FS09 (69), FS10 (70), RS01 (72), RS02 (73), RS04 (75), RS05 |
| | (76), RS06 (77), RS07 (78), RS08 (79), RS09 (80), RS10 (81) |
| S463P | FS02 (264), FS03 (265), FS05 (267), FS06 (268), FS07 (269), FS08 (270), FS09 |
| | (271), FS10 (272), RS08 (281), RS09 (282), RS10 (283), RS11 (275), RS12 |
| V533M | FS01 (322), FS08 (329), FS09 (330), RS08 (345), RS09 (346), RS10 (347), RS11 |
| | (348), RS12 (349), RS13 (350), RS15 (352), RS16 (353), RS19 |
| V534E | FS01 (364), FS02 (365), FS04 (367), FS05 (368), FS07 (370), FS08 (371), FS10 (373), |
| | RS01 (378), RS11 (388), RS15 (391), RS16 (392), RS17 (393) |
| P535H | FS01 (181), FS08 (188), FS09 (189), FS10 (190), RS01 (191), RS08 (198), RS09 |
| | (199), RS10 (200), RS13 (203), RS14 (204), RS15 (205), RS18 (208), RS19 (209), |
| | RS22 (212), RS35 (225), RS36 (226), RS38 (228), RS39 (229), RS41 (231) |
| L536H | FS01 (96), FS06 (101), FS09 (104), FS10 (105), RS02 (107), RS05 (110), RS06 (111), |
| | RS07 (112), RS08 (113), RS09 (114), RS10 (115), RS11 (116), RS12 (117) |
| L536P | RS04 (134), RS05 (135), RS07 (137) |
| L536Q | FS01 (141), RS01 (151), RS04 (154), RS05 (155), RS07 (157), RS08 (158), RS09 |
| | (159), RS10 (160) |
| L536R | FS01 (161), RS02 (172), RS04 (174), RS05 (175), RS06 (176), RS07 (177), RS08 |
| | (178), RS09 (179), RS10 (180) |
| Y537C | FS01 (397), RS01 (408), RS08 (415), RS09 (416), RS10 (417), RS11 (418), RS12 |
| | (419), RS13 (420), RS19 (424) |
| Y537N | FS01 (426), RS01 (436), RS06 (441), RS10 (445), RS11 (446), RS12 (447), RS13 |
| | (448), RS14 |
| Y537S | FS01 (449), RS01 (459), RS08 (466), RS09 (467), RS10 (468), RS11 (469), RS12 |
| | (470), RS13 (471), RS14 (472) |
| D538G | RS01 (14), RS08 (21), RS09 (22), RS10 (23), RS11 (24), RS12 (25) |
| L536_D538 > P | RS01 (84), RS02 (85), RS03 (86), RS04 (87), RS05 (88), RS06 (89), RS07 (90), RS08 |
| | (91), RS09 (92), RS10 (93) |

In the case of exon 8 RS primers, any exon 8 CF primer can be used, for example, V533M_CF03 (SEQ ID NO:314) or Y537C_CF01 (SEQ ID NO:394). Any exon 8 probe can be used, for example Y537C_R_P01 (SEQ ID NO:407). In general, for any FS allele-specific primer, any corresponding CR primer and F probe can be used. Similarly for any RS allele-specific primer, any corresponding CF primer and R probe can be used. Particular examples include K303R_CR02 (SEQ ID NO:476) and K303R_F_P01 (SEQ ID NO:477); S341L_CR02 (SEQ ID NO:235) and S341L_F_P02 (SEQ ID NO:237); E380QCRP3 (SEQ ID NO:528) and E380Q_F_P01 (SEQ ID NO:31); V422delV_CR01 (SEQ ID NO:285) and V422delV_F_P01 (SEQ ID NO:286); L429V_CR01 (SEQ ID NO:59) and L429_F_P01 (SEQ ID NO:60); and S463P_CR01 (SEQ ID NO:261) and S463P_F_P01 (SEQ ID NO:262).

### Example Assay Configuration

We sought to design a highly multiplexed assay to detect the maximum number of mutations in a minimal number of reaction vessels. Examples of three-vessel assays are shown below in Tables 6 and 7. Exon 8 mutations are grouped together to allow for use of a common forward primer and probe. If desired, one of skill will understand how to identify the exact exon 8 mutation(s) that results in a mutation positive signal, e.g., using sequencing or PCR with mutation-specific probes.

TABLE 6
Exemplary assay configuration for allele-specific multiplex ESR1 mutation

| Reporter | 1 | 2 | 3 |
|---|---|---|---|
| FAM | L536H/L536P/L536Q/ | Y537C/Y537N/Y537S/ | V533M/ |
| | L536R/D538G | L536_D538 > P | V534E/ |
| | | | P535H |
| HEX | S463P | E380Q | S341L |
| JA270 | V422DELV | K303R | L429V |
| CY5.5 | IC | IC | IC |

TABLE 7
Exemplary assay configuration for allele-specific multiplex ESR1 mutation

| Reporter | 1 | 2 | 3 |
|---|---|---|---|
| FAM | L536H/L536P/L536Q/ | Y537C/Y537N/ | V533M/ |
| | L536R/L536_D538 > P | Y537S/D538G | V534E/ |
| | | | P535H |
| HEX | S463P | S341L | E380Q |
| JA270 | K303R | L429V | V422DELV |
| CY5.5 | IC | IC | IC |

One of skill will understand that different configurations can be made, depending on the mutations of interest and availability of fluorescence channels. For example, the number of tubes can be increased to allow for individual detection of exon 8 mutations. As the number of fluorescence channels increases, the number of mutations that can be included in a single tube will increase.

Detection of E380Q mutation from FFPET Samples in Multiplex Reactions

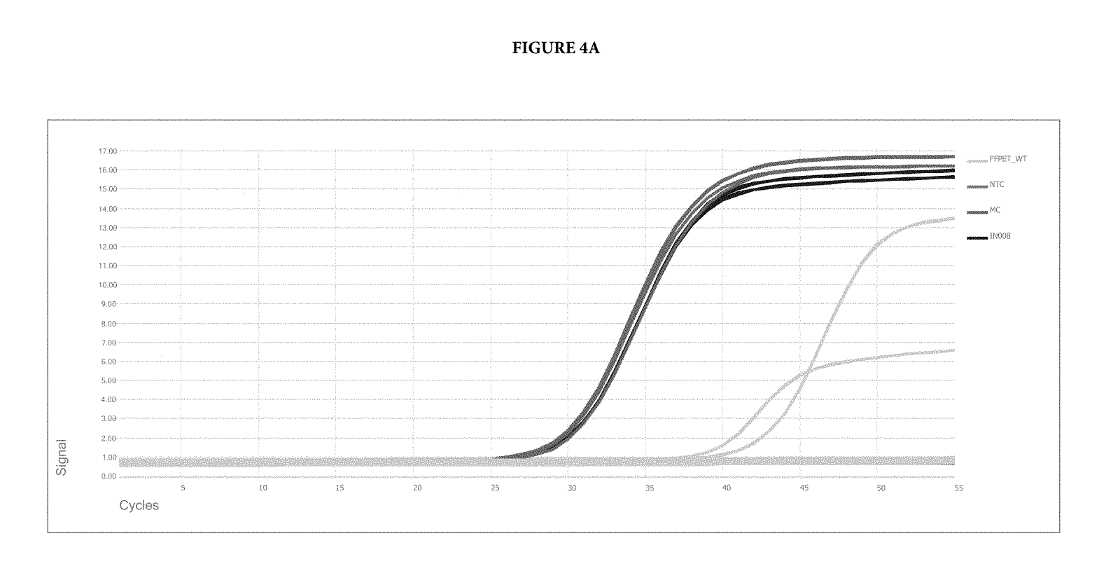

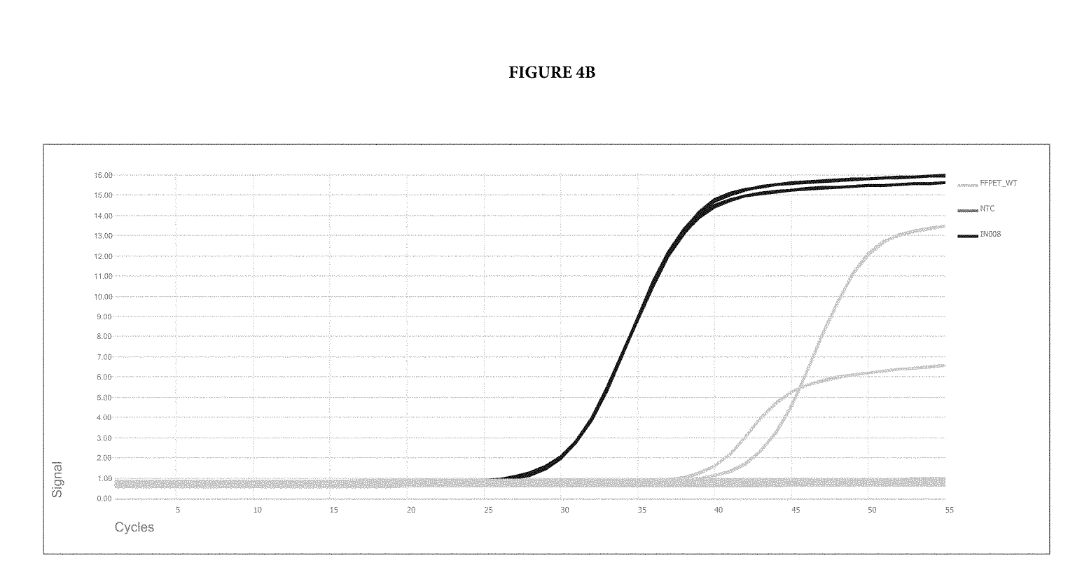

DNA from 142 FFPET samples (breast, lymph node, lung, bone) from cancer patients was extracted using the cobas® DNA Sample Preparation Kit. DNA (50 ng/reaction) was analyzed for ESR1 mutations by the multiplex allele-specific PCR as described herein (performed in duplicate). Results are shown in FIGS. 4A and 4B. One sample (IN008) was found positive for the E380Q mutation, and has a similar amplification growth curve to the E380Q mutation positive control (MC) (see FIG. 4A). FIG. 4B shows the same data without the E380Q mutation positive control. Results were confirmed using ddPCR.

The E380Q mutation positive sample shows a profile that is clearly distinguishable from E380 wild type and non-template control. The results also show that the multiplex assays described herein are effective for detecting specific mutations in DNA from FFPET clinical samples.

Detection of D538G Mutation from Plasma Samples in Multiplex Reactions

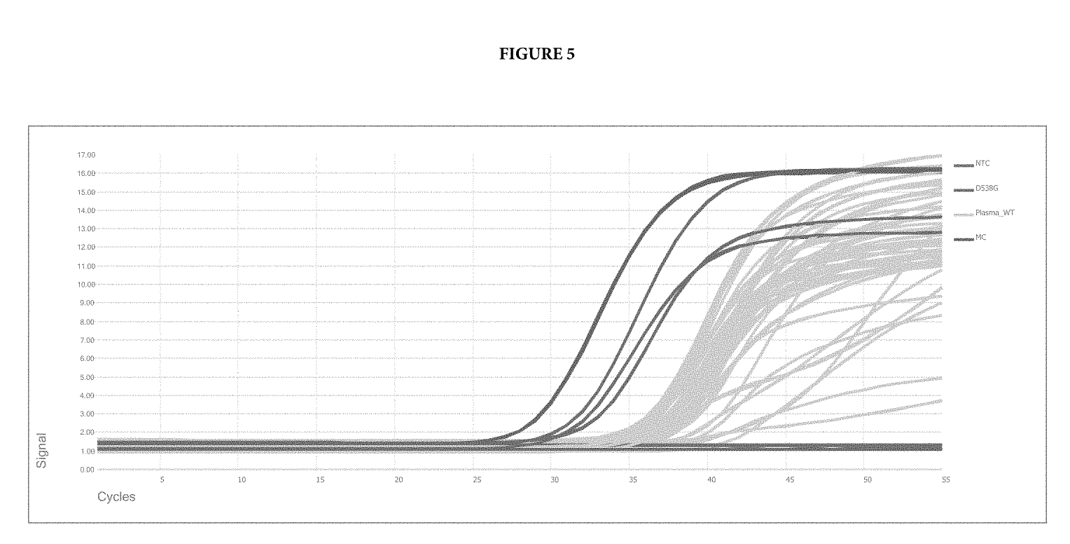

Clinical plasma samples (2 ml plasma) were obtained from 103 post-menopausal metastatic breast cancer patients who relapsed during treatment with an aromatase inhibitor, or within 12 months after discontinuation. DNA was extracted using the cobas® cfDNA Sample Preparation Kit and analyzed for ESR1 mutations by multiplex allele-specific PCR as described herein. Three samples tested positive for D538G, as shown in FIG. 5. FIG. 5 shows results from 51 of the samples, 3 D538G positive and 48 D538 wild type. D538G mutation positive and non-template controls are also shown, and the D538G positive samples show profiles that are dearly distinguishable. Results were confirmed using ddPCR.

The results show that the multiplex assays described herein are effective for detecting specific mutations in DNA from non-invasive (plasma) clinical samples.

While the invention has been described in detail with reference to specific examples, it will be apparent to one skilled in the art that various modifications can be made within the scope of this invention. Thus the scope of the invention should not be limited by the examples described herein. All patents, publications, websites, Genbank (or other database) entries disclosed herein are incorporated by reference in their entireties.

1. A kit comprising one or more vessels, each of the vessels holding two or more primer pairs, each primer pair specific for a different sequence in the ESR1 gene;
two or more probes specific for a different sequence in the ESR1 gene, wherein each probe is labeled with a fluorophore and quencher;
a primer pair specific for an internal control sequence; and
a probe specific for the internal control sequence and labeled with a fluorophore and quencher.

2. The kit of claim 1, comprising three vessels.

3. The kit of claim 2, wherein each of the three vessels holds five to sixteen primer pairs.

4. The kit of claim 2, wherein each of the three vessels holds three to four probes.

5. The kit of claim 1, comprising a primer pair specific for ESR1 V422DelV, a primer pair specific for ESR1 S463P, a primer pair specific for ESR1 L536H, a primer pair specific for ESR1 L536P, a primer pair specific for ESR1 L536Q, a primer pair specific for ESR1 L536R, a primer pair specific for ESR1 D538G, ESR1 K303R, a primer pair specific for ESR1 E380Q, a primer pair specific for ESR1 L536_D538>P, a primer pair specific for ESR1 Y537C, a primer pair specific for ESR1 Y537N, a primer pair specific for ESR1 Y537S, a primer pair specific for ESR1 S341L, a primer pair specific for ESR1 L429V, a primer pair specific for ESR1 V533M, a primer pair specific for ESR1 V534E, and a primer pair specific for ESR1 P535H.

6. The kit of claim 1, comprising:
(i) a first vessel holding a primer pair specific for ESR1 V422DelV, a primer pair specific for ESR1 S463P, a primer pair specific for ESR1 L536H, a primer pair specific for ESR1 L536P, a primer pair specific for ESR1 L536Q, a primer pair specific for L536R, and a primer pair specific for ESR1 D538G;
(ii) a second vessel holding a primer pair specific for ESR1 K303R, a primer pair specific for ESR1 E380Q, a primer pair specific for ESR1 L536_D538>P, a primer pair specific for ESR1 Y537C, a primer pair specific for ESR1 Y537N, and a primer pair specific for ESR1 Y537S; and
(iii) a third vessel holding a primer pair specific for ESR1 S341L, a primer pair specific for ESR1 L429V, a primer pair specific for ESR1 V533M, a primer pair specific for ESR1 V534E, and a primer pair specific for ESR1 P535H.

7. The kit of any one of the foregoing claims, wherein at least one primer in the two or more primer pairs includes a modified, non-naturally occurring nucleotide.

8. (canceled)

9. The kit of claim 1, wherein each vessel further holds a thermostable DNA polymerase.

10. A method for determining the presence or absence of two or more ESR1 mutations in a sample from an individual, comprising:
(i) obtaining a sample from the individual;
(ii) carrying out multiplex allele-specific PCR to determine the presence or absence of two or more ESR1 mutations.

11. The method of claim 10, wherein the individual has breast cancer and is undergoing hormone therapy.

12. The method of claim 10, comprising carrying out multiplex allele-specific PCR to determine the presence or absence of ten or more ESR1 mutations.

13. The method of claim 10, wherein the two or more ESR1 mutations include ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H.

14. The method of claim 10, further comprising providing modified treatment to the individual when the presence of an ESR1 mutation is determined.

15. The method of claim 10, wherein the multiplex allele-specific PCR is carried out in three vessels.

16. The method of claim 15, wherein the presence or absence of ESR1 mutations is determined in the three vessels as follows:
(i) ESR1 V422DelV, ESR1 S463P, ESR1 L536H, ESR1 L536P, ESR1 L536Q, L536R, and ESR1 D538G in a first vessel;
(ii) ESR1 K303R, ESR1 E380Q, ESR1 L536_D538>P, ESR1 Y537C, ESR1 Y537N, and ESR1 Y537S in a second vessel;
(iii) ESR1 S341L, ESR1 L429V, ESR1 V533M, ESR1 V534E, and ESR1 P535H in a third vessel.

17. A method of providing modified treatment of an individual with breast cancer that is on hormone therapy comprising:
(i) obtaining a sample from the individual;
(ii) carrying out multiplex allele-specific PCR to determine the presence or absence of two or more ESR1 mutations;
(iii) providing modified treatment of the individual if the presence of an ESR1 mutation is determined.

18. The method of claim 17, comprising carrying out multiplex allele-specific PCR to determine the presence or absence of ten or more ESR1 mutations.

19. The method of claim 17 or 18, wherein the two or more ESR1 mutations include ESR1 V422DelV, ESR1 S463P, ESR1 L536H, ESR1 L536P, ESR1 L536Q, ESR1 L536R, ESR1 D538G, ESR1 K303R, ESR1 E380Q, ESR1 L536_D538>P, ESR1 Y537C, ESR1 Y537N, ESR1 Y537S, ESR1 S341L, ESR1 L429V, ESR1 V533M, ESR1 V534E, and ESR1 P535H.

20. The method of any one of claims 17-19, wherein steps (i)-(ii) are carried out 0.5-5 years after initiating hormone therapy.

21. The method of any one of claims 17-20 wherein the method is carried out more than once during hormone therapy.

22. The method of any one of claims 17-21, wherein the step (iii) comprises providing an additional hormone therapy or standard chemotherapy to the individual.

23-29. (canceled)
